(19) United States
(12) Patent Application Publication
(10) Pub. No.: US 2025/0381253 A1
(43) Pub. Date: Dec. 18, 2025

(54) MUTANASES AND ORAL CARE COMPOSITIONS COMPRISING SAME

(71) Applicant: Novozymes A/S, Bagsvaerd (DK)

(72) Inventors: Manish Kumar Tiwari, Charlottenlund (DK); Dorotea Raventos SEGURA, Rungsted (DK); Mary Ann STRINGER, Soeborg (DK); Lorena González PALMÉN, Åkarp (SE); Jesper SALOMON, Hotte (DK)

(73) Assignee: Novozymes A/S, Bagsvaerd (DK)

(21) Appl. No.: 18/878,352

(22) PCT Filed: Jun. 28, 2023
(86) PCT No.: PCT/EP2023/067662

§ 371 Date: Dec. 23, 2024

(30) Foreign Application Priority Data
Jun. 30, 2022 (EP) 22182248.9

(51) Int. Cl. A61K 38/47; A61K 8/66; A61P 1/02; A61P 31/04; A61Q 11/00

(52) U.S. Cl. CPC A61K 38/47; A61K 8/66; A61P 1/02; A61P 31/04; A61Q 11/00; C12Y 302/01059

(57) ABSTRACT

The present invention relates to polypeptides having endo-1,3-α-glucanase activity, polynucleotides encoding the polypeptides, nucleic acid constructs, vectors, host cells comprising the polynucleotides, methods of producing the polypeptides, as well as oral care compositions comprising the polypeptides.

### REFERENCE TO A SEQUENCE LISTING

This application contains a Sequence Listing in computer readable form, which is incorporated herein by reference.

### FIELD OF THE INVENTION

The present invention relates to polypeptides having endo-1,3-α-glucanase activity (i.e., mutanases), polynucleotides encoding the polypeptides, nucleic acid constructs, vectors, host cells comprising the polynucleotides, methods of producing the polypeptides, as well as oral care compositions comprising the polypeptides.

### BACKGROUND OF THE INVENTION

Biofilms are communities of bacteria that are found on solid surfaces in many different environments, including surfaces of the oral cavity. Oral biofilm, or dental plague, contains many of the bacteria that are associated with oral health issues such as oral malodor, demineralization, dental caries, tooth decay, potential loss of teeth and gum disease (gingivitis and periodontitis).

The formation of oral biofilm occurs in three stages known as the lag phase, growth phase, and steady state, respectively. In the lag phase, glycoproteins from saliva bind to an oral surface such as teeth and create a structure termed the pellicle that functions as attachment site for bacteria. In the growth phase, co-aggregation occurs, i.e., secondary bacterial colonizers attach to the primary bacterial colonizers, causing the diversity of the biofilm to increase and the biofilm to grow and mature. In the steady state, the biofilm growth slows down and eventually stops. This stage-based formation cycle causes biofilms to exist in several consecutive layers, which makes physical abrasion of biofilm more difficult.

Within a biofilm, the residing bacterial cells are distributed in an extracellular polymeric matrix that consists primarily of water, proteins, exopolysaccharides, lipopolysaccharides, lipids, surfactants, and extracellular DNA, with exopolysaccharides occupying a major fraction of the dry weight of biofilm (H. C. Flemming, and J. Wingender (2010), Nat. Rev. Microbiol. 8, 623-633). The exopolysaccharides are mainly glucose and fructose homopolymers, including 1,3-α-D-glucans, 1,4-α-D-glucans, 1,6-α-D-glucans and 2,6-β-D-fructans. These polysaccharides are synthesized from ingested sucrose by glucosyltransferases and fructosyltransferases secreted by oral bacteria such as Streptococcus spp., Lactobacillus spp., and Actinomyces spp.). Mutans and dextrans are particularly important glucans in the formation of dental plaque. Mutans have a highly branched structure with main chains composed of glucose molecules linked with 1,3-α-glycosidic linkages and 1,6-α-glycosidic linkages in their side chains. Dextrans are also high molecular weight polymers of glucose containing numerous consecutive 1,6-α-linkages in their backbone and side chains, which begin from a 1,3-α-linkage (M. Pleszczynska et al. (2016), Biotechnol. Appl. Biochem. 64(3), 337-346).

Because of the increased resistance to anti-microbial agents as well as the mechanical properties of biofilm, many current oral care products are rather inefficient in addressing biofilm formation and alleviating the associated oral health issues. The main focus for biofilm removal has been on mechanical abrasion. However, this approach is difficult due to the multilayered nature of biofilms and is further compromised by the fact that mechanical removal of biofilm, e.g., by brushing the teeth, expands and deepens the areas in the oral cavity where biofilms attach and expand, thus potentially increasing the severity of the problem rather than reducing it.

In view of the important role of biofilm in oral disease, there is a need in the art for oral care compositions that can effectively target oral biofilm. To this end, enzymatic oral care solutions comprising mutanases (i.e., 1,3-α-glucanases) and/or dextranases are of particular interest. WO 1997/38669 (Novozymes) describes oral care compositions comprising a Trichoderma harzianum mutanase and a Paecilomyces lilacinum dextranase, WO 1998/57653 (Novozymes) provides oral care compositions comprising a Paecilomyces lilacinum dextranase and a Bacillus pullulanase, U.S. Pat. No. 4,353,981 (Guggenheim et al.) describes the use of the Trichoderma harzianum CBS 243.71 mutanase, the Penicillium funiculosum NRRL 1768 mutanase and the Penicillium lilacinum NRRL 896 mutanase for the removal of dental plaque, U.S. Pat. No. 10,472,616 (Genofocus) describes a Trichoderma harzianum GF101 mutanase and its use in degrading bacterial biofilm, and WO 2020/099490 (Novozymes) describes oral care compositions comprising a Trichoderma harzianum mutanase and a microbial DNase. However, there is a still need for further and improved oral care compositions that can more effectively degrade oral biofilm.

An object of the present invention is to provide mutanases with improved thermal stability and oral care compositions comprising said mutanases. A further object of the present invention is to provide mutanases that provide improved biofilm prevention and oral care compositions comprising said mutanases. A further object of the present invention is to provide mutanases that at the same time have improved thermal stability and provide on par or even improved biofilm prevention and oral care compositions comprising said mutanases. A further object of the present invention is to provide mutanases that display enzymatic activity at pH 5-10, preferably at pH 6-9, most preferably at pH 7-8, and oral care compositions comprising said mutanases. A further object of the present invention is to provide mutanases that at the same time have improved thermal stability and display enzymatic activity at pH 5-10, preferably at pH 6-9, most preferably at pH 7-8, and oral care compositions comprising said mutanases. A further object of the present invention is to provide mutanases that at the same time have improved thermal stability, display enzymatic activity at pH 5-10, preferably at pH 6-9, most preferably at pH 7-8, and provide on par or even improved biofilm prevention, and oral care compositions comprising said mutanases.

### SUMMARY OF THE INVENTION

The present invention provides polypeptides having endo-1,3-α-glucanase activity (i.e., mutanases) that have improved thermal stability, provide on par or improved biofilm prevention, and display enzymatic activity at typical intraoral pH levels. The present invention also provides oral care compositions comprising said polypeptides.

In a first aspect, the present invention relates to an oral care composition comprising a mutanase selected from the group consisting of:
(a) a polypeptide having at least 60% sequence identity to SEQ ID NO:3;
(b) a polypeptide having at least 90% sequence identity to SEQ ID NO:6;
(c) a polypeptide having at least 65% sequence identity to SEQ ID NO:9;
(d) a polypeptide having at least 65% sequence identity to SEQ ID NO:12;
(e) a polypeptide having at least 60% sequence identity to SEQ ID NO: 15;
(f) a polypeptide having at least 60% sequence identity to SEQ ID NO: 18;
(g) a polypeptide having at least 60% sequence identity to SEQ ID NO:21;
(h) a polypeptide having at least 60% sequence identity to SEQ ID NO:24;
(i) a polypeptide having at least 60% sequence identity to SEQ ID NO:27;
(j) a polypeptide having at least 60% sequence identity to SEQ ID NO:30; and
(k) a polypeptide having at least 60% sequence identity to SEQ ID NO:33;

wherein the polypeptide has endo-1,3-α-glucanase activity, and wherein the oral care composition further comprises at least one oral care ingredient.

The present invention also relates to use of the oral care compositions of the invention as a medicament, methods of using the oral care compositions of the invention, and kits of parts comprising the oral compositions or the invention.

In a second aspect, the present invention relates to a polypeptide having endo-1,3-α-glucanase activity selected from the group consisting of:
(a) a polypeptide having at least 60% sequence identity to SEQ ID NO:3;
(b) a polypeptide having at least 90% sequence identity to SEQ ID NO:6;
(c) a polypeptide having at least 65% sequence identity to SEQ ID NO:9;
(d) a polypeptide having at least 65% sequence identity to SEQ ID NO: 12;
(e) a polypeptide having at least 60% sequence identity to SEQ ID NO: 15;
(f) a polypeptide having at least 60% sequence identity to SEQ ID NO: 18;
(g) a polypeptide having at least 60% sequence identity to SEQ ID NO:21;
(h) a polypeptide having at least 60% sequence identity to SEQ ID NO:24;
(i) a polypeptide having at least 60% sequence identity to SEQ ID NO:27;
(j) a polypeptide having at least 60% sequence identity to SEQ ID NO:30; and
(k) a polypeptide having at least 60% sequence identity to SEQ ID NO:33.

The present invention also relates to polynucleotides encoding the polypeptides of the present invention; nucleic acid constructs; expression vectors; host cells comprising the polynucleotides; and methods of producing the polypeptides.

### BRIEF DESCRIPTION OF THE FIGURES

FIG. 1 shows an example of the thermal stability data generated using the nanoDSF instrument. Panel A is an example of the data obtained (the ratio of the fluorescence emission at 350 nm to 330 nm) in triplicate for SEQ ID NO:3 as a function of temperature. Panel B shows the first derivative of the raw data in Panel A. The peak maximum in the first derivative plot corresponds to the mid-point of the thermal unfolding transition, referred to as Tm. In this example the Tm corresponds to 69.6° C. at the pH 6 for SEQ ID NO:3 and is highly reproducible within the three replicates.

FIG. 2 shows effects of pH on the enzymatic activity of selected mutanases (SEQ ID NO: 3, SEQ ID NO:9, SEQ ID NO:24, and SEQ ID NO:36). Using standard reaction conditions (50° C. for 10 minutes), endo-1,3-α-glucanase activity was monitored at varying pH values ranging from 4 to 10. Each data point represents the average of three measurements and the error bars represent standard deviations.

### DEFINITIONS

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

Mutanase: The term “mutanase” means a polypeptide having endo-1,3-α-glucanase activity (EC 3.2.1.59) that catalyzes the hydrolytic cleavage of the 1,3-α-glycosidic linkages found in, e.g., mutan. The terms “mutanase” and “1,3-α-glucanase” and the expression “a polypeptide having endo-1,3-α-glucanase activity activity” are used interchangeably throughout this application. For purposes of the present invention, mutanase activity may be determined according to the procedure described in Example 3 below.

Biofilm prevention: The term “biofilm prevention” means the ability of a polypeptide to decrease the amount of a biofilm grown under defined conditions. For purposes of the present invention, biofilm prevention may be determined according to Example 6 herein.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the polypeptide, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” means any step involved in the production of a polypeptide including, but not limited to, transcription, post-transcriptional modification, translation, post-translational modification, and secretion.

Expression vector: An “expression vector” refers to a linear or circular DNA construct comprising a DNA sequence encoding a polypeptide, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a polypeptide, wherein the “extended” polypeptide has endo-1,3-α-glucanase activity.

Fragment: The term “fragment” means a polypeptide having one or more amino acids absent from the amino and/or carboxyl terminus of the mature polypeptide, wherein the fragment has endo-1,3-α-glucanase activity.

Fusion polypeptide: The term “fusion polypeptide” is a polypeptide in which one polypeptide is fused at the N-terminus and/or the C-terminus of a polypeptide of the present invention. A fusion polypeptide is produced by fusing a polynucleotide encoding another polypeptide to a polynucleotide of the present invention, or by fusing two or more polynucleotides of the present invention together. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Introduced: The term “introduced” in the context of inserting a nucleic acid sequence into a cell, means “transfection”, “transformation” or “transduction,” as known in the art.

Isolated: The term “isolated” means a polypeptide, nucleic acid, cell, or other specified material or component that has been separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted polypeptide expressed in a host cell.

Mature polypeptide: The term “mature polypeptide” means a polypeptide in its mature form following N-terminal and/or C-terminal processing (e.g., removal of signal peptide). In one aspect, the mature polypeptide is SEQ ID NO:3. In one aspect, the mature polypeptide is SEQ ID NO: 6. In one aspect, the mature polypeptide is SEQ ID NO:9. In one aspect, the mature polypeptide is SEQ ID NO:12. In one aspect, the mature polypeptide is SEQ ID NO:15. In one aspect, the mature polypeptide is SEQ ID NO: 18. In one aspect, the mature polypeptide is SEQ ID NO: 21. In one aspect, the mature polypeptide is SEQ ID NO:24. In one aspect, the mature polypeptide is SEQ ID NO:27. In one aspect, the mature polypeptide is SEQ ID NO:30. In one aspect, the mature polypeptide is SEQ ID NO:33.

Mature polypeptide coding sequence: The term “mature polypeptide coding sequence” means a polynucleotide that encodes a mature polypeptide having endo-1,3-α-glucanase activity. In one aspect, the mature polypeptide coding sequence is nucleotides 61 to 1389 of SEQ ID NO:1 or a cDNA sequence thereof. In one aspect, the mature polypeptide coding sequence is nucleotides 73 to 2063 of SEQ ID NO:4 or a cDNA sequence thereof. In one aspect, the mature polypeptide coding sequence is nucleotides 61 to 2102 of SEQ ID NO:7 or a cDNA sequence thereof. In one aspect, the mature polypeptide coding sequence is nucleotides 61 to 2111 of SEQ ID NO: 10 or a cDNA sequence thereof. In one aspect, the mature polypeptide coding sequence is nucleotides 124 to 3352 of SEQ ID NO:13. In one aspect, the mature polypeptide coding sequence is nucleotides 76 to 2391 of SEQ ID NO: 16. In one aspect, the mature polypeptide coding sequence is nucleotides 79 to 2226 of SEQ ID NO:19. In one aspect, the mature polypeptide coding sequence is nucleotides 79 to 2310 of SEQ ID NO:22. In one aspect, the mature polypeptide coding sequence is nucleotides 115 to 2184 of SEQ ID NO:25. In one aspect, the mature polypeptide coding sequence is nucleotides 133 to 2028 of SEQ ID NO:28. In one aspect, the mature polypeptide coding sequence is nucleotides 115 to 2436 of SEQ ID NO:31.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a polypeptide. Nucleic acids may be single stranded or double stranded and may include chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature, or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Purified: The term “purified” means a nucleic acid, polypeptide or cell that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

In one aspect, the term “purified” as used herein refers to the polypeptide or cell being essentially free from components (especially insoluble components) from the production organism. In other aspects, the term “purified” refers to the polypeptide being essentially free of insoluble components (especially insoluble components) from the native organism from which it is obtained. In one aspect, the polypeptide is separated from some of the soluble components of the organism and culture medium from which it is recovered. The polypeptide may be purified (i.e., separated) by one or more of the unit operations filtration, precipitation, or chromatography.

Accordingly, the polypeptide may be purified such that only minor amounts of other proteins, in particular, other polypeptides, are present. The term “purified” as used herein may refer to removal of other components, particularly other proteins and most particularly other enzymes present in the cell of origin of the polypeptide. The polypeptide may be “substantially pure”, i.e., free from other components from the organism in which it is produced, e.g., a host organism for recombinantly produced polypeptide. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein. a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure polypeptide is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The polypeptide of the present invention is preferably in a substantially pure form (i.e., the preparation is essentially free of other polypeptide material with which it is natively or recombinantly associated). This can be accomplished, for example by preparing the polypeptide by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, polypeptide or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g. depth filtration (by use of filter aids or packed filter medias, cloth filtration in chamber filters, rotary-drum filtration, drum filtration, rotary vacuum-drum filters, candle filters, horizontal leaf filters or similar, using sheet or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hydro cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

Subsequence: The term “subsequence” means a polynucleotide having one or more nucleotides absent from the 5′ and/or 3′ end of a mature polypeptide coding sequence; wherein the subsequence encodes a fragment having endo-1,3-α-glucanase activity.

Thermal stability: The term “thermal stability” means the stability of polypeptide towards heat and may be defined by the thermal unfolding transition midpoint (Tm) of the polypeptide. For purposes of the present invention, thermal stability may be determined according to Example 4 (thermal stability of a polypeptide alone) or Example 5 (thermal stability of a polypeptide in the presence of an oral care ingredient below as thermal stability defined by the thermal unfolding transition midpoint (Tm). In the context of the present invention, the term “on par thermal stability” means that the thermal stability of a polypeptide is within +/−5% of the thermal stability (Tm value) of another polypeptide. In the context of the present invention, the term “improved thermal stability” means that the thermal stability of a polypeptide is improved more than 5%, e.g., 6%, 7%, 8%, 9%, 10%, 15%, 20%, 25%, 30%, 40%, 50%, 60%, 70%, 80%, 90%, 100%, or even more, compared to the thermal stability of another polypeptide.

Variant: The term “variant” means a polypeptide having endo-1,3-α-glucanase activity comprising a man-made mutation, i.e., a substitution, insertion (including extension), and/or deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### Sequence Overview

SEQ ID NO:1 is the DNA encoding a mutanase obtained from Humicola insolens.
SEQ ID NO:2 is the full Humicola insolens mutanase (including the native signal peptide).
SEQ ID NO:3 is the mature Humicola insolens mutanase.
SEQ ID NO:4 is the DNA sequence encoding a mutanase obtained from Trichoderma reesei.
SEQ ID NO:5 is the full Trichoderma reesei mutanase (including the native signal peptide).
SEQ ID NO:6 is the mature Trichoderma reesei mutanase.
SEQ ID NO:7 is the DNA sequence encoding a mutanase obtained from Talaromyces bacillisporus.
SEQ ID NO:8 is the full Talaromyces bacillisporus mutanase (including the native signal peptide).
SEQ ID NO:9 is the mature Talaromyces bacillisporus mutanase.
SEQ ID NO: 10 is the DNA sequence encoding a mutanase obtained from Talaromyces apiculatus.
SEQ ID NO: 11 is the full Talaromyces apiculatus mutanase (including the native signal peptide).
SEQ ID NO: 12 is the mature Talaromyces apiculatus mutanase.
SEQ ID NO: 13 is the DNA sequence encoding a mutanase obtained from Streptomyces yanii.
SEQ ID NO: 14 is the full Streptomyces yanii mutanase (including the native signal peptide).
SEQ ID NO: 15 is the mature Streptomyces yanii mutanase.
SEQ ID NO: 16 is the DNA sequence encoding a mutanase obtained from Streptomyces alni.
SEQ ID NO: 17 is the full Streptomyces alni mutanase (including the native signal peptide).
SEQ ID NO: 18 is the mature Streptomyces alni mutanase.
SEQ ID NO: 19 is the DNA sequence encoding a mutanase obtained from Oribacterium sinus.
SEQ ID NO:20 is the full Oribacterium sinus mutanase (including the native signal peptide).
SEQ ID NO:21 is the mature Oribacterium sinus mutanase.
SEQ ID NO:22 is the DNA sequence encoding a mutanase obtained from Streptomyces galbus.
SEQ ID NO:23 is the full Streptomyces galbus mutanase (including the native signal peptide).
SEQ ID NO:24 is the mature Streptomyces galbus mutanase.
SEQ ID NO:25 is the DNA sequence encoding a mutanase obtained from Streptomyces glomeratus.
SEQ ID NO:26 is the full Streptomyces glomeratus mutanase (including the native signal peptide).
SEQ ID NO:27 is the mature Streptomyces glomeratus mutanase.
SEQ ID NO:28 is the DNA sequence encoding a mutanase obtained from Tetrasphaera sp-0185.
SEQ ID NO:29 is the full Tetrasphaera sp-0185 mutanase (including the native signal peptide).
SEQ ID NO:30 is the mature Tetrasphaera sp-0185 mutanase.
SEQ ID NO:31 is the DNA sequence encoding a mutanase obtained from Kitasatospora phosalacinea.
SEQ ID NO:32 is the full Kitasatospora phosalacinea mutanase (including the native signal peptide).
SEQ ID NO:33 is the mature Kitasatospora phosalacinea mutanase.
SEQ ID NO:34 is the Bacillus clausii signal peptide (BcSP).

### SEQ ID NO:35 is the HHHHHHPR His-tag.

SEQ ID NO:36 is the mature Trichoderma harzianum mutanase.

### DETAILED DESCRIPTION OF THE INVENTION

### Polypeptides Having Endo-1,3-α-Glucanase Activity

The present invention relates to polypeptides having endo-1,3-α-glucanase activity.

In an aspect, the invention relates to polypeptides having endo-1,3-α-glucanase activity selected from the group consisting of:
(a) a polypeptide having at least 60% sequence identity to SEQ ID NO:2;
(b) a polypeptide having at least 60% sequence identity to SEQ ID NO:3;
(c) a polypeptide having at least 60% sequence identity to a mature polypeptide of SEQ ID NO:2;
(d) a polypeptide encoded by a polynucleotide having at least 60% sequence identity to the mature polypeptide coding sequence of SEQ ID NO:1 or a cDNA sequence thereof;
(e) a polypeptide derived from SEQ ID NO:2, a mature polypeptide of SEQ ID NO:2, or SEQ ID NO:3 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:2 or a mature polypeptide of SEQ ID NO:2.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:3.

The polypeptide preferably comprises, consists essentially of, or consists of the amino acid sequence of SEQ ID NO:2 or a mature polypeptide thereof.

The polypeptide preferably comprises, consists essentially of, or consists of the amino acid sequence of SEQ ID NO:3.

The polypeptide may have an N-terminal and/or C-terminal extension of one or more amino acids, e.g., 1-5 amino acids.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1 or a cDNA sequence thereof.

The polynucleotide encoding the polypeptide preferably comprises, consists essentially of, or consists of nucleotides 1 to 87 and 142 to 1389 of SEQ ID NO: 1, in particular nucleotides 61 to 87 and 142 to 1389 of SEQ ID NO:1.

In another aspect, the polypeptide is derived from SEQ ID NO:2 by substitution, deletion or addition of one or several amino acids. In another aspect, the polypeptide is derived from a mature polypeptide of SEQ ID NO:2 by substitution, deletion or addition of one or several amino acids. In another aspect, the polypeptide is derived from SEQ ID NO:3 by substitution, deletion or addition of one or more amino acids. In some embodiments, the polypeptide is a variant of SEQ ID NO:3 comprising a substitution, deletion, and/or insertion at one or more positions. In one aspect, the number of amino acid substitutions, deletions and/or insertions introduced into the polypeptide of SEQ ID NO:3 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability compared at pH 5 and/or pH 6 to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol

In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

In one embodiment, the polypeptide displays endo-1,3-α-glucanase activity at pH 5-10, preferably at pH 6-9, most preferably at pH 7-8. In a preferred embodiment, the polypeptide has improved endo-1,3-α-glucanase activity compared to SEQ ID NO:36 at pH 5-10, preferably at pH 6-9, most preferably at pH 7-8, when determined according to Example 7 herein. In one embodiment, the polypeptide has improved endo-1,3-α-glucanase activity at pH 5 compared to SEQ ID NO:36 when determined according to Example 7 herein. In one embodiment, the polypeptide has improved endo-1,3-α-glucanase activity at pH 6 compared to SEQ ID NO:36 when determined according to Example 7 herein. In one embodiment, the polypeptide has improved endo-1,3-α-glucanase activity at pH 7 compared to SEQ ID NO:36 when determined according to Example 7 herein. In one embodiment, the polypeptide has improved endo-1,3-α-glucanase activity at pH 8 compared to SEQ ID NO:36 when determined according to Example 7 herein. In one embodiment, the polypeptide has improved endo-1,3-α-glucanase activity at pH 9 compared to SEQ ID NO:36 when determined according to Example 7 herein. In one embodiment, the polypeptide has improved endo-1,3-α-glucanase activity at pH 10 compared to SEQ ID NO:36 when determined according to Example 7 herein.

The polypeptide may be a fusion polypeptide.

In an aspect, the polypeptide is isolated.

In another aspect, the polypeptide is purified.

In an aspect, the invention relates to polypeptides having endo-1,3-α-glucanase activity selected from the group consisting of:
(a) a polypeptide having at least 90% sequence identity to SEQ ID NO:5;
(b) a polypeptide having at least 90% sequence identity to SEQ ID NO:6;
(c) a polypeptide having at least 90% sequence identity to a mature polypeptide of SEQ ID NO:5;
(d) a polypeptide encoded by a polynucleotide having at least 90% sequence identity to the mature polypeptide coding sequence of SEQ ID NO:4 or a cDNA sequence thereof;
(e) a polypeptide derived from SEQ ID NO:5, a mature polypeptide of SEQ ID NO:5, or SEQ ID NO:6 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 90%, e.g., at least 91%, at least 92%, at least 93%, at least 94%, at least 95%, at least 96%, at least 97%, at least 98%, at least 99%, or 100% to SEQ ID NO:5 or a mature polypeptide of SEQ ID NO:5.

In another aspect, the polypeptide has a sequence identity of at least 90%, e.g., at least 91%, at least 92%, at least 93%, at least 94%, at least 95%, at least 96%, at least 97%, at least 98%, at least 99%, or 100% to SEQ ID NO:6.

The polypeptide preferably comprises, consists essentially of, or consists of the amino acid sequence of SEQ ID NO:5 or a mature polypeptide thereof.

The polypeptide preferably comprises, consists essentially of, or consists of the amino acid sequence of SEQ ID NO:6.

The polypeptide may have an N-terminal and/or C-terminal extension of one or more amino acids, e.g., 1-5 amino acids.

In some embodiments, the polypeptide is encoded by a polynucleotide having a sequence identity of at least 90%, e.g., at least 91%, at least 92%, at least 93%, at least 94%, at least 95%, at least 96%, at least 97%, at least 98%, at least 99%, or 100% to the mature polypeptide coding sequence of SEQ ID NO:4 or a cDNA sequence thereof.

The polynucleotide encoding the polypeptide preferably comprises, consists essentially of, or consists of nucleotides 1 to 150, 219 to 635, 705 to 1825, and 1883 to 2036 of SEQ ID NO:4, in particular nucleotides 73 to 150, 219 to 635, 705 to 1825, and 1883 to 2036 of SEQ ID NO:4.

In another aspect, the polypeptide is derived from SEQ ID NO:5 by substitution, deletion or addition of one or several amino acids. In another aspect, the polypeptide is derived from a mature polypeptide of SEQ ID NO:5 by substitution, deletion or addition of one or several amino acids. In another aspect, the polypeptide is derived from SEQ ID NO:6 by substitution, deletion or addition of one or more amino acids. In some embodiments, the polypeptide is a variant of SEQ ID NO:6 comprising a substitution, deletion, and/or insertion at one or more positions. In one aspect, the number of amino acid substitutions, deletions and/or insertions introduced into the polypeptide of SEQ ID NO:6 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the polypeptide is a variant of SEQ ID NO:6 having endo-1,3-α-glucanase activity and comprising the amino acid residues Thr at position 31 and Met at position 323, wherein the variant has a sequence identity of at least 90%, e.g., at least 91%, at least 92%, at least 93%, at least 94%, at least 95%, at least 96%, at least 97%, at least 98%, at least 99%, but not 100%, to SEQ ID NO:6, and wherein position numbers are based on the numbering of SEQ ID NO: 6. In one embodiment, the polypeptide comprises, consists essentially of, of consists of SEQ ID NO:6 with Thr at position 31 and Met at position 323.

For purpose of the present invention the following nomenclature is used to describe amino acid substitutions: original amino acid, position, substituted amino acid. Accordingly, the substitution of threonine at position 323 with methionine is designated as “Thr323Met” or “T323M”.

Thus, in another aspect, the polypeptide is a variant of SEQ ID NO:6 having endo-1,3-α-glucanase activity and comprising the substitutions X31T (e.g., I31T) and X323M (e.g., T323M), wherein the variant has a sequence identity of at least 90%, e.g., at least 91%, at least 92%, at least 93%, at least 94%, at least 95%, at least 96%, at least 97%, at least 98%, at least 99%, but not 100%, to SEQ ID NO:6, and wherein position numbers are based on the numbering of SEQ ID NO: 6. In one embodiment, the polypeptide comprises, consists essentially of, of consists of SEQ ID NO:6 with the substitutions I31T and T323M.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability at pH 5 and/or pH 6 compared to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol

In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

In one embodiment, the polypeptide displays endo-1,3-α-glucanase activity at pH 5-10, preferably at pH 6-9, most preferably at pH 7-8. In a preferred embodiment, the polypeptide has improved endo-1,3-α-glucanase activity compared to SEQ ID NO:36 at pH 5-10, preferably at pH 6-9, most preferably at pH 7-8, when determined according to Example 7 herein. In one embodiment, the polypeptide has improved endo-1,3-α-glucanase activity at pH 5 compared to SEQ ID NO:36 when determined according to Example 7 herein. In one embodiment, the polypeptide has improved endo-1,3-α-glucanase activity at pH 6 compared to SEQ ID NO:36 when determined according to Example 7 herein. In one embodiment, the polypeptide has improved endo-1,3-α-glucanase activity at pH 7 compared to SEQ ID NO:36 when determined according to Example 7 herein. In one embodiment, the polypeptide has improved endo-1,3-α-glucanase activity at pH 8 compared to SEQ ID NO:36 when determined according to Example 7 herein. In one embodiment, the polypeptide has improved endo-1,3-α-glucanase activity at pH 9 compared to SEQ ID NO:36 when determined according to Example 7 herein. In one embodiment, the polypeptide has improved endo-1,3-α-glucanase activity at pH 10 compared to SEQ ID NO:36 when determined according to Example 7 herein.

The polypeptide may be a fusion polypeptide.

In an aspect, the polypeptide is isolated.

In another aspect, the polypeptide is purified.

In an aspect, the invention relates to polypeptides having endo-1,3-α-glucanase activity selected from the group consisting of:
(a) a polypeptide having at least 60% sequence identity to SEQ ID NO:8;
(b) a polypeptide having at least 60% sequence identity to SEQ ID NO:9;
(c) a polypeptide having at least 60% sequence identity to a mature polypeptide of SEQ ID NO:8;
(d) a polypeptide encoded by a polynucleotide having at least 60% sequence identity to the mature polypeptide coding sequence of SEQ ID NO:7 or a cDNA sequence thereof;
(e) a polypeptide derived from SEQ ID NO:8, a mature polypeptide of SEQ ID NO:8, or SEQ ID NO:9 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 65%, e.g., at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:8 or a mature polypeptide of SEQ ID NO:8.

In another aspect, the polypeptide has a sequence identity of at least 65%, e.g., at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:9.

The polypeptide preferably comprises, consists essentially of, or consists of the amino acid sequence of SEQ ID NO:8 or a mature polypeptide thereof.

The polypeptide preferably comprises, consists essentially of, or consists of the amino acid sequence of SEQ ID NO:9.

The polypeptide may have an N-terminal and/or C-terminal extension of one or more amino acids, e.g., 1-5 amino acids.

In some embodiments, the polypeptide is encoded by a polynucleotide having a sequence identity of at least 65%, e.g., at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO:7 or a cDNA sequence thereof.

The polynucleotide encoding the polypeptide preferably comprises, consists essentially of, or consists of nucleotides 1 to 129, 196 to 727, 782 to 1007, 1063 to 1520, and 1582 to 2102 of SEQ ID NO:7, in particular nucleotides 61 to 129, 196 to 727, 782 to 1007, 1063 to 1520, and 1582 to 2102 of SEQ ID NO:7.

In another aspect, the polypeptide is derived from SEQ ID NO:8 by substitution, deletion or addition of one or several amino acids. In another aspect, the polypeptide is derived from a mature polypeptide of SEQ ID NO:8 by substitution, deletion or addition of one or several amino acids. In another aspect, the polypeptide is derived from SEQ ID NO:9 by substitution, deletion or addition of one or more amino acids. In some embodiments, the polypeptide is a variant of SEQ ID NO:9 comprising a substitution, deletion, and/or insertion at one or more positions. In one aspect, the number of amino acid substitutions, deletions and/or insertions introduced into the polypeptide of SEQ ID NO:9 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an aminoterminal methionine residue; a small linker peptide of up to 20-25 residues; or a small extension that facilitates purification by changing net charge or another function, such as a poly-histidine tract, an antigenic epitope or a binding module.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability at pH 5 and/or pH 6 compared to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol

In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

In one embodiment, the polypeptide displays endo-1,3-α-glucanase activity at pH 5-10, preferably at pH 6-9, most preferably at pH 7-8. In a preferred embodiment, the polypeptide has improved endo-1,3-α-glucanase activity compared to SEQ ID NO:36 at pH 5-10, preferably at pH 6-9, most preferably at pH 7-8, when determined according to Example 7 herein. In one embodiment, the polypeptide has improved endo-1,3-α-glucanase activity at pH 5 compared to SEQ ID NO:36 when determined according to Example 7 herein. In one embodiment, the polypeptide has improved endo-1,3-α-glucanase activity at pH 6 compared to SEQ ID NO:36 when determined according to Example 7 herein. In one embodiment, the polypeptide has improved endo-1,3-α-glucanase activity at pH 7 compared to SEQ ID NO:36 when determined according to Example 7 herein. In one embodiment, the polypeptide has improved endo-1,3-α-glucanase activity at pH 8 compared to SEQ ID NO:36 when determined according to Example 7 herein. In one embodiment, the polypeptide has improved endo-1,3-α-glucanase activity at pH 9 compared to SEQ ID NO:36 when determined according to Example 7 herein. In one embodiment, the polypeptide has improved endo-1,3-α-glucanase activity at pH 10 compared to SEQ ID NO:36 when determined according to Example 7 herein.

The polypeptide may be a fusion polypeptide.

In an aspect, the polypeptide is isolated.

In another aspect, the polypeptide is purified.

In an aspect, the invention relates to polypeptides having endo-1,3-α-glucanase activity selected from the group consisting of:
(a) a polypeptide having at least 60% sequence identity to SEQ ID NO:11;
(b) a polypeptide having at least 60% sequence identity to SEQ ID NO: 12;
(c) a polypeptide having at least 60% sequence identity to a mature polypeptide of SEQ ID NO:11;
(d) a polypeptide encoded by a polynucleotide having at least 60% sequence identity to the mature polypeptide coding sequence of SEQ ID NO: 10 or a cDNA sequence thereof;
(e) a polypeptide derived from SEQ ID NO: 11, a mature polypeptide of SEQ ID NO: 11, or SEQ ID NO: 12 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 65%, e.g., at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 11 or a mature polypeptide of SEQ ID NO:11.

In another aspect, the polypeptide has a sequence identity of at least 65%, e.g., at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 12.

The polypeptide preferably comprises, consists essentially of, or consists of the amino acid sequence of SEQ ID NO:11 or a mature polypeptide thereof.

The polypeptide preferably comprises, consists essentially of, or consists of the amino acid sequence of SEQ ID NO:12.

The polypeptide may have an N-terminal and/or C-terminal extension of one or more amino acids, e.g., 1-5 amino acids.

In some embodiments, the polypeptide is encoded by a polynucleotide having a sequence identity of at least 65%, e.g., at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 10 or a cDNA sequence thereof.

The polynucleotide encoding the polypeptide preferably comprises, consists essentially of, or consists of nucleotides 1 to 129, 190 to 721, 770 to 995, 1051 to 1511, and 1573 to 2111 of SEQ ID NO: 10, in particular nucleotides 61 to 129, 196 to 727, 782 to 1007, 1063 to 1520, and 1582 to 2102 of SEQ ID NO:10

In another aspect, the polypeptide is derived from SEQ ID NO:11 by substitution, deletion or addition of one or several amino acids. In another aspect, the polypeptide is derived from a mature polypeptide of SEQ ID NO:11 by substitution, deletion or addition of one or several amino acids. In another aspect, the polypeptide is derived from SEQ ID NO: 12 by substitution, deletion or addition of one or more amino acids. In some embodiments, the polypeptide is a variant of SEQ ID NO:12 comprising a substitution, deletion, and/or insertion at one or more positions. In one aspect, the number of amino acid substitutions, deletions and/or insertions introduced into the polypeptide of SEQ ID NO:9 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability at pH 5 and/or pH 6 compared to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol

In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

In one embodiment, the polypeptide displays endo-1,3-α-glucanase activity at pH 5-10, preferably at pH 6-9, most preferably at pH 7-8. In a preferred embodiment, the polypeptide has improved endo-1,3-α-glucanase activity compared to SEQ ID NO:36 at pH 5-10, preferably at pH 6-9, most preferably at pH 7-8, when determined according to Example 7 herein. In one embodiment, the polypeptide has improved endo-1,3-α-glucanase activity at pH 5 compared to SEQ ID NO:36 when determined according to Example 7 herein. In one embodiment, the polypeptide has improved endo-1,3-α-glucanase activity at pH 6 compared to SEQ ID NO:36 when determined according to Example 7 herein. In one embodiment, the polypeptide has improved endo-1,3-α-glucanase activity at pH 7 compared to SEQ ID NO:36 when determined according to Example 7 herein. In one embodiment, the polypeptide has improved endo-1,3-α-glucanase activity at pH 8 compared to SEQ ID NO:36 when determined according to Example 7 herein. In one embodiment, the polypeptide has improved endo-1,3-α-glucanase activity at pH 9 compared to SEQ ID NO:36 when determined according to Example 7 herein. In one embodiment, the polypeptide has improved endo-1,3-α-glucanase activity at pH 10 compared to SEQ ID NO:36 when determined according to Example 7 herein.

The polypeptide may be a fusion polypeptide.

In an aspect, the polypeptide is isolated.

In another aspect, the polypeptide is purified.

In an aspect, the invention relates to polypeptides having endo-1,3-α-glucanase activity selected from the group consisting of:
(a) a polypeptide having at least 60% sequence identity to SEQ ID NO: 14;
(b) a polypeptide having at least 60% sequence identity to SEQ ID NO: 15;
(c) a polypeptide having at least 60% sequence identity to a mature polypeptide of SEQ ID NO:14;
(d) a polypeptide encoded by a polynucleotide having at least 60% sequence identity to the mature polypeptide coding sequence of SEQ ID NO: 13;
(e) a polypeptide derived from SEQ ID NO:14, a mature polypeptide of SEQ ID NO: 14, or SEQ ID NO: 15 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 14 or a mature polypeptide of SEQ ID NO: 14.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:15.

The polypeptide preferably comprises, consists essentially of, or consists of the amino acid sequence of SEQ ID NO: 14 or a mature polypeptide thereof.

The polypeptide preferably comprises, consists essentially of, or consists of the amino acid sequence of SEQ ID NO: 15.

The polypeptide may have an N-terminal and/or C-terminal extension of one or more amino acids, e.g., 1-5 amino acids.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO:13.

The polynucleotide encoding the polypeptide preferably comprises, consists essentially of, or consists of nucleotides 1 to 3352 of SEQ ID NO: 13, in particular nucleotides 124 to 3352 of SEQ ID NO:13.

In another aspect, the polypeptide is derived from SEQ ID NO: 14 by substitution, deletion or addition of one or several amino acids. In another aspect, the polypeptide is derived from a mature polypeptide of SEQ ID NO: 14 by substitution, deletion or addition of one or several amino acids. In another aspect, the polypeptide is derived from SEQ ID NO:3 by substitution, deletion or addition of one or more amino acids. In some embodiments, the polypeptide is a variant of SEQ ID NO:15 comprising a substitution, deletion, and/or insertion at one or more positions. In one aspect, the number of amino acid substitutions, deletions and/or insertions introduced into the polypeptide of SEQ ID NO: 15 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding module.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability at pH 5 and/or pH 6 compared to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol

In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

In one embodiment, the polypeptide displays endo-1,3-α-glucanase activity at pH 5-10, preferably at pH 6-9, most preferably at pH 7-8. In a preferred embodiment, the polypeptide has improved endo-1,3-α-glucanase activity compared to SEQ ID NO:36 at pH 5-10, preferably at pH 6-9, most preferably at pH 7-8, when determined according to Example 7 herein. In one embodiment, the polypeptide has improved endo-1,3-α-glucanase activity at pH 5 compared to SEQ ID NO:36 when determined according to Example 7 herein. In one embodiment, the polypeptide has improved endo-1,3-α-glucanase activity at pH 6 compared to SEQ ID NO:36 when determined according to Example 7 herein. In one embodiment, the polypeptide has improved endo-1,3-α-glucanase activity at pH 7 compared to SEQ ID NO:36 when determined according to Example 7 herein. In one embodiment, the polypeptide has improved endo-1,3-α-glucanase activity at pH 8 compared to SEQ ID NO:36 when determined according to Example 7 herein. In one embodiment, the polypeptide has improved endo-1,3-α-glucanase activity at pH 9 compared to SEQ ID NO:36 when determined according to Example 7 herein. In one embodiment, the polypeptide has improved endo-1,3-α-glucanase activity at pH 10 compared to SEQ ID NO:36 when determined according to Example 7 herein.

The polypeptide may be a fusion polypeptide.

In an aspect, the polypeptide is isolated.

In another aspect, the polypeptide is purified.

In an aspect, the invention relates to polypeptides having endo-1,3-α-glucanase activity selected from the group consisting of:
(a) a polypeptide having at least 60% sequence identity to SEQ ID NO: 17;
(b) a polypeptide having at least 60% sequence identity to SEQ ID NO: 18;
(c) a polypeptide having at least 60% sequence identity to a mature polypeptide of SEQ ID NO:17;
(d) a polypeptide encoded by a polynucleotide having at least 60% sequence identity to the mature polypeptide coding sequence of SEQ ID NO: 16;
(e) a polypeptide derived from SEQ ID NO: 17, a mature polypeptide of SEQ ID NO: 17, or SEQ ID NO: 18 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 17 or a mature polypeptide of SEQ ID NO: 17.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 18.

The polypeptide preferably comprises, consists essentially of, or consists of the amino acid sequence of SEQ ID NO: 17 or a mature polypeptide thereof.

The polypeptide preferably comprises, consists essentially of, or consists of the amino acid sequence of SEQ ID NO: 18.

The polypeptide may have an N-terminal and/or C-terminal extension of one or more amino acids, e.g., 1-5 amino acids.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 16.

The polynucleotide encoding the polypeptide preferably comprises, consists essentially of, or consists of nucleotides 1 to 2391 of SEQ ID NO: 16, in particular nucleotides 76 to 2391 of SEQ ID NO: 16.

In another aspect, the polypeptide is derived from SEQ ID NO:17 by substitution, deletion or addition of one or several amino acids. In another aspect, the polypeptide is derived from a mature polypeptide of SEQ ID NO: 17 by substitution, deletion or addition of one or several amino acids. In another aspect, the polypeptide is derived from SEQ ID NO: 18 by substitution, deletion or addition of one or more amino acids. In some embodiments, the polypeptide is a variant of SEQ ID NO: 18 comprising a substitution, deletion, and/or insertion at one or more positions. In one aspect, the number of amino acid substitutions, deletions and/or insertions introduced into the polypeptide of SEQ ID NO: 18 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding module.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability at pH 5 and/or pH 6 compared to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol

In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

In one embodiment, the polypeptide displays endo-1,3-α-glucanase activity at pH 5-10, preferably at pH 6-9, most preferably at pH 7-8. In a preferred embodiment, the polypeptide has improved endo-1,3-α-glucanase activity compared to SEQ ID NO:36 at pH 5-10, preferably at pH 6-9, most preferably at pH 7-8, when determined according to Example 7 herein. In one embodiment, the polypeptide has improved endo-1,3-α-glucanase activity at pH 5 compared to SEQ ID NO:36 when determined according to Example 7 herein. In one embodiment, the polypeptide has improved endo-1,3-α-glucanase activity at pH 6 compared to SEQ ID NO:36 when determined according to Example 7 herein. In one embodiment, the polypeptide has improved endo-1,3-α-glucanase activity at pH 7 compared to SEQ ID NO:36 when determined according to Example 7 herein. In one embodiment, the polypeptide has improved endo-1,3-α-glucanase activity at pH 8 compared to SEQ ID NO:36 when determined according to Example 7 herein. In one embodiment, the polypeptide has improved endo-1,3-α-glucanase activity at pH 9 compared to SEQ ID NO:36 when determined according to Example 7 herein. In one embodiment, the polypeptide has improved endo-1,3-α-glucanase activity at pH 10 compared to SEQ ID NO:36 when determined according to Example 7 herein.

The polypeptide may be a fusion polypeptide.

In an aspect, the polypeptide is isolated.

In another aspect, the polypeptide is purified.

In an aspect, the invention relates to polypeptides having endo-1,3-α-glucanase activity selected from the group consisting of:
(a) a polypeptide having at least 60% sequence identity to SEQ ID NO:20;
(b) a polypeptide having at least 60% sequence identity to SEQ ID NO:21;
(c) a polypeptide having at least 60% sequence identity to a mature polypeptide of SEQ ID NO:20;
(d) a polypeptide encoded by a polynucleotide having at least 60% sequence identity to the mature polypeptide coding sequence of SEQ ID NO:19;
(e) a polypeptide derived from SEQ ID NO:20, a mature polypeptide of SEQ ID NO:20, or SEQ ID NO:21 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:20 or a mature polypeptide of SEQ ID NO:20.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:21.

The polypeptide preferably comprises, consists essentially of, or consists of the amino acid sequence of SEQ ID NO:20 or a mature polypeptide thereof.

The polypeptide preferably comprises, consists essentially of, or consists of the amino acid sequence of SEQ ID NO:21.

The polypeptide may have an N-terminal and/or C-terminal extension of one or more amino acids, e.g., 1-5 amino acids.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 19.

The polynucleotide encoding the polypeptide preferably comprises, consists essentially of, or consists of nucleotides 1 to 2226 of SEQ ID NO: 19, in particular nucleotides 79 to 2226 of SEQ ID NO: 19.

In another aspect, the polypeptide is derived from SEQ ID NO:20 by substitution, deletion or addition of one or several amino acids. In another aspect, the polypeptide is derived from a mature polypeptide of SEQ ID NO:20 by substitution, deletion or addition of one or several amino acids. In another aspect, the polypeptide is derived from SEQ ID NO:21 by substitution, deletion or addition of one or more amino acids. In some embodiments, the polypeptide is a variant of SEQ ID NO:21 comprising a substitution, deletion, and/or insertion at one or more positions. In one aspect, the number of amino acid substitutions, deletions and/or insertions introduced into the polypeptide of SEQ ID NO:21 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding module.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability at pH 5 and/or pH 6 compared to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol

In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

In one embodiment, the polypeptide displays endo-1,3-α-glucanase activity at pH 5-10, preferably at pH 6-9, most preferably at pH 7-8. In a preferred embodiment, the polypeptide has improved endo-1,3-α-glucanase activity compared to SEQ ID NO:36 at pH 5-10, preferably at pH 6-9, most preferably at pH 7-8, when determined according to Example 7 herein. In one embodiment, the polypeptide has improved endo-1,3-α-glucanase activity at pH 5 compared to SEQ ID NO:36 when determined according to Example 7 herein. In one embodiment, the polypeptide has improved endo-1,3-α-glucanase activity at pH 6 compared to SEQ ID NO:36 when determined according to Example 7 herein. In one embodiment, the polypeptide has improved endo-1,3-α-glucanase activity at pH 7 compared to SEQ ID NO:36 when determined according to Example 7 herein. In one embodiment, the polypeptide has improved endo-1,3-α-glucanase activity at pH 8 compared to SEQ ID NO:36 when determined according to Example 7 herein. In one embodiment, the polypeptide has improved endo-1,3-α-glucanase activity at pH 9 compared to SEQ ID NO:36 when determined according to Example 7 herein. In one embodiment, the polypeptide has improved endo-1,3-α-glucanase activity at pH 10 compared to SEQ ID NO:36 when determined according to Example 7 herein.

The polypeptide may be a fusion polypeptide.

In an aspect, the polypeptide is isolated.

In another aspect, the polypeptide is purified.

In an aspect, the invention relates to polypeptides having endo-1,3-α-glucanase activity selected from the group consisting of:
(a) a polypeptide having at least 60% sequence identity to SEQ ID NO:23;
(b) a polypeptide having at least 60% sequence identity to SEQ ID NO:24;
(c) a polypeptide having at least 60% sequence identity to a mature polypeptide of SEQ ID NO:23;
(d) a polypeptide encoded by a polynucleotide having at least 60% sequence identity to the mature polypeptide coding sequence of SEQ ID NO:22;
(e) a polypeptide derived from SEQ ID NO:23, a mature polypeptide of SEQ ID NO:23, or SEQ ID NO:24 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:23 or a mature polypeptide of SEQ ID NO:23.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:24.

The polypeptide preferably comprises, consists essentially of, or consists of the amino acid sequence of SEQ ID NO:23 or a mature polypeptide thereof.

The polypeptide preferably comprises, consists essentially of, or consists of the amino acid sequence of SEQ ID NO:24.

The polypeptide may have an N-terminal and/or C-terminal extension of one or more amino acids, e.g., 1-5 amino acids.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO:22.

The polynucleotide encoding the polypeptide preferably comprises, consists essentially of, or consists of nucleotides 1 to 2310 of SEQ ID NO:22, in particular nucleotides 79 to 2310 of SEQ ID NO: 22.

In another aspect, the polypeptide is derived from SEQ ID NO:23 by substitution, deletion or addition of one or several amino acids. In another aspect, the polypeptide is derived from a mature polypeptide of SEQ ID NO:23 by substitution, deletion or addition of one or several amino acids. In another aspect, the polypeptide is derived from SEQ ID NO:24 by substitution, deletion or addition of one or more amino acids. In some embodiments, the polypeptide is a variant of SEQ ID NO:24 comprising a substitution, deletion, and/or insertion at one or more positions. In one aspect, the number of amino acid substitutions, deletions and/or insertions introduced into the polypeptide of SEQ ID NO:24 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding module.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability at pH 5 and/or pH 6 compared to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol

In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

In one embodiment, the polypeptide displays endo-1,3-α-glucanase activity at pH 5-10, preferably at pH 6-9, most preferably at pH 7-8. In a preferred embodiment, the polypeptide has improved endo-1,3-α-glucanase activity compared to SEQ ID NO:36 at pH 5-10, preferably at pH 6-9, most preferably at pH 7-8, when determined according to Example 7 herein. In one embodiment, the polypeptide has improved endo-1,3-α-glucanase activity at pH 5 compared to SEQ ID NO:36 when determined according to Example 7 herein. In one embodiment, the polypeptide has improved endo-1,3-α-glucanase activity at pH 6 compared to SEQ ID NO:36 when determined according to Example 7 herein. In one embodiment, the polypeptide has improved endo-1,3-α-glucanase activity at pH 7 compared to SEQ ID NO:36 when determined according to Example 7 herein. In one embodiment, the polypeptide has improved endo-1,3-α-glucanase activity at pH 8 compared to SEQ ID NO:36 when determined according to Example 7 herein. In one embodiment, the polypeptide has improved endo-1,3-α-glucanase activity at pH 9 compared to SEQ ID NO:36 when determined according to Example 7 herein. In one embodiment, the polypeptide has improved endo-1,3-α-glucanase activity at pH 10 compared to SEQ ID NO:36 when determined according to Example 7 herein.

The polypeptide may be a fusion polypeptide.

In an aspect, the polypeptide is isolated.

In another aspect, the polypeptide is purified.

In an aspect, the invention relates to polypeptides having endo-1,3-α-glucanase activity selected from the group consisting of:
(a) a polypeptide having at least 60% sequence identity to SEQ ID NO:26;
(b) a polypeptide having at least 60% sequence identity to SEQ ID NO:27;
(c) a polypeptide having at least 60% sequence identity to a mature polypeptide of SEQ ID NO:26;
(d) a polypeptide encoded by a polynucleotide having at least 60% sequence identity to the mature polypeptide coding sequence of SEQ ID NO:25;
(e) a polypeptide derived from SEQ ID NO:26, a mature polypeptide of SEQ ID NO:26, or SEQ ID NO:27 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:26 or a mature polypeptide of SEQ ID NO:26.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:27.

The polypeptide preferably comprises, consists essentially of, or consists of the amino acid sequence of SEQ ID NO:29 or a mature polypeptide thereof.

The polypeptide preferably comprises, consists essentially of, or consists of the amino acid sequence of SEQ ID NO:27.

The polypeptide may have an N-terminal and/or C-terminal extension of one or more amino acids, e.g., 1-5 amino acids.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO:25.

The polynucleotide encoding the polypeptide preferably comprises, consists essentially of, or consists of nucleotides 1 to 2184 of SEQ ID NO:25, in particular nucleotides 115 to 2184 of SEQ ID NO:25.

In another aspect, the polypeptide is derived from SEQ ID NO:26 by substitution, deletion or addition of one or several amino acids. In another aspect, the polypeptide is derived from a mature polypeptide of SEQ ID NO:26 by substitution, deletion or addition of one or several amino acids. In another aspect, the polypeptide is derived from SEQ ID NO:27 by substitution, deletion or addition of one or more amino acids. In some embodiments, the polypeptide is a variant of SEQ ID NO:27 comprising a substitution, deletion, and/or insertion at one or more positions. In one aspect, the number of amino acid substitutions, deletions and/or insertions introduced into the polypeptide of SEQ ID NO:27 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding module.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability at pH 5 and/or pH 6 compared to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol

In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

In one embodiment, the polypeptide displays endo-1,3-α-glucanase activity at pH 5-10, preferably at pH 6-9, most preferably at pH 7-8. In a preferred embodiment, the polypeptide has improved endo-1,3-α-glucanase activity compared to SEQ ID NO:36 at pH 5-10, preferably at pH 6-9, most preferably at pH 7-8, when determined according to Example 7 herein. In one embodiment, the polypeptide has improved endo-1,3-α-glucanase activity at pH 5 compared to SEQ ID NO:36 when determined according to Example 7 herein. In one embodiment, the polypeptide has improved endo-1,3-α-glucanase activity at pH 6 compared to SEQ ID NO:36 when determined according to Example 7 herein. In one embodiment, the polypeptide has improved endo-1,3-α-glucanase activity at pH 7 compared to SEQ ID NO:36 when determined according to Example 7 herein. In one embodiment, the polypeptide has improved endo-1,3-α-glucanase activity at pH 8 compared to SEQ ID NO:36 when determined according to Example 7 herein. In one embodiment, the polypeptide has improved endo-1,3-α-glucanase activity at pH 9 compared to SEQ ID NO:36 when determined according to Example 7 herein. In one embodiment, the polypeptide has improved endo-1,3-α-glucanase activity at pH 10 compared to SEQ ID NO:36 when determined according to Example 7 herein.

The polypeptide may be a fusion polypeptide.

In an aspect, the polypeptide is isolated.

In another aspect, the polypeptide is purified.

In an aspect, the invention relates to polypeptides having endo-1,3-α-glucanase activity selected from the group consisting of:
(a) a polypeptide having at least 60% sequence identity to SEQ ID NO:29;
(b) a polypeptide having at least 60% sequence identity to SEQ ID NO:30;
(c) a polypeptide having at least 60% sequence identity to a mature polypeptide of SEQ ID NO:29;
(d) a polypeptide encoded by a polynucleotide having at least 60% sequence identity to the mature polypeptide coding sequence of SEQ ID NO:28;
(e) a polypeptide derived from SEQ ID NO:29, a mature polypeptide of SEQ ID NO:29, or SEQ ID NO:30 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:29 or a mature polypeptide of SEQ ID NO:29.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:30.

The polypeptide preferably comprises, consists essentially of, or consists of the amino acid sequence of SEQ ID NO:29 or a mature polypeptide thereof.

The polypeptide preferably comprises, consists essentially of, or consists of the amino acid sequence of SEQ ID NO:30.

The polypeptide may have an N-terminal and/or C-terminal extension of one or more amino acids, e.g., 1-5 amino acids.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO:28.

The polynucleotide encoding the polypeptide preferably comprises, consists essentially of, or consists of nucleotides 1 to 2028 of SEQ ID NO:28, in particular nucleotides 133 to 2028 of SEQ ID NO:28.

In another aspect, the polypeptide is derived from SEQ ID NO:29 by substitution, deletion or addition of one or several amino acids. In another aspect, the polypeptide is derived from a mature polypeptide of SEQ ID NO:29 by substitution, deletion or addition of one or several amino acids. In another aspect, the polypeptide is derived from SEQ ID NO:30 by substitution, deletion or addition of one or more amino acids. In some embodiments, the polypeptide is a variant of SEQ ID NO:30 comprising a substitution, deletion, and/or insertion at one or more positions. In one aspect, the number of amino acid substitutions, deletions and/or insertions introduced into the polypeptide of SEQ ID NO:30 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding module.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability at pH 5 and/or pH 6 compared to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol

In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

In one embodiment, the polypeptide displays endo-1,3-α-glucanase activity at pH 5-10, preferably at pH 6-9, most preferably at pH 7-8. In a preferred embodiment, the polypeptide has improved endo-1,3-α-glucanase activity compared to SEQ ID NO:36 at pH 5-10, preferably at pH 6-9, most preferably at pH 7-8, when determined according to Example 7 herein. In one embodiment, the polypeptide has improved endo-1,3-α-glucanase activity at pH 5 compared to SEQ ID NO:36 when determined according to Example 7 herein. In one embodiment, the polypeptide has improved endo-1,3-α-glucanase activity at pH 6 compared to SEQ ID NO:36 when determined according to Example 7 herein. In one embodiment, the polypeptide has improved endo-1,3-α-glucanase activity at pH 7 compared to SEQ ID NO:36 when determined according to Example 7 herein. In one embodiment, the polypeptide has improved endo-1,3-α-glucanase activity at pH 8 compared to SEQ ID NO:36 when determined according to Example 7 herein. In one embodiment, the polypeptide has improved endo-1,3-α-glucanase activity at pH 9 compared to SEQ ID NO:36 when determined according to Example 7 herein. In one embodiment, the polypeptide has improved endo-1,3-α-glucanase activity at pH 10 compared to SEQ ID NO:36 when determined according to Example 7 herein.

The polypeptide may be a fusion polypeptide.

In an aspect, the polypeptide is isolated.

In another aspect, the polypeptide is purified.

In an aspect, the invention relates to polypeptides having endo-1,3-α-glucanase activity selected from the group consisting of:
(a) a polypeptide having at least 60% sequence identity to SEQ ID NO:32;
(b) a polypeptide having at least 60% sequence identity to SEQ ID NO:33;
(c) a polypeptide having at least 60% sequence identity to a mature polypeptide of SEQ ID NO:32;
(d) a polypeptide encoded by a polynucleotide having at least 60% sequence identity to the mature polypeptide coding sequence of SEQ ID NO:31;
(e) a polypeptide derived from SEQ ID NO:32, a mature polypeptide of SEQ ID NO:32, or SEQ ID NO:33 by substitution, deletion or addition of one or several amino acids;
(f) a polypeptide derived from the polypeptide of (a), (b), (c), (d) or (e) wherein the Nand/or C-terminal end has been extended by the addition of one or more amino acids; and
(g) a fragment of the polypeptide of (a), (b), (c), (d), or (e);
   wherein the polypeptide has endo-1,3-α-glucanase activity.

In an aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:32 or a mature polypeptide of SEQ ID NO:32.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO:33.

The polypeptide preferably comprises, consists essentially of, or consists of the amino acid sequence of SEQ ID NO:32 or a mature polypeptide thereof.

The polypeptide preferably comprises, consists essentially of, or consists of the amino acid sequence of SEQ ID NO:33.

The polypeptide may have an N-terminal and/or C-terminal extension of one or more amino acids, e.g., 1-5 amino acids.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO:31.

The polynucleotide encoding the polypeptide preferably comprises, consists essentially of, or consists of nucleotides 1 to 2436 of SEQ ID NO:31, in particular nucleotides 115 to 2436 of SEQ ID NO:31.

In another aspect, the polypeptide is derived from SEQ ID NO:32 by substitution, deletion or addition of one or several amino acids. In another aspect, the polypeptide is derived from a mature polypeptide of SEQ ID NO:32 by substitution, deletion or addition of one or several amino acids. In another aspect, the polypeptide is derived from SEQ ID NO:33 by substitution, deletion or addition of one or more amino acids. In some embodiments, the polypeptide is a variant of SEQ ID NO:33 comprising a substitution, deletion, and/or insertion at one or more positions. In one aspect, the number of amino acid substitutions, deletions and/or insertions introduced into the polypeptide of SEQ ID NO:33 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding module.

In one embodiment, the polypeptide has improved thermal stability compared to SEQ ID NO: 36. Preferably, the polypeptide has improved thermal stability compared to SEQ ID NO:26 at about pH 3 to about pH 9, more preferably at about pH 4 to about pH 8, most preferably at about pH 5 to about pH 7. Preferably, the polypeptide has improved thermal stability compared to SEQ ID NO: 36 at about pH 5 and/or about pH 6, most preferably at pH 5 or pH 6, when determined according to Example 4 herein.

In one embodiment, the polypeptide has on par or improved thermal stability in the presence of at least one, e.g., at least one two, at least three, at least four, at least five, at least six, at least seven, at least eight, at least nine, or ten, oral care ingredient compared to SEQ ID NO:36, wherein the oral care ingredient is selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol. Preferably, the polypeptide has improved thermal stability in the presence of the least one oral care ingredient compared to SEQ ID NO:36 at about pH 5 and/or about pH 6, most preferably at pH 5 or pH 6, when determined according to Example 5 herein.

In one embodiment, the polypeptide has improved thermal stability at pH 5 and/or pH 6 compared to SEQ ID NO:36 when determined according to Example 5 herein in the presence of benzoate, e.g., sodium benzoate. Preferably, the polypeptide has improved thermal stability in the presence of 0.01-5% benzoate, more preferably 0.05-2.5% benzoate, even more preferably 0.1-1% benzoate, most preferably 0.1-0.5% benzoate. Preferably, the polypeptide has improved thermal stability in the presence of 1-100 mM benzoate, more preferably 5-50 mM benzoate, most preferably 10-35 mM benzoate.

In one embodiment, the polypeptide has improved thermal stability at pH 5 and/or pH 6 compared to SEQ ID NO:36 when determined according to Example 5 herein in the presence of EDTA. Preferably, them mutanase has improved thermal stability in the presence of 0.1-10 mM EDTA, more preferably 0.5-5 mM EDTA, most preferably 1 mM EDTA.

In one embodiment, the polypeptide has improved thermal stability at pH 5 and/or pH 6 compared to SEQ ID NO:36 when determined according to Example 5 herein in the presence of ethanol. Preferably, the polypeptide has improved thermal stability in the presence of 0.1-20% ethanol, more preferably 1-10% ethanol, even more preferably 2.5-7.5% ethanol, most preferably 5% ethanol. Preferably, the polypeptide has improved thermal stability in the presence of 1-100000 mM ethanol, more preferably 100-10000 mM ethanol, most preferably 1000 mM ethanol.

In one embodiment, the polypeptide has improved thermal stability at pH 5 and/or pH 6 compared to SEQ ID NO:36 when determined according to Example 5 herein in the presence of glycerol. Preferably, the polypeptide has improved thermal stability in the presence of 1-50% glycerol, more preferably 5-40% glycerol, most preferably 10-30% glycerol. Preferably, the polypeptide has improved thermal stability in the presence of 100-10000 mM glycerol, more preferably 500-5000 mM glycerol, even more preferably 750-4000 mM glycerol, most preferably 1000-3250 mM glycerol.

In one embodiment, the polypeptide has improved thermal stability at pH 5 and/or pH 6 compared to SEQ ID NO:36 when determined according to Example 5 herein in the presence of fluoride, e.g., sodium fluoride, sodium monofluorophosphate, calcium fluoride, or stannous fluoride. Preferably, the polypeptide has improved thermal stability in the presence of 1-5000 ppm fluoride, more preferably 500-2500 ppm fluoride, most preferably 1,000-1500 ppm fluoride. Preferably, the polypeptide has improved thermal stability in the presence of 1-100 mM fluoride, more preferably 5-75 mM fluoride, even more preferably 10-50 mM fluoride, most preferably 20-40 mM fluoride.

In one embodiment, the polypeptide has improved thermal stability at pH 5 and/or pH 6 compared to SEQ ID NO:36 when determined according to Example 5 herein in the presence of peroxide, e.g., hydrogen peroxide. Preferably, the polypeptide has improved thermal stability in the presence of 1-1000 mM peroxide, more preferably 50-750 mM peroxide, most preferably 100-500 mM peroxide.

In one embodiment, the polypeptide has improved thermal stability at pH 5 and/or pH 6 compared to SEQ ID NO:36 when determined according to Example 5 herein in the presence of mannitol. Preferably, the polypeptide has improved thermal stability in the presence of 1-1000 mM mannitol, more preferably 150-750 mM mannitol, most preferably 250-550 mM mannitol In one embodiment, the polypeptide has improved thermal stability at pH 5 and/or pH 6 compared to SEQ ID NO:36 when determined according to Example 5 herein in the presence of phosphate, e.g., sodium phosphate or potassium phosphate. Preferably, the polypeptide has improved thermal stability in the presence of 1-50 mM phosphate, more preferably 2.5-25 mM phosphate, even more preferably 5-10 mM phosphate.

In one embodiment, the polypeptide has improved thermal stability at pH 5 and/or pH 6 compared to SEQ ID NO:36 when determined according to Example 5 herein in the presence of sorbate, e.g., sodium sorbate, potassium sorbate, or calcium sorbate. Preferably, the polypeptide has improved thermal stability in the presence of 0.01-5% sorbate, more preferably 0.05-2.5% sorbate, even more preferably 0.1-1% sorbate, most preferably 0.1-0.5% sorbate. Preferably, the polypeptide has improved thermal stability in the presence of 1-100 mM sorbate, more preferably 5-75 mM sorbate, even more preferably 7.5-50 mM sorbate, most preferably 10-35 mM sorbate.

In one embodiment, the polypeptide has improved thermal stability at pH 5 and/or pH 6 compared to SEQ ID NO:36 when determined according to Example 5 herein in the presence of sorbitol. Preferably, the polypeptide has improved thermal stability in the presence of 0.1-70% sorbitol, more preferably 1-60% sorbitol, even more preferably 5-50% sorbitol, most preferably 10-40% sorbitol. Preferably, the polypeptide has improved thermal stability in the presence of 100-10000 mM sorbitol, more preferably 250-5000 mM sorbitol, even more preferably 500-2500 mM sorbitol, most preferably 550-2200 mM sorbitol.

In one embodiment, the polypeptide provides on par or improved, preferably improved, biofilm prevention compared to SEQ ID NO:36 when determined according to Example 6 herein. In one embodiment, the polypeptide provides on par or improved biofilm prevention at about 1 ppm, about 10 ppm, and/or about 60 ppm. In a preferred embodiment, the polypeptide provides on par or improved biofilm prevention at about 1 ppm, preferably at 1 ppm. In a preferred embodiment, the polypeptide provides on par or improved biofilm prevention at about 10 ppm, preferably at 10 ppm. In a preferred embodiment, the polypeptide provides on par or improved biofilm prevention at about 60 ppm, preferably at 60 ppm. In a preferred embodiment, the polypeptide provides on par or improved biofilm prevention compared to SEQ ID NO:36 at at least 1 ppm, e.g., at least 2 ppm, at least 3 ppm, at least 4 ppm, at least 5 ppm, at least 10 ppm, at least 15 ppm, at least 20 ppm, at least 25 ppm, at least 30 ppm, at least 40 ppm, at least 50 ppm, at least 60 ppm, at least 70 ppm, at least 80 ppm, at least 90 ppm, at least 100 ppm, or more, when determined according to Example 6 herein.

The polypeptide may be a fusion polypeptide.

In an aspect, the polypeptide is isolated.

In another aspect, the polypeptide is purified.

Essential amino acids in any polypeptide of the invention can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for endo-1,3-α-glucanase activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596:583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g., Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

### Sources of Polypeptides Having Endo-1,3-α-Glucanase Activity

A polypeptide having endo-1,3-α-glucanase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide of the invention has been inserted. In one aspect, the polypeptide obtained from a given source is secreted extracellularly.

In one aspect, the polypeptide is a polypeptide obtained from Humicola insolens.

In one aspect, the polypeptide is a polypeptide obtained from Trichoderma reesei.

In one aspect, the polypeptide is a polypeptide obtained from Talaromyces bacillisporus.

In one aspect, the polypeptide is a polypeptide obtained from Talaromyces apiculatus.

In one aspect, the polypeptide is a polypeptide obtained from Streptomyces yanii.

In one aspect, the polypeptide is a polypeptide obtained from Streptomyces alni.

In one aspect, the polypeptide is a polypeptide obtained from Oribacterium sinus.

In one aspect, the polypeptide is a polypeptide obtained from Streptomyces galbus.

In one aspect, the polypeptide is a polypeptide obtained from Streptomyces glomeratus.

In one aspect, the polypeptide is a polypeptide obtained from Tetrasphaera sp-0185.

In one aspect, the polypeptide is a polypeptide obtained from Kitasatospora phosalacinea.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

The polypeptides may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Davis et al., 2012, Basic Methods in Molecular Biology, Elsevier).

### Polynucleotides

The present invention also relates to polynucleotides encoding a polypeptide of the present invention, as described herein.

The polynucleotide may be a genomic DNA, a cDNA, a synthetic DNA, a synthetic RNA, a mRNA, or a combination thereof. The polynucleotide may be cloned from a strain of Humicola, Trichoderma, Talaromyces, Streptomyces, Oribacterium, Tetrasphaera, Kitasatospora, or a related organism and thus, for example, may be a polynucleotide sequence encoding a variant of the polypeptide of the invention.

In one embodiment the polynucleotide encoding the polypeptide of the present invention is isolated from a Humicola cell, preferably a Humicola insolens cell.

In one embodiment the polynucleotide encoding the polypeptide of the present invention is isolated from a Trichoderma cell, preferably a Trichoderma reesei cell.

In one embodiment the polynucleotide encoding the polypeptide of the present invention is isolated from a Talaromyces cell, preferably a Talaromyces bacillisporus cell or a Talaromyces apiculatus cell.

In one embodiment the polynucleotide encoding the polypeptide of the present invention is isolated from a Streptomyces cell, preferably a Streptomyces yanii cell, a Streptomyces alni cell, a Streptomyces galbus cell or a Streptomyces glomeratus cell.

In one embodiment the polynucleotide encoding the polypeptide of the present invention is isolated from an Oribacterium cell, preferably an Oribacterium sinus cell.

In one embodiment the polynucleotide encoding the polypeptide of the present invention is isolated from a Tetrasphaera cell, preferably a Tetrasphaera sp-0185 cell.

In one embodiment the polynucleotide encoding the polypeptide of the present invention is isolated from a Kitasatospora cell, preferably a Kitasatospora phosalacinea cell.

The polynucleotide may also be mutated by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2:95-107.

In an aspect, the polynucleotide is isolated.

In another aspect, the polynucleotide is purified.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. Techniques for modifying polynucleotides utilizing recombinant DNA methods are well known in the art.

### Promoters

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in Sambrook et al., 1989, Molecular Cloning: A Laboratory Manual, Cold Spring Harbor Lab., NY, Davis et al., 2012, supra, and Song et al., 2016, PLOS One 11(7): e0158447.

Examples of suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenböck, 2016, “Gene Expression Systems in Fungi: Advancements and Applications”, Fungal Biology.

For expression in a yeast host, examples of useful promoters are described by Smolke et al., 2018, “Synthetic Biology: Parts, Devices and Applications” (Chapter 6: Constitutive and Regulated Promoters in Yeast: How to Design and Make Use of Promoters in S. cerevisiae), and by Schmoll and Dattenböck, 2016, “Gene Expression Systems in Fungi: Advancements and Applications”, Fungal Biology.

### Terminators

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells may be obtained from the genes for Bacillus clausii alkaline protease (aprH), Bacillus licheniformis alpha-amylase (amyL), and Escherichia coli ribosomal RNA (rmnB).

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenböck, 2016, “Gene Expression Systems in Fungi: Advancements and Applications”, Fungal Biology.

Preferred terminators for yeast host cells may be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, Yeast 8:423-488.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177:3465-3471).

Examples of mRNA stabilizer regions for fungal cells are described in Geisberg et al., 2014, Cell 156(4): 812-824, and in Morozov et al., 2006, Eukaryotic Cell 5(11): 1838-1846.

### Leader Sequences

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Suitable leaders for bacterial host cells are described by Hambraeus et al., 2000, Microbiology 146(12): 3051-3059, and by Kaberdin and Blasi, 2006, FEMS Microbiol. Rev. 30(6): 967-979.

Preferred leaders for filamentous fungal host cells may be obtained from the genes for Aspergillus oryzae TAKA amylase and Aspergillus nidulans triose phosphate isomerase.

Suitable leaders for yeast host cells may be obtained from the genes for Saccharomyces cerevisiae enolase (ENO-1), Saccharomyces cerevisiae 3-phosphoglycerate kinase, alcohol Saccharomyces cerevisiae alpha-factor, and Saccharomyces cerevisiae dehydrogenase/glyceraldehyde-3-phosphate dehydrogenase (ADH2/GAP).

### Polyadenylation Sequences

The control sequence may also be a polyadenylation sequence, a sequence operably linked to the 3′-terminus of the polynucleotide which, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15:5983-5990.

### Signal Peptides

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. Any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alphaamylase, Bacillus stearothermophilus neutral proteases (nprT, nprS, nprM), and Bacillus subtilis prsA. Further signal peptides are described by Freudl, 2018, Microbial Cell Factories 17:52.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40:949-955 Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

### Propeptides

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence. Additionally or alternatively, when both signal peptide and propeptide sequences are present, the polypeptide may comprise only a part of the signal peptide sequence and/or only a part of the propeptide sequence. Alternatively, the final or isolated polypeptide may comprise a mixture of mature polypeptides and polypeptides which comprise, either partly or in full length, a propeptide sequence and/or a signal peptide sequence.

### Regulatory Sequences

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In fungal systems, these regulatory sequences include the dihydrofolate reductase gene that is amplified in the presence of methotrexate, and the metallothionein genes that are amplified with heavy metals.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52:7-23, as well in Balleza et al., 2009, FEMS Microbiol. Rev. 33(1): 133-151.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or nonhomologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

The present invention also relates to recombinant host cells, comprising a polynucleotide of the present invention operably linked to one or more control sequences that direct the production of a polypeptide of the present invention.

A construct or vector comprising a polynucleotide is introduced into a host cell so that the construct or vector is maintained as a chromosomal integrant or as a self-replicating extrachromosomal vector as described earlier. The choice of a host cell will to a large extent depend upon the gene encoding the polypeptide and its source. The polypeptide can be native or heterologous to the recombinant host cell. Also, at least one of the one or more control sequences can be heterologous to the polynucleotide encoding the polypeptide. The recombinant host cell may comprise a single copy, or at least two copies, e.g., three, four, five, or more copies of the polynucleotide of the present invention.

The host cell may be any microbial cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis and Bacillus subtilis cell.

For purposes of this invention, Bacillus classes/genera/species shall be defined as described in Patel and Gupta, 2020, Int. J. Syst. Evol. Microbiol. 70:406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98:6289-6294, Choi et al., 2006, J. Microbiol. Methods 64:391-397, and Donald et al., 2013, J. Bacteriol. 195(11): 2612-2620.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16:168 and procedures described in EP 238023, Yelton et al., 1984, Proc. Natl. Acad. Sci. USA 81:1470-1474, Christensen et al., 1988, Bio/Technology 6:1419-1422, and Lubertozzi and Keasling, 2009, Biotechn. Advances 27:53-75. However, any method known in the art for introducing DNA into a fungal host cell can be used, and the DNA can be introduced as linearized or as circular polynucleotide.

The fungal host cell may be a yeast cell. “Yeast” as used herein includes ascosporogenous yeast (Endomycetales), basidiosporogenous yeast, and yeast belonging to the Fungi Imperfecti (Blastomycetes). For purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. In a preferred embodiment, the yeast host cell is a Pichia or Komagataella cell, e.g., a Pichia pastoris cell (Komagataella phaffii).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In an aspect, the host cell is isolated.

In another aspect, the host cell is purified.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide.

In one aspect, the cell is a Humicola cell, preferably a Humicola insolens cell.

In one aspect, the cell is a Trichoderma cell, preferably a Trichoderma reesei cell.

In one aspect, the cell is a Talaromyces cell, preferably a Talaromyces bacillisporus cell or a Talaromyces apiculatus cell.

In one aspect, the cell is a Streptomyces cell, preferably a Streptomyces yanii cell, a Streptomyces alni cell, a Streptomyces galbus cell or a Streptomyces glomeratus cell.

In one aspect, the cell is an Oribacterium cell, preferably an Oribacterium sinus cell.

In one aspect, the cell is a Tetrasphaera cell, preferably a Tetrasphaera sp-0185 cell.

In one aspect, the cell is a Kitasatospora cell, preferably a Kitasatospora phosalacinea cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cell is cultivated in a nutrient medium suitable for production of the polypeptide using methods known in the art. For example, the cell may be cultivated by shake flask cultivation, or small-scale or large-scale fermentation (including continuous, batch, fed-batch, or solid-state, and/or microcarrier-based fermentations) in laboratory or industrial fermentors in a suitable medium and under conditions allowing the polypeptide to be expressed and/or isolated. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptide, including, but not limited to, the use of specific antibodies, formation of an enzyme product, disappearance of an enzyme substrate, or an assay determining the relative or specific activity of the polypeptide.

The polypeptide may be recovered from the medium using methods known in the art, including, but not limited to, collection, centrifugation, filtration, extraction, spray-drying, evaporation, or precipitation. In one aspect, a whole fermentation broth comprising the polypeptide is recovered. In another aspect, a cell-free fermentation broth comprising the polypeptide is recovered.

The polypeptide may be purified by a variety of procedures known in the art to obtain substantially pure polypeptides and/or polypeptide fragments (see, e.g., Wingfield, 2015, Current Protocols in Protein Science; 80(1): 6.1.1-6.1.35; Labrou, 2014, Protein Downstream Processing, 1129:3-10).

In an alternative aspect, the polypeptide is not recovered.

### Mutanase Granules

The present invention also relates to enzyme granules/particles comprising a polypeptide of the invention. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm.

The core diameter, measured as equivalent spherical diameter, can be determined using laser diffraction, such as using a Malvern Mastersizer and/or the method described under ISO13320 (2020).

In an embodiment, the core comprises a polypeptide having endo-1,3-α-glucanase activity of the present invention.

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the polypeptide absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100%, 70%, 50%, 40% or 30%.

The coating is preferably at least 0.1 μm thick, particularly at least 0.5 μm, at least 1 μm or at least 5 μm. In some embodiments, the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit has few or no uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e., a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄·7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Vol. 1; 1980; Elsevier. Preparation methods include known feed and granule formulation technologies, e.g.,

(a) Spray dried products, wherein a liquid polypeptide-containing solution is atomized in a spray drying tower to form small droplets which during their way down the drying tower dry to form a polypeptide-containing particulate material. Very small particles can be produced this way (Michael S. Showell (editor); Powdered detergents; Surfactant Science Series; 1998; Vol. 71; pages 140-142; Marcel Dekker).

(b) Layered products, wherein the polypeptide is coated as a layer around a pre-formed inert core particle, wherein a polypeptide-containing solution is atomized, typically in a fluid bed apparatus wherein the pre-formed core particles are fluidized, and the polypeptide-containing solution adheres to the core particles and dries up to leave a layer of dry polypeptide on the surface of the core particle. Particles of a desired size can be obtained this way if a useful core particle of the desired size can be found. This type of product is described in, e.g., WO 97/23606.

(c) Absorbed core particles, wherein rather than coating the polypeptide as a layer around the core, the polypeptide is absorbed onto and/or into the surface of the core. Such a process is described in WO 97/39116.

(d) Extrusion or pelletized products, wherein a polypeptide-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the polypeptide paste, which is harmful to the polypeptide (Michael S. Showell (editor); Powdered detergents; Surfactant Science Series; 1998; Vol. 71; pages 140-142; Marcel Dekker).

(e) Prilled products, wherein a polypeptide-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; Vol. 71; pages 140-142; Marcel Dekker). The product obtained is one wherein the polypeptide is uniformly distributed throughout an inert material instead of being concentrated on its surface. U.S. Pat. Nos. 4,016,040 and 4,713,245 describe this technique.

(f) Mixer granulation products, wherein a polypeptide-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the polypeptide. Such a process is described in U.S. Pat. No. 4,106,991, EP 170360, EP 304332, EP 304331, WO 90/09440 and WO 90/09428. In a particular aspect of this process, various high-shear mixers can be used as granulators. Granulates consisting of polypeptide, fillers and binders etc. are mixed with cellulose fibers to reinforce the particles to produce a so-called T-granulate. Reinforced particles are more robust, and release less enzymatic dust.

(g) Size reduction, wherein the cores are produced by milling or crushing of larger particles, pellets, tablets, briquettes etc. containing the polypeptide. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.

(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.

(i) The cores may be subjected to drying, such as in a fluid bed drier. Other known methods for drying granules in the feed or enzyme industry can be used by the skilled person. The drying preferably takes place at a product temperature of from 25 to 90° C. For some polypeptides, it is important the cores comprising the polypeptide contain a low amount of water before coating with the salt. If water sensitive polypeptides are coated with a salt before excessive water is removed, the excessive water will be trapped within the core and may affect the activity of the polypeptide negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further comprise one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, betaglucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of polypeptides by the use of co-granulates is disclosed in WO 2013/188331.

The present invention also relates to protected polypeptides prepared according to the method disclosed in EP 238216.

### Liquid Formulations

The present invention also relates to liquid compositions comprising a polypeptide of the invention. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to, water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the liquid formulation comprises 20-80% w/w of polyol. In one embodiment, the liquid formulation comprises 0.001-2% w/w preservative.

In another embodiment, the invention relates to liquid formulations comprising:
(A) 0.001-25% w/w of a polypeptide having endo-1,3-α-glucanase activity of the present invention;
(B) 20-80% w/w of polyol;
(C) optionally 0.001-2% w/w preservative; and
(D) water.

In another embodiment, the invention relates to liquid formulations comprising:
(A) 0.001-25% w/w of a polypeptide having endo-1,3-α-glucanase activity of the present invention;
(B) 0.001-2% w/w preservative;
(C) optionally 20-80% w/w of polyol; and
(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20-80% polyol (i.e., total amount of polyol), e.g., 25-75% polyol, 30-70% polyol, 35-65% polyol, or 40-60% polyol. In one embodiment, the liquid formulation comprises 20-80% polyol, e.g., 25-75% polyol, 30-70% polyol, 35-65% polyol, or 40-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20-80% polyol (i.e., total amount of polyol), e.g., 25-75% polyol, 30-70% polyol, 35-65% polyol, or 40-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02-1.5% w/w preservative, e.g., 0.05-1% w/w preservative or 0.1-0.5% w/w preservative. In one embodiment, the liquid formulation comprises 0.001-2% w/w preservative (i.e., total amount of preservative), e.g., 0.02-1.5% w/w preservative, 0.05-1% w/w preservative, or 0.1-0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, betagalactosidase, beta-glucanase, beta-glucosidase, lysophospholipase, lysozyme, alphamannosidase, beta-mannosidase (mannanase), phytase, phospholipase A1, phospholipase A2, phospholipase D, protease, pullulanase, pectin esterase, triacylglycerol lipase, xylanase, betaxylosidase or any combination thereof.

### Signal Peptide

In one aspect, the present invention also relates to a polynucleotide encoding a signal peptide comprising or consisting of amino acids 1 to 20 of SEQ ID NO:2.

In one aspect, the present invention also relates to a polynucleotide encoding a signal peptide comprising or consisting of amino acids 1 to 24 of SEQ ID NO:5.

In one aspect, the present invention also relates to a polynucleotide encoding a signal peptide comprising or consisting of amino acids 1 to 20 of SEQ ID NO:8.

In one aspect, the present invention also relates to a polynucleotide encoding a signal peptide comprising or consisting of amino acids 1 to 20 of SEQ ID NO: 11.

In one aspect, the present invention also relates to a polynucleotide encoding a signal peptide comprising or consisting of amino acids 1 to 41 of SEQ ID NO: 14.

In one aspect, the present invention also relates to a polynucleotide encoding a signal peptide comprising or consisting of amino acids 1 to 25 of SEQ ID NO: 17.

In one aspect, the present invention also relates to a polynucleotide encoding a signal peptide comprising or consisting of amino acids 1 to 26 of SEQ ID NO:20.

In one aspect, the present invention also relates to a polynucleotide encoding a signal peptide comprising or consisting of amino acids 1 to 26 of SEQ ID NO:23.

In one aspect, the present invention also relates to a polynucleotide encoding a signal peptide comprising or consisting of amino acids 1 to 38 of SEQ ID NO:26.

In one aspect, the present invention also relates to a polynucleotide encoding a signal peptide comprising or consisting of amino acids 1 to 24 of SEQ ID NO:29.

In one aspect, the present invention also relates to a polynucleotide encoding a signal peptide comprising or consisting of amino acids 1 to 38 of SEQ ID NO:32.

The polynucleotides may further comprise a gene encoding a protein, which is operably linked to the signal peptide. The protein is preferably heterologous to the signal peptide. In one aspect, the polynucleotide encoding the signal peptide is nucleotides 1 to 60 of SEQ ID NO: 1. In one aspect, the polynucleotide encoding the signal peptide is nucleotides 1 to 72 of SEQ ID NO:4. In one aspect, the polynucleotide encoding the signal peptide is nucleotides 1 to 60 of SEQ ID NO: 7. In one aspect, the polynucleotide encoding the signal peptide is nucleotides 1 to 60 of SEQ ID NO: 10. In one aspect, the polynucleotide encoding the signal peptide is nucleotides 1 to 123 of SEQ ID NO: 13. In one aspect, the polynucleotide encoding the signal peptide is nucleotides 1 to 75 of SEQ ID NO:16. In one aspect, the polynucleotide encoding the signal peptide is nucleotides 1 to 78 of SEQ ID NO: 19. In one aspect, the polynucleotide encoding the signal peptide is nucleotides 1 to 78 of SEQ ID NO:22. In one aspect, the polynucleotide encoding the signal peptide is nucleotides 1 to 114 of SEQ ID NO:25. In one aspect, the polynucleotide encoding the signal peptide is nucleotides 1 to 132 of SEQ ID NO:28. In one aspect, the polynucleotide encoding the signal peptide is nucleotides 1 to 114 of SEQ ID NO:31.

The present invention also relates to nucleic acid constructs, expression vectors and recombinant host cells comprising such polynucleotides.

The present invention also relates to methods of producing a protein, comprising (a) cultivating a recombinant host cell comprising such polynucleotide; and optionally (b) recovering the protein.

The protein may be native or heterologous to a host cell. The term “protein” is not meant herein to refer to a specific length of the encoded product and, therefore, encompasses peptides, oligopeptides, and polypeptides. The term “protein” also encompasses two or more polypeptides combined to form the encoded product. The proteins also include hybrid polypeptides and fusion polypeptides.

Preferably, the protein is a hormone, enzyme, receptor or portion thereof, antibody or portion thereof, or reporter. For example, the protein may be a hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The gene may be obtained from any prokaryotic, eukaryotic, or other source.

In one aspect, the present invention relates to a polynucleotide encoding a signal peptide selected from the group consisting of:
(a) a signal peptide comprising or consisting of amino acids 1 to 20 of SEQ ID NO: 2,
(b) a signal peptide comprising or consisting of amino acids 1 to 24 of SEQ ID NO: 5,
(c) a signal peptide comprising or consisting of amino acids 1 to 20 of SEQ ID NO: 8,
(d) a signal peptide comprising or consisting of amino acids 1 to 20 of SEQ ID NO: 11,
(e) a signal peptide comprising or consisting of amino acids 1 to 41 of SEQ ID NO: 14,
(f) a signal peptide comprising or consisting of amino acids 1 to 25 of SEQ ID NO: 17,
(g) a signal peptide comprising or consisting of amino acids 1 to 26 of SEQ ID NO: 20,
(h) a signal peptide comprising or consisting of amino acids 1 to 26 of SEQ ID NO: 23,
(i) a signal peptide comprising or consisting of amino acids 1 to 38 of SEQ ID NO: 26,
(j) a signal peptide comprising or consisting of amino acids 1 to 24 of SEQ ID NO: 29, and
(k) a signal peptide comprising or consisting of amino acids 1 to 38 of SEQ ID NO: 32;
   wherein the polynucleotide encoding a signal peptide is operably linked to a polynucleotide encoding a polypeptide which is heterologous to the signal peptide.

The invention also relates to a nucleic acid construct or an expression vector comprising the polynucleotide encoding a signal peptide of the invention operably linked to a polynucleotide encoding a polypeptide which is heterologous to the signal peptide. The invention also relates to a recombinant host cell comprising said nucleic acid construct or expression. The invention also relates to a method of producing a polypeptide, the method comprising cultivating said recombinant host cell under conditions conducive for production of the protein, and, optionally, recovering the polypeptide.

### Oral Care Ingredients and Formats

In one aspect, the present invention also relates to enzymatic oral care compositions comprising a polypeptide of the invention and at least one oral care ingredient.

Thus, in an aspect, the present invention relates to an oral care composition comprising a mutanase selected from the group consisting of:
(a) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:3;
(b) a polypeptide having at least 90%, e.g., at least 90%, at least 91%, at least 92%, at least 93%, at least 94%, at least 95%, at least 96%, at least 97%, at least 98%, at least 99%, or 100%, sequence identity to SEQ ID NO:6;
(c) a polypeptide having at least 65%, e.g., at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:9;
(d) a polypeptide having at least 65%, e.g., at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO: 12;
(e) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO: 15;
(f) a polypeptide having at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO: 18;
(g) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:21;
(h) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:24;
(i) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:27;
(j) a polypeptide having at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:30; and
(k) a polypeptide having at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:33;

wherein the polypeptide has endo-1,3-α-glucanase activity, and wherein the oral care composition further comprises at least one oral care ingredient.

Preferably, the mutanase is selected from the group consisting of:
(a) a polypeptide comprising, consisting essentially of, or consisting of SEQ ID NO:3;
(b) a polypeptide comprising, consisting essentially of, or consisting of SEQ ID NO:6;
(c) a polypeptide comprising, consisting essentially of, or consisting of SEQ ID NO:9;
(d) a polypeptide comprising, consisting essentially of, or consisting of SEQ ID NO: 12;
(e) a polypeptide comprising, consisting essentially of, or consisting of SEQ ID NO: 15;
(f) a polypeptide comprising, consisting essentially of, or consisting of SEQ ID NO: 18;
(g) a polypeptide comprising, consisting essentially of, or consisting of SEQ ID NO:21;
(h) a polypeptide comprising, consisting essentially of, or consisting of SEQ ID NO:24;
(i) a polypeptide comprising, consisting essentially of, or consisting of SEQ ID NO:27;
(j) a polypeptide comprising, consisting essentially of, or consisting of SEQ ID NO:30; and
(k) a polypeptide comprising, consisting essentially of, or consisting of SEQ ID NO:33;

The at least one oral care ingredient may be varied according to the type of oral care composition as well as the desired characteristics and/or activities of the oral care compositions. For the purpose of the present invention, the terms “ingredient” and “component” are used interchangeably in relation to oral care compositions.

In a preferred embodiment, the at least one oral care ingredient is selected from the group consisting of abrasives, humectants, solvents, thickening agents, binding agents, buffering agents, foaming agents, foaming modulators, sweetening agents, softening agents, plasticizing agents, flavoring agents, coloring agents, therapeutic agents, anti-microbial agents, tartar-controlling agents, fluoride sources, preservatives, detergents, surfactants, coloring agents, buffering agents, softeners, plasticizers, whitening agents, bleaching agents, gum-base ingredients, and bulking agents.

An oral care composition of the invention may be an internal oral care composition such as toothpaste or toothpaste tablet, dental cream, mouthwash or mouthwash tablet, mouth rinse, lozenges, pastilles, chewing gum, confectionary, candy, and the like, which is designed to remove biofilm inside the oral cavity, e.g., biofilm residing on teeth, on soft tissues of the oral cavity, and on dentures residing in the oral cavity.

In one aspect, the oral care composition is a toothpaste.

In one aspect, the oral care composition is a mouthwash, mouthwash tablet, or a mouth rinse, preferably a mouthwash.

In one aspect, the oral care composition is a lozenge.

In one aspect, the oral care composition is a chewing gum.

An oral care composition of the invention may also be an external oral care composition such as denture cleaning solution, denture cleaning tablet, denture cleaning powder, and the like, which is designed to remove biofilm from dentures that have been removed from the oral cavity for cleaning.

In a preferred embodiment, the oral care composition is an internal oral care composition, and the at least one oral care ingredient is selected from the group consisting of abrasives, humectants, solvents, thickening agents, binding agents, buffering agents, foaming agents, foaming modulators, sweetening agents, softening agents, plasticizing agents, flavoring agents, coloring agents, therapeutic agents, anti-microbial agents, tartar-controlling agents, fluoride sources, preservatives, detergents, surfactants, coloring agents, buffering agents, softeners, plasticizers, whitening agents, bleaching agents, gum-base ingredients, and bulking agents.

Although the oral care ingredients mentioned herein are categorized by a general header according to a functionality, this is not to be construed as a limitation, as an ingredient may comprise additional functionalities as will be appreciated by the skilled person.

### Toothpaste, Dental Cream, Mouthwash, and Mouth Rinse

Internal oral care compositions of the invention in the form of toothpaste or toothpaste tablet, dental cream, mouthwash or mouthwash tablet, and mouth rinse may include oral care ingredients selected from the following categories:

| Type of ingredient | Examples | Amount (weight %) |
|---|---|---|
| Abrasives | Calcium Carbonate, Sodium bicarbonate, | 0 to 70% |
| | Diatomite, Charcoal, Dicalcium phosphate | |
| | dehydrate, Hydrated Silica, Hydrated Alumina | |
| Humectants | Glycerol, sorbitol and propylene glycol | 0 to 80% |
| Solvents | Water, ethanol, water/ethanol mixtures | 0.001 to 95% |
| Thickening/binding | Xanthan gum, cellulose gum (sodium | 0.01 to 20% |
| agents | carboxymethyl cellulose), hydroxyethyl cellulose, | |
| | carrageenan, glycerin, potassium thiocyanate, | |
| | PVM/MA copolymer, carbomer, arrowroot | |
| | powder | |
| Buffering agents | Sodium Hydroxide, Sodium phosphate, Di- and | 0.001 to 10% |
| | trisodium phosphate, Sodium Citrate, Calcium | |
| | Glycerophosphate, | |
| Formulation and | Sodium lauryl sulfate (SLS), sodium lauryl | 0.001 to 10% |
| foaming agents | sarcosinate, sodium lauroyl glutamate, sodium | |
| (detergents, | methyl cocoyl taurate, Steareth-30, coco-glucoside, | |
| surfactants) | propylene glycol (1,2-propanediol), | |
| | Poloxamer 407, Polysorbate 20, Polysorbate 80 | |
| Foaming | PEG's (polyethylene glycols) | 0.001 to 10% |
| agents/modulators | | |
| Sweetening agents | Xylitol, Sorbitol, Erythritol, mannitol, Sodium | 0.001 to 60% |
| | Saccharin, Maltitol, Stevia, Sucralose, | |
| | Acesulfame K, aspartame, Hydrogenated Starch | |
| | Hydrolysate | |
| Softener/plasticizer | Lecithin, Hydrogenated vegetable oils, Glycerol | 0.001 to 5% |
| | ester, Lanolin, Methyl ester, Pentaerythritol | |
| | ester, Stearic Acid, sodium and Potassium | |
| | Stearates | |
| Flavoring agents | Peppermint oil, spearmint oil | 0.001 to 5% |
| Coloring agents | Titanium dioxide, beta-carotene, | 0.001 to 20% |
| | FD&C Blue 1 Aluminum Lake, FD&C Blue 2 | |
| | Lake, FD&C Yellow 5 Aluminum Lake, FD&C | |
| | Yellow 5 | |
| Preservatives | Sodium Benzoate, Methyl Paraben, Ethyl | 0.001 to 30% |
| | Paraben, Citric acid, Potassium Sorbate, Calcium | |
| | Citrate, Propylene glycol | |
| Whitening/bleaching | Hydrogen peroxide, sodium hypochlorite, | 0.01 to 10% |
| agents | sodium perborate | |
| Therapeutic agents | Calcium Fluoride, Sodium Fluoride, Triclosan, | 0.001 to 50% |
| (including anti- | Alcohol, various essential oils (Thymol, | |
| bacterial, anti-carries, | Menthol), Benzoic acid, Sodium benzoate, zinc | |
| anti-inflammatory | Gluconate, Aloe Vera, chloride, cetylpyridinuim | |
| agents) | chloride, Domiphen bromide, Methyl salicylate, | |
| | Chlorhexidine digluconate | |

### Toothpaste

Toothpastes and dental creams/gels typically include abrasives, solvents, humectants, detergents/surfactants, thickening and binding agents, buffering agents, flavoring agents, sweetening agents, fluoride sources, therapeutic agents, enzymes, coloring agents, and preservatives.

In a preferred embodiment, the present invention relates to oral care compositions in the form of a toothpaste or dental cream comprising a mutanase of the invention and at least one oral care ingredient, wherein the at least one oral care ingredient is selected from the following ingredients:

| Type of ingredient | Examples | Amount (weight %) |
|---|---|---|
| Abrasive | Calcium carbonate, sodium bicarbonate, | 10 to 50% |
| | hydrated silica, hydrated Alumina | |
| Solvent | Water | 5 to 40% |
| Humectant | Glycerin and sorbitol | 20 to 70% |
| Detergent/Surfactant | Sodium lauryl sulphate (SLS), sodium | 1 to 3% |
| | lauryl sarcosinate, cocamidopopybetaine | |
| | and steareth-30 | |
| Thickening and binding | Natural gums (e.g. xanthan gum, | 1 to 2% |
| agent | cellulose gum) or synthetic polymers (e.g., | |
| | PVM/MA copolymer, carbomer). | |
| | hydroxyethyl cellulose, carrageenan, glycerin. | |
| Buffering agent | Sodium citrate, calcium glycerophosphate, | 1 to 60% |
| | sodium hydroxide, di- and | |
| | trisodium pyrophosphate, disodium | |
| | phosphate | |
| Flavoring agent | Peppermint oil, spearmint oil | 0 to 2% |
| Sweetening agent | Xylitol, sorbitol, erythritol, saccharin, | 0 to 2% |
| | stevia, sucralose | |
| Fluoride source | Sodium fluoride, calcium fluoride, | 0.001 to 10% |
| | stannous fluoride, sodium | |
| | monofluorophosphate | |
| Therapeutic agent | Triclosan, zinc gluconate, aloe vera | 0.001 to 50% |
| | extracts, chloride, cetylpyridinuim chloride, | |
| | domiphen bromide, methyl salicylate, | |
| | chlorhexidine, chlorhexidine digluconate | |
| Coloring agent | Titanium dioxide | 0 to 0.5% |
| Preservative | Sodium gluconate, citric acid, sodium | 0.05 to 0.5% |
| | benzoate | |

An oral care composition of the invention may be a toothpaste comprising the following ingredients (in weight % of the final toothpaste composition):
Abrasive: 10 to 70%
Humectant: 0 to 80%
Thickening agent: 0.1 to 20%
Binding agent: 0.01 to 10%
Sweetening agent: 0.1 to 5%
Foaming agent: 0 to 15%
Mutanase: 0.01 to 10%

### Mouthwash

Mouthwashes and mouth rinses of the invention, including plaque removing liquids, typically comprise a mutanase of the invention, a carrier liquid, detergents/surfactants, buffering agents, flavoring agents, humectants, sweetening agents, therapeutic agents, fluoride sources, coloring agents, and preservatives.

In a preferred embodiment, the present invention relates to oral care compositions in the form of a mouthwash or mouth rinse comprising a mutanase of the invention and at least one oral care ingredient, wherein the at least one oral care ingredient is selected from the following ingredients:

| Type of ingredient | Example |
|---|---|
| Carrier liquid, | Water, ethanol (18-27%), water/ethanol |
| usually 60-99% | mixture |
| Detergent/surfactant | Poloxamer 407, Polysorbate 20, Polysorbate |
| | 80, Steareth-30 |
| Buffering agent | Sodium hydroxide, (Di)-sodium phosphate, |
| | sodium citrate, benzoic acid, sodium benzoate |
| Flavoring agent | Peppermint oil, spearmint oil |
| Humectants | Glycerin, sorbitol |
| Sweetening agents | Saccharin, xylitol |
| Therapeutic agent | Triclosan, zinc gluconate, aloe vera extracts, |
| | chloride, cetylpyridinuim chloride, domiphen |
| | bromide, methyl salicylate, chlorhexidine, |
| | chlorhexidine digluconate |
| Fluoride source | Sodium fluoride, calcium fluoride, stannous |
| | fluoride, or sodium monofluorophosphate |
| Coloring agent | Titanium oxide |
| Preservative | Sodium Benzoate, benzoic acid, citric acid, |
| | propylene glycol |

An oral care composition of the invention may be a mouthwash comprising the following ingredients (in weight % of the final mouthwash composition):
Water: 0 to 70%
Ethanol: 0 to 20%
Humectant: 0 to 20%
Surfactant: 0 to 2%
Mutanase: 0.01 to 10%
Other ingredients: 0 to 2% (e.g., flavors, sweeteners, fluoride sources).

The mouthwash composition may be buffered with an appropriate buffer, e.g., sodium citrate or phosphate in the pH range 6-7.5.

Relevant oral care ingredients suitable for toothpastes, dental creams, mouthwashes, and mouth rinses is further detailed below. The skilled person is capable of varying the oral care ingredients according to the type of oral care composition as well as the desired characteristics and/or activities of the specific oral care composition. An oral care composition may not necessarily comprise all of the mentioned ingredients.

### Abrasives

Abrasive polishing material might be incorporated into the oral care composition of the invention. According to the invention said abrasive polishing material includes alumina and hydrates thereof, such as alpha alumina trihydrate, magnesium trisilicate, magnesium carbonate, kaolin, aluminosilicates, such as calcined aluminum silicate and aluminum silicate, calcium carbonate, zirconium silicate, bentonite, silicium dioxide, sodium bicarbonate, and also powdered plastics, such as polyvinyl chloride, polyamides, polymethyl methacrylate, polystyrene, phenol-formaldehyde resins, melamine-formaldehyde resins, urea-formaldehyde resins, epoxy resins, powdered polyethylene, silica xerogels, hydrogels and aerogels, and the like.

Also suitable as abrasive agents are calcium pyrophosphate, water-insoluble alkali metaphosphates, poly-metaphosphates, dicalcium phosphate and/or its dihydrate, dicalcium orthophosphate, tricalcium phosphate, particulate hydroxyapatite, and the like. It is also possible to employ mixtures of these substances.

Silica dental abrasives of various types are preferred because of their unique benefits of exceptional dental cleaning and polishing performance without unduly abrading tooth enamel or dentine and which have a good compatibility with other possible ingredients, like metal ions and fluoride.

Dependent on the oral care composition, the abrasive product may be present in from 0 to 70% by weight, preferably from 1% to 70%.

For toothpastes the abrasive material content typically lies in the range of from 10% to 70% by weight of the final tooth-paste product.

### Humectants

Humectants are employed to prevent loss of water from, e.g., toothpastes and to avoid hardening of toothpastes upon exposure to air. Some humectants also give a desirable sweetness of flavor to toothpaste and mouthwash compositions. Suitable humectants for use in oral care compositions according to the invention include the following compounds and mixtures thereof: glycerol, polyol, sorbitol, xylitol, maltitol, lactitol, polyoxyethylene, polyethylene glycols (PEG), polypropylene glycols, propylene glycol, 1,3-propanediol, 1,4-butanediol, hydrogenated partially hydrolyzed polysaccharides and the like, coconut fatty acid, amide of N-methyl-taurine, and Pluronic®.

Humectants are in generally present in from 0% to 80%, preferably 5 to 70% by weight.

### Thickening/Binding Agents

Suitable thickening and/or binding agents include silica, starch, tragacanth gum, xanthan gum, karaya gum, carrageenans (extracts of Irish moss), gum arabic, alginates, pectin, cellulose derivatives, such as hydroxyethyl cellulose, sodium carboxymethyl cellulose, hydroxypropyl cellulose and hydroxyethyl propyl cellulose, polyacrylic acid and its salts, polyvinylpyrrolidone and carboxyvinyl polymers, as well as inorganic thickeners such as amorphous silica compounds. These agents stabilize the oral care compositions of the invention.

Thickeners may be present in toothpaste, dental creams and gels as well as in mouthwashes in an amount of from 0.1 to 20% by weight, and binders to the extent of from 0.01 to 10% by weight of the final product.

### Foaming Agents and Foaming Modulators

As foaming agent soap, anionic, cationic, non-ionic, amphoteric and/or zwitterionic surfactants can be used, either alone or in combinations. These may be present at levels of from 0% to 15%, preferably from 0.1% to 13%, more preferably from 0.25% to 10% by weight of the final product. Surfactants are only suitable to the extent that they do not exert an inactivation effect on the enzymes and other components included in the oral care composition. Useful surface-active agents include anionic, nonionic, and ampholytic compounds, with anionic compounds being preferred.

Examples of suitable surfactants include salts of the higher alkyl sulfates, such as sodium lauryl sulfate or other suitable alkyl sulfates having 8 to 18 carbon atoms in the alkyl group; sodium lauryl sulfoacetate, salts of sulfonated monoglycerides of higher fatty acids, such as sodium coconut monoglyceride sulfonate or other suitable sulfonated monoglycerides of fatty acids of 10 to 18 carbon atoms; salts of amides of higher fatty acid, e.g., 12 to 16 carbon atom acids, with lower aliphatic amino acids, such as sodium-N-methyl-N-palmitoyl tauride, sodium N-lauroyl-, N-myristoyl- and N-palmitoyl sarcosinates; salts of the esters of such fatty acids with isotopic acid or with glycerol monosulfate; such as the sodium salt of monosulfated monoglyceride of hydrogenated coconut oil fatty acids; salts of olefin sulfonates, e.g., alkene sulfonates or alkene sulfonates or mixtures thereof having 12 to 16 carbon atoms in the carbon chain of the molecule; and soaps of higher fatty acids, such as those of 12 to 18 carbon atoms, e.g., coconut fatty acids.

The cation of the salt may be sodium, potassium or mono-, di or triethanol amine. The nonionic surfactants include sucrose/fatty acid esters, maltose/fatty acid esters, maltitol/fatty acid esters, maltotriitol/fatty acid esters, maltotetraitol/fatty acid esters, maltopentaitol/fatty acid esters, maltohexaitol/fatty acid esters, mahoheptaitol/fatty acid esters, sorbitan/fatty acid esters, lactose/fatty acid esters, lactinose/fatty acid esters, polyoxyethylene/polyoxypropylene copolymers, polyoxyethylene alkyl ethers, polyoxyethylene/fatty acid esters, fatty acid alkanolamides, polyoxyethylene sorbitan/fatty acid esters, polyoxyethylene/hydrogenated castor oil, and polyglycerin/fatty acid esters.

Most preferred are sodium lauryl sulphate, sodium dodecylbenzene sulphonate and sodium lauryl sarcosinate.

Preferred foaming modulators include polyethylene glycols.

Foaming agents and foaming modulators may be present from in an amount of from 0% to 15% by weight, preferably from 0.01% to 10% by weight.

### Sweetening Agents

Suitable sweeteners include, but are not limited to, saccharin and water-soluble salts thereof, dextrose, sucrose, lactose, maltose, levulose, aspartame, cyclamate salts, D-tryptophan, dihydrochalchones, acesulphame, stevioside, levaudioside, glycyrrhizins, pellartine, thaumatin, p-methoxycinnamic aldehyde, hydrogenated starch hydrolysates, xylitol, sorbitol, erythritol, mannitol, and mixtures thereof.

Sweeteners may be present from in an amount of from 0.001% to 60% by weight, preferably from 0.01% to 50% by weight.

### Flavoring Agents

Flavoring agents are usually present in low amounts, such as from 0.01% to about 5% by weight, especially from 0.1% to 5%. The flavors that may be used in the invention include, but are not limited to, wintergreen oil, peppermint oil, spearmint oil, clove bud oil, menthol, anethole, methyl salicylate, eucalyptol, cassia, 1-inenthyl acetate, sage, eugenol, parsley oil, oxanone, alpha-irisone, marjoram, lemon, orange, cranberry, propenyl guaethol, cinnamon, vanillin, ethyl vanillin, heliotropine, 4-cis-heptenal, diacetyl, methylpara-tert-butyl phenyl acetate, carvone, cineole, menthone, cinnamic aldehyde, limonene, ocimene, n-decyl alcohol, citronellol, alpha-terpineol, methyl acetate, citronellyl acetate, methyl eugenol, linalool, thymol, rosemary oil, pimento oil, diatomaceous oil, eucalyptus oil, and mixtures thereof.

Coolants may also be part of the flavor system or added separately to the composition. Preferred coolants in the present compositions are the paramenthan carboxyamide agents such as N-ethyl-p-menthan-3-carboxamide (known commercially as ‘WS-3”), menthol, 3-1-menthoxypropanc-1,2-diol (“TK-10”), menthone glycerol acetal (“MGA”), menthyl lactate and mixtures thereof.

### Whitening/Bleaching Agents

Whitening/bleaching agents include H₂O₂ and may be added in amounts less than 5%, preferably from 0.05 to 4%, calculated on the basis of the weight of the final composition.

Other bleaching components which might be comprised by the present invention include, peroxydiphosphate, urea, peroxide, metal peroxides such as calcium peroxide, sodium peroxide, stronthium peroxide, magnesium peroxide, hypochlorite salts such as sodium hypochlorite, and the salts of perborate, persilicate, perphosphate and percarbonate such as sodium perborate, potassium persilicate and sodium percarbonate. The peroxide compounds can be stabilized by addition of a triphenylmethane dye, a chelating agent or antioxidants such as butylated hydroxy anisole (BHA) or butylated hydroxy toluene (BHT).

### Solvent

A solvent is usually added to compositions of the invention in an amount sufficient for giving the compositions a flowable form in case the compositions is; e.g., a tooth paste, dental cream or gel, or to dissolve the other components of a compositions, in case of, e.g., a mouthwash or mouth rinse.

Suitable solvents include water, ethanol and water/ethanol mixtures, which may be present in an amount of from 0.1% to 70%.

### Anti-Microbial Agents

The present invention also includes water-soluble anti-microbial agents, such as chlorhexidine, triclosan, digluconate, hexetidine, alexidine, quaternary ammonium antibacterial compounds, and water-soluble sources of certain metal ions such as zinc, copper, silver and stannous (e.g., zinc, copper and stannous chloride, and silver nitrate) may also be included.

Sparingly soluble zinc salts such as zinc citrate, zinc C14-alkyl maleate, zinc benzoate, zinc caproate, zinc carbonate might also be included used in the compositions of the present invention to prolong the anti-microbial effectiveness of zinc ions due to the slow dissolution of these zinc salts in saliva.

Anti-microbial agents may be present in an amount of from 0% to 50% by weight, preferably from 0.01% to 40% by weight, most preferably from 0.1% to 30% by weight.

### Tartar-Controlling Agent

Compositions of the invention may comprise a tartar-controlling agent such as inorganic phosphorous tartar-controlling agents including any of the pyrophosphates such as disodium pyrophosphate, dipotassium pyrophosphate, tetrapotassium pyrophosphate, tetrasodium pyrophosphate, and mixtures thereof.

Organic phosphorous compounds that may serve as tartar-controlling agents include polyphosphonates such as disodium ethane-1-hydroxy-1,1-diphosphonate (EHDP), methanediphosphonic acid, and 2-phosphonobutane-1 2,4-tricarboxylic acid.

Tartar-controlling agents may be present in an amount of from 0% to 10% by weight, preferably from 0.1% to 5% by weight.

### Preservatives

Suitable preservatives include sodium benzoate, potassium sorbate, p-hydroxybenzoate esters, methyl paraben, ethyl paraben, propyl paraben, citric acid, calcium citrate, and mixtures thereof.

Preservatives may be present in an amount of from 0% to 40% by weight, preferably from 0.01% to 30% by weight.

### Fluoride Sources

Compositions of the invention may also comprise ingredients that can be used as fluoride source. Preferred soluble fluoride sources include sodium fluoride, potassium fluoride, stannous fluoride, indium fluoride, sodium monofluorophosphate, sodium hexafluorosilicate, zinc fluoride, lithium fluoride, aluminum fluoride, acidulated phosphate fluoride, ammonium bifluoride, titanium tetrafluoride, and amine fluoride.

Especially preferred are sodium fluoride and sodium monofluorophosphate.

Fluoride sources may be present in an amount of from 0% to 20% by weight, preferably from 0.01% to 15% by weight, most preferably from 0.1% to 10% by weight.

In a preferred embodiment, the at least one oral care ingredient is a fluoride source; preferably the fluoride source is selected from the group consisting of sodium fluoride, calcium fluoride, stannous fluoride, or sodium monofluorophosphate

### Coloring Agents

Coloring agents or pigments suitable for oral care compositions of the invention include non-toxic, water-insoluble inorganic pigments such as titanium dioxide and chromium oxide greens, ultramarine blues and pinks and ferric oxides as well as water insoluble dye lakes prepared by extending calcium or aluminum salts of FD&C dyes on alumina such as FD&C Green No. 1 lake, FD&C Blue No. 2 lake, FD&C Red No. 30 lake, FD&C Yellow No. 16 lake, and FD&C Yellow No. 10.

A preferred opacifier is titanium dioxide.

Coloring agents may be present in an amount of from 0% to 20% by weight, preferably from 0.01% to 15% by weight, most preferably from 0.1% to 10% by weight.

### Buffering Agents

The oral care compositions of present invention may also include buffering agents, i.e., pH-adjusting agents, such as alkali metal hydroxides, carbonates, sesquicarbonates, borates, silicates, phosphates, imidazole, and mixtures thereof.

Specific buffering agents include monosodium phosphate, trisodium phosphate, sodium hydroxide, potassium hydroxide, alkali metal carbonate salts, sodium carbonate, imidazole, pyrophosphate salts, sodium citrate, hydrochloric acid, sodium hydroxide, triethanolamine, triethylamine, lactic acid, malic acid, fumaric acid, tartaric acid, phosphoric acid and mixtures of these.

Buffering agents may be present in an amount of from 0% to 10% by weight, preferably from 0.01% to 5% by weight.

### Chewing Gum

When the oral composition according to the invention is a chewing gum, it can be any known type of chewing gum, such as chewing gum pieces optionally coated, as well as sticks or chewing gum provided with an arbitrary desired shape in response to the intended use. The chewing gum preparation can be of any quality including the bubble gum quality.

In a preferred embodiment, the present invention relates to enzymatic oral care compositions in the form of a chewing gum comprising a mutanase of the invention and at least one oral care ingredient, wherein the at least one oral care ingredient is selected from elastomer, softening agent, plasticizing agent, emulsifier, wax, coloring agent, sweetening agent, flavoring agent, bulking agent, and thickening agent.

### Gum Base Ingredients

Chewing gum is traditionally considered as being comprised of a water-insoluble or base portion and a water-soluble portion that contains flavoring agents, sweetening agents, and coloring agents. The gum base part of the gum is a masticatory substance which imparts the chew characteristics to the final product. It defines the release profile of flavors and the sweeteners and plays a significant role in the gum product. The flavors, sweeteners and colors can be thought of as providing the sensory appeal aspects of the chewing gum. No limitations as to the chewing gum bases used in a chewing gum preparation according to the invention exist. Conventional chewing gum bases available for instance from Dansk Tyggegummi Fabrik A/S, L.A. Dreyfus or Cafasa Gum SIA, are usually suitable, but specially made formulations can also be used. The formulation depends on the desired type of chewing gum or the desired type of structure. Suitable raw materials for gum bases include the substances according to the U.S. Chewing Gum Base Regulations—Code of Federal Regulations, Title 21, Section 172,615 and in accordance with other national and international lists (or positive lists) and include elastomers, resins, waxes, polyvinyl acetates, oils, fats, emulsifiers, fillers and antioxidants.

The gum base usually comprises from 15 to 90% by weight, preferably from 30 to 40% by weight, more preferably from 5 to 25% of the final product.

Elastomers provide the chew, springiness or bounce to the base and control bubble and flavor release in the final chewing gum. They may be any water-insoluble polymer known in the art. They include styrene butadiene copolymers (SBR) and non-SBR types, both natural and synthetic. Examples of natural elastomers include, without limitation, rubbers such as rubber latex (natural rubber) and guayule, and gums such as chicle, jelutong, balata, guttapercha, lechi capsi, sorva, crown gum, nispero, rosidinha, perillo, niger gutta, tunu, gutta kay, pendare, leche de vaca, chiquibul, crown gum, and the like, and mixtures thereof. Examples of synthetic elastomers include, without limitation, polyisobutylene, isobutylene-isoprene copolymers (butylrubber), polyethylene, polybutadiene, styrenebutadiene copolymers, polyisoprene, and the like, and mixtures thereof.

The amounts of elastomer (rubbers) employed in the gum base composition will vary greatly depending upon various factors such as the, type of gum base used (adhesive, or conventional, bubble or standard) the consistency of the gum base composition desired, and the other components used in the composition to make the final chewing gum product. In general, the elastomer is present in the gum base composition in an amount of from about 15% to about 60%, preferably from about 25% to about 30%, by weight based on the total weight of the gum base composition.

Elastomer solvents aid in softening or plasticizing the elastomer component. In doing so they provide a bulkiness to the chew.

Elastomer solvents include, but are not limited to, natural rosin esters and synthetic derivatives of, e.g., terpenes. Examples of elastomer solvents suitable for use herein include tall oil rosin ester; partially hydrogenated wood and gum rosin; the glycerol esters of wood and gum rosin, partially hydrogenated wood/gum rosin, partially dimerized wood and gum rosin, polymerized wood and gum rosin, and tall oil rosin; the deodorized glycerol ester of wood rosin; the pentaerythritol esters of wood and gum rosin; partially hydrogenated wood and gum rosin; the methyl ester of partially hydrogenated wood rosin; methyl, glycerol and pentaerythritol esters of rosins and modified rosins such as hydrogenated, dimerized and polymerized rosins; terpene resins such as polymers of alpha-pinene or beta-pinene, terpene hydrocarbon resins; polyterpene; and the like, and mixtures thereof. The elastomer solvent may be employed in the gum base composition in an amount of from about 2% to about 40%, and preferably from about 7% to about 15% by weight of the gum base composition.

Polyvinyl acetates provide stretch or elasticity to the gum base. They also affect chew bulkiness, softness and bubble, hydrophilic character and flavor release.

The amounts of the different molecular weight polyvinyl acetates present in the gum base composition should be effective to provide the finished chewing gum with the desired chew properties, such as integrity, softness, chew bulkiness, film-forming characteristic, hydrophilic character, and flavor release. The total amount of polyvinyl acetate used in the gum base composition is usually from about 45% to about 92% by weight based on the total gum base composition. The vinyl polymers may possess a molecular weight ranging from about 2000 Da up to about 95,000 Da.

Typically, the low molecular weight polyvinyl acetate has a weight average molecular weight of from about 2,000 Da to about 14,000 Da. The medium molecular weight polyvinyl acetate typically has a weight average molecular weight of from about 15,000 Da to 55,000 Da. The high molecular weight polyvinyl acetate typically has a weight average molecular weight of from 55,000 Da to about 95,000 Da but may range as high as 500,000 Da.

Waxes, fats, and oils plasticize the elastomer mixture and improve the elasticity of the gum base. Waxes can provide a soft or firm chew, affect the flavor release and provide bulkiness and smoothness to the gum base. Fats and oils provide a soft chew. The fats, oils and waxes may be use individually or in combination or the gum base may be a wax free gum base.

Waxes when used, may be of mineral, animal vegetable or synthetic origin. Non-limiting examples of mineral waxes include petroleum waxes such as paraffin and microcrystalline waxes, animal waxes include beeswax, vegetable waxes include carnauba, candellila, rice bran, esparto, flax and sugarcane, and synthetic waxes include those produced by the Fischer-Tropsch synthesis, and mixtures thereof.

Suitable oils and fats usable in gum compositions include hydrogenated or partially hydrogenated vegetable or animal fats, such as cottonseed oil, soybean oil, coconut oil, palm kernel oil, beef tallow, hydrogenated tallow, lard, cocoa butter, lanolin and the like; fatty acids such as palmitic, oleic, stearic, linoleic, lauric, myristic, caproic, caprylic, decanoic or esters and salts as sodium stearate and potassium stearate. These ingredients when used are generally present in amounts up to about 7% by weight of the gum composition, and preferably up to about 3.5% by weight of the gum composition.

Preferred as softeners are the hydrogenated vegetable oils and include soybean oil and cottonseed oil which may be employed alone or in combination. These softeners provide the gum base composition with good texture and soft chew characteristics. These softeners are generally employed in an amount from about 5% to about 14% by weight of the gum base composition.

Emulsifiers aid in dispersing the immiscible components of the gum base composition into a single stable system. They provide hydrophilic character to a gum base and aid in plasticizing the resins and polyvinyl acetates. They also affect the softness of the base and the, bubble character of the base. Typical emulsifiers include acetylated monoglyceride, glyceryl monostearate, lecithin, fatty acid monoglycerides, diglycerides, propylene glycol monostearate, lecithin, triacetin, glyceryl triacetate and the like, and mixtures thereof.

Preferred emulsifiers are glyceryl monostearate and acetylated monoglycerides. These serve as plasticizing agents. The emulsifiers may be employed in an amount of from about 2% to about 15% by weight of the gum base composition, and preferably from about 7% to about 11% by weight of the gum base composition.

The fats, oils, waxes, emulsifiers and certain sugar bulking agents are often grouped together and referred to as softening agents. Because of the low molecular weight of these ingredients, the softeners are able to penetrate the fundamental structure of the gum base making it plastic and less viscous. Useful plasticizers and softeners of the above include lanolin, palmitic acid, oleic acid, stearic acid, sodium stearate, potassium stearate, glyceryl triacetate, glyceryl lecithin, glyceryl monostearate, propylene glycol nonastearate, acetylated monoglyceride, glycerin, fully unsaturated vegetable oils such as nonhydrogenated cottonseed oil, hydrogenated vegetable oils, petroleum waxes, sorbitan monostearate, tallow, and the like, and mixtures thereof and also include high fructose corn syrup, corn syrup, sorbitol solution, hydrogenated starch hydrolysate, and the like, and mixtures thereof.

The amount of softener present should be an effective amount to provide a finished chewing gum with the desired chew bulkiness and softness. When used as softeners these materials are generally employed in the gum base composition in an amount of up to about 25%, and preferably in an amount of from about 1% to about 17%, by weight of the gum base composition.

The gum base may further contain a surfactant. Examples of suitable surfactants include polyoxyethylene (20) sorbitan monoleate, polyoxyethylene (20) sorbitan monolaurate, polyethylene (4) sorbitan monolaurate, polyoxyethylene (20) sorbitan monopalmitate, polyoxyethylene (20) sorbitan monostearate, polyoxyethylene, (4) sorbitan monostearate, polyoxyethylene (20) sorbitan tristearate, polyoxyethylene (5) sorbitan monooleate, polyoxyethylene (20) sorbitan trioleate, sorbitan monolaurate, and the like. The amount of surfactant present should be effective to provide the finished chewing gum with the desired softness. Typically, the surfactant is employed in the base in an amount of from about 0.5% to about 3.0% by weight based on the total weight of the gum base.

The gum base composition of this invention may also include effective amounts of fillers sometimes referred to as bulking agents. These materials add firmness and bulk and affect the texture and the flavor release of the chewing gum. Useful fillers include organic and inorganic compounds (mineral adjuvants) such as calcium carbonate, magnesium carbonate, ground limestone, magnesium silicate, calcium phosphate, cellulose polymers, clay, alumina, aluminum hydroxide, aluminum silicate, tale, tricalcium phosphate, dicalcium phosphate, and the like, and mixtures thereof. These fillers or adjuvants may be used in the gum base compositions in various amounts. The amount of the filler present should be effective to provide a finished chewing gum with the desired flavor release and integrity. Typically, the filler is employed in the gum base composition in an amount from about 1% to about 40%, and preferably from about 5% to about 20%, by weight of the gum base composition.

The gum base may also comprise an antioxidant to provide improved stability, lessen any oil-taste and provide longer shelf life. Typical non-limiting examples of antioxidants are butylated hydroxytoluene (BHT), butylated hydroxy anisole (BHA), propyl gallate. Mixtures thereof may also be used.

### Other Gum Ingredients

The remaining ingredients in chewing gum compositions are conventional and usually comprise from 10 to 85% by weight of the final product.

Examples thereof are sweetening agents, softeners, coloring agents, bulking agents, thickening agents, and flavoring agents of the type and in the amounts conventionally used for chewing gum.

Suitable flavoring agents those flavors known to the skilled artisan such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, wintergreen oil (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Other useful flavorings are artificial, natural and synthetic fruit flavors such as vanilla, and citrus oils including lemon, orange, lime, grapefruit, and fruit essences including apple, pear, peach, grape, strawberry, raspberry, cherry, plum, pineapple, apricot and so forth. These flavoring agents may be used in liquid or solid form and may be used individually or in admixtures. Commonly used flavors include mints such as peppermint, menthol, artificial vanilla, cinnamon derivatives, and various fruit flavors, whether employed individually or in admixture.

Other useful flavoring agents include aldehydes and esters such as cinnamyl acetate, cinnamaldehyde, citrate diethylacetal, dihydrocarvyl acetate, eugenyl formate, p-methyl anisole, and so forth may be used. Generally, any flavoring or food additive may be used.

Further examples of aldehyde flavorings include, but are not limited to, acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2-hexenal (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, mixtures thereof and the like.

The amount of flavoring agent employed herein is normally a matter of preference subject to such factors as the type of final chewing gum composition, the individual flavor, the gum employed, and the strength of flavor desired. Thus, the amount of flavoring may be varied in order to obtain the result desired in the final product and such variations are within the capabilities of those skilled in the art without the need for undue experimentation. In gum compositions, the flavoring agent is generally present in amounts from about 0.02% to about 5% by weight of the chewing gum composition.

The chewing gum compositions generally include bulking agents. These bulking agents (carders, extenders) may be water-soluble and include bulking agents selected from the group consisting of, but not limited to, monosaccharides, disaccharides, polysaccharides, sugar alcohols, and mixtures thereof; sorbitol, xylitol, maltitol, mannitol, isomalt (a racemic mixture of alpha-D-glucopyranosyl-1,6-mannitol and alpha-D-glucopyranosyl-1,6-sorbitol manufactured under the tradename Palatinit™ by Suddeutsche Zucker), glycerol, aspartame, Lycasin® glycerol, galactitol acesulphame K, saccharine and salts thereof, cyclamate and salts thereof, neohesperidine dihydrochalcone, glycyrrhizinic acid and salts thereof, thaumantine and sucralose as well as mixtures thereof or mixtures thereof with other suitable sweeteners, maltodextrins; hydrogenated starch hydrolysates; hydrogenated hexoses; hydrogenated disaccharides; minerals, such as calcium carbonate, talc, titanium dioxide, dicalcium phosphate, celluloses and the and the like, and mixtures thereof. Bulking agents may be used in amounts up to about 60%, and preferably in amounts from about 25% to about 60%, by weight of the chewing gum composition.

The chewing gum compositions may also include a high intensity sweetening agent (sweeteners). High intensity sweetening agents have a sweetness intensity substantially greater than that of sucrose. Examples of suitable intense sweeteners include:
a) water-soluble naturally-occurring intense sweeteners such as dihydrochalcones, monellin, steviosides, glycyrrhizin, dihydroflavenol, and L-aminodicarboxylic acid aminoalkonoic acid ester amides, such as those disclosed in in U.S. Pat. No. 4,619,834, and mixtures thereof;
b) water-soluble artificial sweeteners including the soluble saccharin salts such as sodium or calcium saccharin salts, cyclamate salts, the sodium, ammonium or calcium salts of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfam-K), the free acid form of saccharin, and the like, and mixtures thereof;
c) dipeptide based sweeteners including L-aspartic acid derived sweeteners such as 1-aspartyl-L-phenylalanine methyl ester (Aspartame) and materials described in U.S. Pat. No. 3,492,131, L-alpha-aspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2.5-dihydro-L-phenylalanine, L-aspartyl-L-(1-cyclohexen)-alanine, and the like, and mixtures thereof;
d) water-soluble intense, sweeteners derived from naturally-occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose®; examples of chlorodeoxysucrose and chlorodeoxygalactosucrose derivatives include but are not limited to: to 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-Ideoxy-beta-D-fructofuranoside, or 4,1′-dichloro-4,1′-dideoxygalactosucrose; 1′,6′-dichloro-1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galactopyranosyl-1,6-dichloro-1,6-dideoxy-beta-D-fluctofuranoside, or 4,6,1′,6′-tetrachloro-4,6,1′,6′-tetradeoxygalactosucrose; and 4,6,1′,6′-tetradeoxy-sucrose, and mixtures thereof; and
e) protein based intense sweeteners such as Thaumaoccous daniclii (Thaumatin I and II). The amount of sweetener employed in the chewing gum composition will vary with the sweetener selected for a particular chewing gum. Thus, for any given sweetener a sufficient amount of sweetener is used to provide the level of sweetness desired. The saccharide sweeteners and sugar alcohols described above are usually used in an amount of from about 1% to about 70% and preferably in an amount of from about 40% to about 50%, by weight based on the total weight of the chewing gum composition. The intense sweeteners described above are usually used in an amount of up to about 1%, preferably from about 0.05% to about 0.4%, by weight based on the total weight of the chewing gum composition.

The coloring agents useful in the present invention are used in amounts effective to produce the desired color. These coloring agents include pigments, which may be incorporated in amounts up to about 6%, by weight of the gum composition. A preferred pigment, titanium dioxide, may be incorporated in amounts up to about 2%, and preferably less than about 1%, by weight of the gum composition. The colorants may also include natural food colors and dyes suitable for food, drug and cosmetic applications. These colorants are known as F.D.& C. dyes and lakes. The materials acceptable for the foregoing uses are preferably water-soluble. Illustrative nonlimiting examples include the indigoid dye known as F.D.& C. Blue No. 2, which is the disodium salt of 5,5-indigotindisulfonic acid. Similarly, the dye known as F.D.& C. Green No. 1 comprises a triphenylmethane dye and is the monosodium salt of 4-[4-(N-ethyl-N-p-sulfoniumbenzylamino)diphenylmethylene]-[1-(N-ethyl-N-p-sulfoniumbenzyl)-delta-2,5-cyclo-hexadieneimine].

Examples of thickening agents include methyl cellulose, alginates, carrageenan, xanthan gum, gelatin, carob, tragacanth, and locust bean, emulsifiers, such as lecithin and glyceryl monostearate, acidulants such as malic acid, adipic acid, citric acid, tartaric acid, fumaric acid, and mixtures thereof.

The plasticizers, softening agents, emulsifiers, waxes, and antioxidants discussed above as being suitable for use in the gum base may also be used in the chewing gum composition.

### Active Gum Ingredients

Oral care compositions of the invention in the form of a chewing gum may also contain various active ingredients such as antimicrobial agents, Zn salts, fluorides, and urea.

Moreover, the oral composition according to the invention may, if desired, include any other active ingredients, such as anti-caries agents, anti-calculus agents, anti-plaque agents, anti-periodontal agents, anti-fungal agents, anti-smoking agents, anti-cold agents, agents against gingivitis, etc.

The antimicrobials used in the compositions can be any of a wide of cationic antimicrobial agents such as quaternary ammonium compounds (e.g., cetyl pyridinium chloride) and substituted guanidines such as chlorhexidine and the corresponding compound alexidine. Mixtures of cationic anti-microbials may also be used in the present invention.

Antimicrobial quaternary ammonium compounds include those in which one or two of the substituents on the quaternary nitrogen has a carbon chain length (typically alkyl group) of some 8 to 20, typically 10 to 18 carbon atoms while the remaining substituents (typically alkyl or benzyl group) have a lower number of carbon atoms, such as 1 to 7 carbon atoms, typically methyl or ethyl groups. Dodecyl trimethyl ammonium bromide, tetradecyl pyridinium chloride, tetradecyl ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis 2-ethylhexyl)-5-methyl hexa hydropyrimidine and benzethonium chloride are exemplary of typical quaternary ammonium antibacterial agents. Other compounds are the bis [4-(R-amino)-1-pyridinium]alkanes as disclosed in U.S. Pat. No. 4,206,215, Jun. 3, 1980 to Bailey incorporated herein by reference. The pyridinium compounds are the preferred quaternary ammonium compounds.

The cationic antimicrobial is generally used in the present compositions at a level of from about 0.02% to about 1%, preferably from about 0.3% to about 0.7% most preferably from about 0.3% to about 0.5%.

As easily soluble zinc salt it is in principle possible to use any physiologically acceptable, easily soluble zinc salt of an inorganic or organic acid, said salt being able to release zinc ions and being approved for the intended use, such as in foodstuffs, cosmetics or pharmaceutical products. Non-limiting examples are for instance zinc citrate, zinc sulphate, zinc lactate, zinc chloride, zinc acetate as well as mixtures thereof. Among these salts zinc acetate is preferred.

The zinc salt used must be easily soluble such that a release is ensured in the oral cavity of an amount of zinc ions efficient for the purpose aimed at within a suitable period of time.

Advantageously, the zinc salt is present in the oral composition in an amount of from 0.001 to 1.25% by weight. The amount used depends on the administration form and the intended use and is adapted such that an amount of zinc ions efficient for the intended use is released.

As taste-masking salt is used at least one salt selected among sodium chloride, ammonium chloride and physiologically acceptable alkali metal, alkaline earth metal and/or ammonium carbonates.

The alkali metal is in particular sodium or potassium, whereas the alkaline earth metal advantageously is calcium or magnesium. Particularly preferred taste-masking salts are sodium, potassium and magnesium carbonates, sodium chloride, ammonium chloride as well as mixtures thereof.

The taste-masking salt is advantageously used in the oral composition in an amount of from 0.05 to 6.25% by weight, more preferred from 0.25 to 3.50% by weight, such as from 0.50 to 2.50% by weight.

The amount used of taste-masking salt for masking the taste of zinc can in each case be determined by a person skilled in the art and depends on the particular zinc salt in question and the selected administration form.

Urea is used as an anticariogenic product for neutralizing the acid produced in dental plaque subsequent to eating or drinking. Beyond urea the composition also can contain pharmacologically acceptable substances capable of releasing urea under the conditions prevailing in the mouth. Examples thereof are: Salts and addition compounds between urea and inorganic compounds such as magnesium sulphate, calcium phosphate, sodium chloride, etc.

The urea content of the composition according to the invention varies between 0.05% by weight and 80% by weight, preferably between 0.2% by weight and 25% by weight.

The chewing gum compositions may be prepared using standard techniques and equipment known to those skilled in the art. The apparatus useful in accordance with the present invention comprises mixing and beating apparatus as well.

### Lozenges and Pastilles

Lozenges are flavored medicated dosage forms intended to be sucked and held in the mouth or pharynx. They may contain vitamins, antibiotics, antiseptics, local anesthetics, antihistamines, decongestants, corticosteroids, astringents, analgesics, aromatics, demulcents, or combinations of these ingredients. Lozenges may take various shapes, the most common being the flat, circular, octagonal, and biconvex forms. Another type, called bacilli, are in the form of short rods or cylinders. A soft variety of lozenge, called a pastille, consists of medicament in a gelatin or glycerogelatin base or in base of acacia, sucrose, and water (H. A. Lieberman, Pharmaceutical Dosage Forms: Tablets, Volume 1 (1980), Marcel Dekker, Inc., New York, N.Y.).

In a preferred embodiment, the present invention relates to enzymatic oral care compositions in the form of a lozenge or pastille comprising a mutanase of the invention and at least one oral care ingredient, wherein the at least one oral care ingredient is selected from lubricant, bulking agent, sweetening agent, and flavoring agent.

### Lubricants

The use of a lubricant in the manufacture of compressed lozenges is to facilitate the release of the lozenge from the die in which it is formed. The lubricant used in the present invention is a solid material which is not charged, and which will not interfere (e.g., complex) with the cationic antimicrobial. The material should preferably be water insoluble. One type of suitable material meeting these requirements is a non-toxic hydrocarbon fat or derivative. Examples include hydrogenated tallow and hydrogenated vegetable oil. Polyethylene glycols may also be used as a lubricant so long as they are solid materials which generally means having a molecular weight in the 4000 Da to 6000 Da range. These materials can also be used as a filler as noted below.

Mixtures of lubricants may also be used in the present invention. The lubricant is used at level of from about 0.1% to about 4.0% preferably from about 0.5% to about 2%.

### Lozenge Vehicle

The term “lozenge vehicle” is used herein to denote the material(s) which carries the active ingredients, enzymes and therapeutic agents, as well as the lubricant. These materials are also known as bulking agents or fillers. Since the vehicle is non-cariogenic, the vehicle should be free of sucrose and similar materials.

Acceptable filler materials include mannitol, sorbitol, xylitol, polyethylene glycol and noncariogenic dextrans. The fillers may be used alone or in combination.

Mannitol is a naturally occurring sugar alcohol and is available as a fine powder. It has a sweetness of only about 50% of that of sucrose. However, mannitol's negative heat of solution enables it to impart a pleasant, cooling sensation in the mouth as the lozenge dissolves. Sorbitol is a chemical isomer of mannitol and possesses a similar degree of sweetness. Its heat of solution, being negative, also provides for a pleasant, cooling sensation in the mouth. Sorbitol is available either as free flowing granules or as a crystalline powder. Polyethylene glycols (PEG's) can also be used in the present compositions. These materials are polymers of ethylene oxide with the generalized formula HOCH₂ (CH₂OCH₂)ₙCH₂OH. The use of PEG's alone is not favored but their use in combination with other fillers is acceptable. The molecular weights found most desirable are between 4000 Da and 6000 Da.

Fillers are generally used in the present invention at a level of from about 85% to about 99.8%, preferably from about 90% to about 98%, most preferably from about 94% to about 97%.

### Other Lozenge Components

Acceptable lozenges may be manufactured using just an active ingredient, the lubricant and the filler material as outlined above. However, in order to make the lozenges more acceptable from an aesthetic viewpoint, generally included are materials such as spray-dried or encapsulated flavors or liquid flavors adsorbed onto a suitable diluent. Spray-dried or encapsulated flavors are preferred. Suitable flavors include oil of peppermint, oil of wintergreen, oil of sassafras, oil of spearmint and oil of clove. Sweetening agents are also acceptable for use in the present compositions. Suitable agents include aspartame, acesulfame, saccharin, dextrose and levulose. Sweetening and flavoring agents are generally used in the compositions of this invention at levels of from about 0.1% to about 2%, preferably from about 0.25% to about 1.5%.

It is also acceptable to have a solid form of a water-soluble fluoride compound present in the present lozenges in an amount sufficient to give a fluoride concentration of from about 0.0025% to about 5.0% by weight, preferably from about 0.005% to about 2.0% by weight, to provide additionally anticaries effectiveness. Preferred fluorides are sodium fluoride, stannous fluoride, indium fluoride and sodium monofluorophosphate. The lozenges may also contain various active ingredients such as anti-microbial agents, Zn salts, fluorides, and urea (supra).

### Confectionaries and Candy

In a preferred embodiment, the present invention relates to enzymatic oral care compositions in the form of a confectionary or candy comprising a a mutanase of the invention and at least one oral care ingredient, wherein the at least one oral care ingredient is selected from coloring agent, sweetening agent, flavoring agent, and oil-modifying agent.

The preparation of confectionery formulations is historically well known and has changed little through the years. Confectionery items have been classified as either “hard” confectionery or “soft” confectionery. The volatile oil-modifying agent of the present invention can be incorporated by admixing the modifying agent into conventional hard and soft confections.

Hard confectionery may be processed and formulated by conventional means. In general, a hard confectionery has a base composed of a mixture of sugar and other carbohydrate bulking agents kept in an amorphous or glassy condition. This form is considered a solid syrup of sugars generally having from about 0.5% to about 1.5% moisture. Such materials normally contain up to about 92% corn syrup, up to about 55% sugar and from about 0.1% to about 5% water, by weight of the final composition. The syrup component is generally prepared from corn syrups high in fructose but may include other materials. Further ingredients such as flavorings, sweeteners, acidulants, colorants and so forth may also be added.

Such confectionery may be routinely prepared by conventional methods such as those involving fire cookers, vacuum cookers, and scraped-surface cookers also referred to as high speed atmospheric cookers.

Fire cookers involve the traditional method of making a candy base. In this method, the desired quantity of carbohydrate bulking agent is dissolved in water by heating the agent in a kettle until the bulking agent dissolves. Additional bulking agent may then be added, and cooking continued until a final temperature of 145 to 156° C. is achieved. The batch is then cooled and worked as a plastic-like mass to incorporate additives such as flavor, colorants and the like.

A high-speed atmospheric cooker uses a beat-exchanger surface, which involves spreading a film of candy on a heat exchange surface, the candy is heated to 165 to 170° C. in a few minutes. The candy is then rapidly cooled to 100 to 120° C. and worked as a plastic-like mass enabling incorporation of the additives, such as flavors, colorants and the like.

In vacuum cookers, the carbohydrate bulking agent is boiled to 125 to 132° C., vacuum is applied, and additional water is boiled off without extra heating. When cooking is complete, the mass is a semi-solid and has a plastic-like consistency. At this point, flavors, colorants, and other additives are admixed in the mass by routine mechanical mixing operations.

The optimum mixing required to uniformly mix the flavors, colorants and other additives during conventional manufacturing of hard confectionery is determined by the time needed to obtain a uniform distribution of the materials. Normally, mixing times of from 4 to 10 minutes have been found to be acceptable.

Once the candy mass has been properly tempered, it may be cut into workable portions or formed into desired shapes. A variety of forming techniques may be utilized depending upon the shape and size of the final product desired. A general discussion of the composition and preparation of hard confections may be found in H. A. Lieberman, Pharmaceutical Dosage Forms: Tablets, Volume 1 (1980), Marcel Dekker, Inc., New York, N.Y.

The apparatus useful in accordance with the present invention comprises cooking and mixing apparatus well known in the confectionery manufacturing arts, and election of the specific apparatus will be apparent to the artisan. In contrast, compressed tablet confections contain particular materials and are formed into structures under pressure.

These confections generally contain sugars in amounts up to about 95%, by weight of the composition, and typical tablet excipients such as binders and lubricants as well as flavoring agent, colorants and so forth. Similar to hard confectionery, soft confectionery may be utilized in this invention. The preparation of soft confections, such as nougat, involves conventional methods, such as the combination of two primary components, namely (1) a high boiling syrup such as corn syrup, hydrogenated starch hydrolysate or the like, and (2) a relatively light textured frappe, generally prepared from egg albumin, gelatin, vegetable proteins, such as soy derived compounds, sugarless milk derived compounds such as milk proteins, and mixtures thereof. The frappe is generally relatively light, and may, for example, range in density from about 0.5 to about 0.7 grams/cc.

The flavoring components of the confection are flavors having an associated bitter taste or other unpleasant after taste. These flavoring components may be chosen from natural and synthetic flavoring liquids such as volatile oils, synthetic flavor oils, flavoring aromatic and oils, liquids, oleoresins or extracts derived from plants, leaves, flowers, fruits, stew and combinations thereof. Non-limiting representative examples of volatile oils include spearmint oil, cinnamon oil, oil of wintergreen (methyl salicylate), peppermint oil, menthol, clove oil, bay oil, anise oil, eucalyptus oil, thyme oil, cedar leaf oil, oil of nutmeg, allspice oil, oil of sage, mace extract, oil of bitter almonds, and cassia oil. In addition, the confection may also contain artificial, natural or synthetic flavors including fruit flavors such as vanilla, and citrus oils including lemon, orange, grape, lime and grapefruit and fruit essences including apple, pear, peach, grape, strawberry, raspberry, cherry, plum, pineapple, apricot and so forth individual and mixed.

Other useful flavorings include aldehydes and esters such as benzaldehyde (cherry, almond), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyl-octanal (green fruit), and 2-dodecenal (citrus, mandarin), mixtures thereof and the like.

In the instance where sweeteners are utilized, the present invention contemplates the inclusion of those sweeteners well known in the art, including both natural and artificial sweeteners. The sweeteners may be chosen from the following non-limiting list: sugars such as sucrose, glucose (corn syrup), dextrose, invert sugar, fructose, and mixtures thereof, saccharin and its various salts such as the sodium or calcium salt; cyclamic acid and its various salts such as the sodium salt; the dipeptide sweeteners such as aspartame, dihydrachalcone compounds, glycyrrhizin; Stevia rebaudiana (Stevioside); chloro-derivatives of sucrose; dihydroflavinol; hydroxyguaiacol esters; L-amino dicarboxylic acid gem-diamines; L-aminodicarboxylic acid aminoalkenoic acid ester amides; and sugar alcohols such as sorbitol, sorbitol syrup, mannitol, xylitol, and the like. Also contemplated is the synthetic sweetener 3,6-dihydro-6-methyl-1,2,3-oxathiazin-4-one-2,2-dioxide, particularly the potassium (acesulfame-K), sodium and calcium salts thereof.

The confection may also include a colorant. The colorants may be selected from any of the numerous dyes suitable for food, drug and cosmetic applications, and known as FD&C dyes and the like. The materials acceptable for the foregoing spectrum of use are preferably watersoluble. Illustrative examples include indigoid dye, known as FD&C Blue No. 2, which is the disodium salt of 5,5′-indigotindisulfonic acid. Similarly, the dye known as FD&C Green No. 1 comprises a triphenylmethane dye and is the monosodium salts of 4-[4-N-ethyl-p-sulfobenzylami no)diphenylmethylane]-[1-(N-ethyl-N-p-sulfoniumbenzyl)-2-5-cyclohexadieneimine]. A full recitation of all FD&C and D&C dyes and their corresponding chemical structures may be found in the Kirk-Othmer Encyclopedia of Chemical Technology, in Volume 5.

The confectionary may also include a volatile oil-modifying agent such as capsicum oleoresin. An oil-modifying agent is present in an amount, which is undetected as a separate ingredient in the oral cavity, but nevertheless has the ability to modify sensory perception of the volatile oil.

The oil-modifying agent is present in an amount of from about 1 to about 150 ppm of the confection. The capsicum is available from Capsicum minimum, Capsicum frutescens, Capsicum annuum, and similar varieties. Commercially, the fruits of capsicum are referred to as chilies or as peppers. These fruits are known for their extreme potency of bite, pungency and characteristic odor.

With respect to confectionery compressed tablet formulations, such will contain a tablet granulation base and various additives such as sweeteners and flavors. The tablet granulation base employed will vary depending upon factors such as the type of base used, friability desired and other components used to make the final product. These confections generally contain sugars in amounts up to 95% by weight of the composition.

The confectionery compressed tablet may additionally include tablet excipients such as binders or lubricants, as well as flavoring agents, coloring agents, and volatile oils and volatile oil-modifying agents.

The variations that one may practice with regard to these confections are wide ranging and within the ability of those skilled in the art particularly with regard to the use of additional composition fillers, flavoring agents, the use of coloring agents, etc.

### External Oral Care Compositions

An external oral care formulation, e.g., denture cleaning solution, denture cleaning tablet, denture cleaning powder, and the like, may include ingredients and/or substances selected from the following categories:

| Type of ingredient | Examples | Amount (weight %) |
|---|---|---|
| Carrier liquid | Water, ethanol (18-27%), water/ | 0 to 95% |
| | ethanol mixture | |
| Disinfectant and bleaching | Sodium hypochlorite, Potassium | 0.001 to 95% |
| agents | Monopersulfate, Sodium Perborate | |
| Cleaning agents | Sodium bicarbonate, citric acid, | 0.001 to 95% |
| | sodium perborate, sodium | |
| | polyphosphate, potassium | |
| | monopersulfate | |
| Detergents and surfactants | Sodium lauryl sulfate (SLS) | 0.001 to 10% |
| Foaming agents | Polyethylene glycols (PEGs) | 0.0001 to 10% |
| Preservatives | Sodium benzoate, citric acid, | 0.001 to 75% |
| Flavoring agents | Peppermint, spearmint | 0.001 to 5% |

In a preferred embodiment the at least on oral care ingredient is selected from the group consisting of carrier liquids, disinfectant and bleaching agents, cleaning agents, detergents and surfactants, foaming agents, preservatives, and flavoring agents.

### Other Oral Care Compositions

The enzymatic oral compositions of the invention may also be included in filaments suitable for use in dental cleaning, e.g., filaments useful as dental floss. Preferably, the oral care composition is coated onto the exterior of the filament. Thus, in a preferred aspect, the present invention relates to a filament comprising an oral care composition comprising a mutanase of the invention and at least one oral care ingredient, wherein the filament is suitable for dental cleaning.

### Treatment of Oral Disease

The enzymatic oral care compositions of the invention are suitable for use in the treatment of oral disease, wherein removal of biofilm is desired. The compositions of the invention are particularly suitable for treating periodontal diseases and dental caries.

Periodontal disease, also known as gum disease, is a set of inflammatory conditions caused by bacterial infection and subsequent biofilm build-up on the teeth and the tissues surrounding the teeth. Periodontal disease may be divided in terms of severity into the following categories: gingivitis (including plaque-induced gingivitis), chronic periodontitis, aggressive periodontitis, periodontitis as a manifestation of systemic disease, necrotizing ulcerative gingivitis/periodontitis, abscesses of the periodontium, and combined periodontic-endodontic lesions. Periodontal disease may further be considered either localized or generalized depending on the extent of the affected area.

Dental caries, also known as tooth decay or cavities, is caused by organic acids, such as lactic acid, being released by certain biofilm-forming bacteria residing in the oral cavity, including Streptococcus mutans and some Lactobacillus species. Dental caries may be associated with further complications such as inflammation of the tissue around the teeth, tooth loss, and infection or abscess formation. Dental caries may be classified by location, etiology, rate of progression, and affected hard tissues, for instance according to the G.V. Black classification (class I, II, III, IV, V, and VI).

In one aspect, the present invention relates to an oral care composition comprising a mutanase selected from the group consisting of:
(a) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:3;
(b) a polypeptide having at least 90%, e.g., at least 90%, at least 91%, at least 92%, at least 93%, at least 94%, at least 95%, at least 96%, at least 97%, at least 98%, at least 99%, or 100%, sequence identity to SEQ ID NO:6;
(c) a polypeptide having at least 65%, e.g., at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:9;
(d) a polypeptide having at least 65%, e.g., at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO: 12;
(e) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO: 15;
(f) a polypeptide having at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO: 18;
(g) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:21;
(h) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:24;
(i) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:27;
(j) a polypeptide having at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:30; and
(k) a polypeptide having at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, sequence identity to SEQ ID NO:33;
for use as a medicament, wherein the polypeptide has endo-1,3-α-glucanase activity, and
wherein the oral care composition further comprises at least one oral care ingredient.

In an embodiment, the present invention relates to an oral care composition of the invention for use in the treatment of oral disease. In a preferred embodiment, the present invention relates to an oral care composition of the invention for use in the treatment of periodontal disease (e.g., gingivitis) and/or dental caries.

In one aspect, the present invention relates to use of an oral care composition of the invention for treatment or prophylactic treatment of a human subject.

In one aspect, the present invention relates to a method of treatment of a human subject, the method comprising administering an oral care composition of the invention to a human subject.

In one aspect, the present invention relates to a method for removing oral biofilm, the method comprising contacting the biofilm with an an oral care composition of the invention. In one embodiment, the oral care composition is an external oral care composition, and the biofilm is located on an object; preferably the object is a denture. In one embodiment, the object is located inside or outside the oral cavity.

The present invention is further described by the following examples that should not be construed as limiting the scope of the invention.

### EXAMPLES

### Example 1: Cloning, Expression, and Purification of Fungal Mutanases

Genes encoding four putative mutanases belonging to the glycosyl hydrolase family GH71 were cloned from the strains listed in Table 1 below. The strains were isolated from environmental samples collected in the countries indicated. The Trichoderma reesei strain was purchased from the Centraalbureau voor Schimmelcultures (CBS 383.78).

TABLE 1
Fungal mutanases

| Derived from | Source country | Mature polypeptide |
|---|---|---|
| Humicola insolens | Denmark | SEQ ID NO: 3 |
| Trichoderma reesei | Solomon Islands | SEQ ID NO: 6 |
| Talaromyces bacillisporus | Denmark | SEQ ID NO: 9 |
| Talaromyces apiculatus | USA | SEQ ID NO: 12 |

For all strains, chromosomal DNA was isolated and a whole genome sequence was determined using Illumina technology. Sequence data was assembled and annotated using standard methods as exemplified in WO 2019/228448, Example 11.

The polypeptides predicted from the annotated genomes were searched for similarity to the GH71 peptide domain as defined in the CAZy Database (www.cazy.org, Lombard V, et al. (2014) Nucleic Acids Res 42: D490-D495) and four polypeptides were found. Corresponding DNA sequences from all but Trichoderma reesei were cloned into the Aspergillus expression vector pMStr57 and expressed in Aspergillus oryzae as described in WO 2019/228448, Example 11. The mutanase from T. reesei was cloned and expressed in Aspergillus oryzae as described in WO 2020/200321, Example 5.

For larger-scale production of the recombinant enzymes, the Aspergillus transformants were cultured in 500 ml baffled flasks containing 150 ml of either DAP-4C-1 medium (WO 2012/103350) or YPG medium (WO 2005/066338). The cultures were shaken on a rotary table at 150 RPM for 4 days at 30° C., and the broth was subsequently separated from cellular material by passage through a 0.22 μm filtration unit. The pH of the filtered sample was adjusted to around pH 7.5 and 1.8 M ammonium sulfate was added. The sample was applied to a 5 ml HiTrap™ Phenyl (HS) column on an Äkta Explorer. Prior to loading, the column had been equilibrated in 5 column volumes (CV) of 50 mM HEPES+1.8 M AMS pH 7. In order to remove unbound material, the column was washed with 5 CV of 50 mM HEPES+1.8 M AMS pH 7. The target protein was eluted from the column into a 10 ml loop using 50 mM HEPES+20% isopropanol pH 7. From the loop, the sample was loaded onto a desalting column (HiPrep™ 26/10 Desalting), which had been equilibrated with 3 CV of 50 mM HEPES+100 mM NaCl PH 7.0. The target protein was eluted with 50 mM HEPES+100 mM NaCl pH 7.0 and relevant fractions were selected and pooled based on the chromatogram. The flow rate was 5 ml/min. Protein concentration in the final sample was estimated by measuring absorption at 280 nm.

### Example 2: Cloning, Expression, and Purification of Bacterial Mutanases

Chromosomal DNA was isolated from strains of Streptomyces yanii, Streptomyces galbus, Streptomyces glomeratus, Tetrasphaera sp-0185, and Kitasatospora phosalacinea, respectively, and subjected to full genome sequencing using Next Generation Sequencing technology. The DNA sequences were analyzed for polypeptides containing glycosyl hydrolase (GH) domains, as defined in the CAZy database (www.cazy.org, Lombard V, et al. 2014, Nucleic Acids Res 42: D490-D495), and five polypeptides containing a GH87 domain were identified (see Table 2).

Two other genes encoding polypeptides containing a GH87 domain were identified in strains of Streptomyces alni and Oribacterium sinus using public databases (see Table 2).

TABLE 2
Bacterial mutanases

| Strain | Source country | Mature polypeptide |
|---|---|---|
| Streptomyces yanii | USA | SEQ ID NO: 15 |
| Streptomyces alni | China | SEQ ID NO: 18 |
| Oribacterium sinus | Unknown | SEQ ID NO: 21 |
| Streptomyces galbus | USA | SEQ ID NO: 24 |
| Streptomyces glomeratus | USA | SEQ ID NO: 27 |
| Tetrasphaera sp-0185 | USA | SEQ ID NO: 30 |
| Kitasatospora phosalacinea | USA | SEQ ID NO: 33 |

The DNA encoding the mature polypeptide of the bacterial mutanases listed in Table 2 were ordered as synthetic genes from Twist Bioscience. The synthetic DNA fragments were directionally assembled to a Bacillus expression vector described in WO 2012/025577 by the standard Golden Gate cloning method using Bsal and T4 DNA ligase enzymes. Briefly, the DNA encoding the mature polypeptide of the genes was cloned in frame to a Bacillus clausii secretion signal (BcSP; with the following amino acid sequence: MKKPLGKIVASTALLISVAFSSSIASA (SEQ ID NO:34). BcSP replaced the native secretion signal in the gene. Downstream of the BcSP sequence, an affinity tag sequence was introduced to ease the purification process (His-tag; with the following amino acid sequence: HHHHHHPR (SEQ ID NO:35)). The genes that were expressed therefore comprised the BcSP sequence followed by the His-tag sequence followed by a mature polypeptide coding sequence.

The final expression plasmids were transformed into Bacillus subtilis expression hosts and integrated by homologous recombination into the host cell genomes upon transformation. The gene constructs were expressed under the control of a triple promoter system (as described in WO 1999/43835). The gene coding for chloramphenicol acetyltransferase was used as marker (as described in Diderichsen et al., 1993, Plasmid 30:312-315). Transformants were selected on LB media agar supplemented with six microgram of chloramphenicol per ml. One recombinant Bacillus subtilis clone per plasmid was selected and cultivated on a rotary shaking table in 500 ml baffled Erlenmeyer flasks each containing 100 ml yeast extract-based media.

After 3-5 days of cultivation at 30-37° C., the enzyme-containing supernatants were harvested by centrifugation and the enzymes was purified by His-tag purification using immobilized metal chromatography (IMAC) with Ni²⁺ as the metal ion on 5 mL HisTrap Excel columns (GE Healthcare Life Sciences). The purification took place at pH 7 and the bound protein was eluted with imidazole. The purity of the purified enzymes was checked by SDS-PAGE and the concentration of the enzyme determined by absorbance at 280 nm after buffer exchange in 50 mM HEPES, 100 mM NaCl PH 7.0

### Example 3: Mutanase Activity Assay

Mutanases (EC 3.2.1.59) produce reducing sugars upon hydrolysis of the mutan substrate. The release of reducing sugars may be detected and used to determine endo-1,3-α-glucanase activity.

The substrate was prepared by dissolving mutan (internally produced by Novozymes A/S according to the procedure described in Fuglsang et al., J. Biol. Chem., 2000, vol. 375, no. 3, pp. 2009-2018) in 0.5% of DMSO to a final concentration of 60 ppm (0.006%). Purified mature polypeptides were diluted to a stock concentration of 200 ppm (0.2 mg/mL). The reaction was initiated by incubating 60 ppm mutanase (0.06 mg/mL) together with 100 μL pre-heated mutan substate in 0.1 M sodium acetate buffer (pH 5.6) in a clear bottom 96-well microtiter plate (MTP; Thermo Scientific™, catalog number: 269620) for 10 minutes at 50° C. and 300 rpm. Samples without enzyme addition were used as substrate controls.

The mutan hydrolysis reaction was quenced by adding 80 μL alkaline reagent containing potassium sodium tartrate (50 g/L), PAHBAH (20 g/L), bismuth(III) acetate (5.52 g/L) and sodium hydroxide (0.5 M), and the quenched samples were incubated for an additional 20 minutes at 50° C. and 300 rpm. Mutanase activity was determined by measuring endpoint absorbance at 405 nm using a UV-Vis instrument (Synergy H1 Hybrid Multi-Mode Reader, Agilent Technologies) (see Table 3).

Appropriate substrate and enzyme blanks were included to correct for any free reducing group not emanating from the mutan. All chemicals and reagents were purchased by Sigma-Aldrich unless stated otherwise. The Trichoderma harzianum mutanase of SEQ ID NO:36 was included as comparator. As can be seen from Table 3, all mature polypeptides exhibited mutanase activity.

TABLE 3
Mutanase activity determination

| SEQ ID NO: | Absorbance (405 nm) |
|---|---|
| 3 | 0.74 |
| 6 | 1.31 |
| 9 | 1.17 |
| 12 | 1.12 |
| 15 | 0.90 |
| 18 | 0.90 |
| 21 | 0.67 |
| 24 | 1.08 |
| 27 | 1.07 |
| 30 | 0.93 |
| 33 | 0.86 |
| 36 | 1.29 |

### Example 4: Thermal Stability of Mutanases

Thermal stability measurements were performed using a capillary-based nano differential scanning fluorescence instrument (nanoDSF; Prometheus NT.Plex, NanoTemper Technologies GmbH, München, Germany). Standard nanoDSF grade capillary chips were used (Cat #: PR-AC002 from NanoTemper Technologies). The enzyme samples were loaded into the capillaries (each sample in triplicate) by capillary action. The emission intensities at 330 and 350 nm were determined automatically by the Prometheus NT.Plex. The fluorescence signals at 330 and 350 nm were monitored continuously as a function of temperature (heating rate used for thermal unfolding was 3.3° C. per minute from 20° C. to 95° C.). The data was analyzed using the PR. StabilityAnalysis_1.1.0.11077 software provided by the manufacturer. The analysis is model independent and takes the peak maximum of the first derivative which corresponds to the approximate thermal unfolding transition midpoint, defined as Tm.

Purified preparations of mutanases were diluted to a stock concentration of 2 mg/ml prior to a further 10 times dilution in citrate phosphate buffer (McIlvaine buffer, see below) and MilliQ water corresponding to a final protein concentration of 0.2 mg/ml. The Tm measurements for each enzyme was performed close to physiological pH range of the oral cavity using McIlvaine buffer at pH 5 and pH 6. 100 ml of McIlvaine buffer pH 5 was prepared by mixing 51.50 ml 0.2 M Na₂HPO₄+48.50 ml 0.1 M citric acid, whereas 100 ml McIlvaine buffer pH 6 prepared by mixing 63.15 ml 0.2 M Na₂HPO₄+36.85 ml 0.1 M citric acid.

TABLE 4
Thermal stability of mutanases at pH 5 and pH 6

Thermal stability (Tm, ° C.)

| SEQ ID NO: | pH 5 | pH 6 |
|---|---|---|
| 3 | 68.8 | 69.9 |
| 6 | 62.0 | 55.8 |
| 9 | 63.3 | 58.3 |
| 12 | 62.2 | 58.7 |
| 15 | 62.5 | 62.7 |
| 18 | 62.8 | 62.3 |
| 21 | 57.1 | 58.2 |
| 24 | 64.5 | 64.1 |
| 27 | — | 60.1 |
| 30 | 65.0 | 64.8 |
| 33 | 72.4 | 71.4 |
| 36 | 58.7 | 53.6 |

As shown in Table 4, the mutanases of the invention all have improved thermal stability at pH 5 and/or pH 6 compared to the T. harzianum mutanase of SEQ ID NO:36.

### Reproducibility of Thermal Stability Data

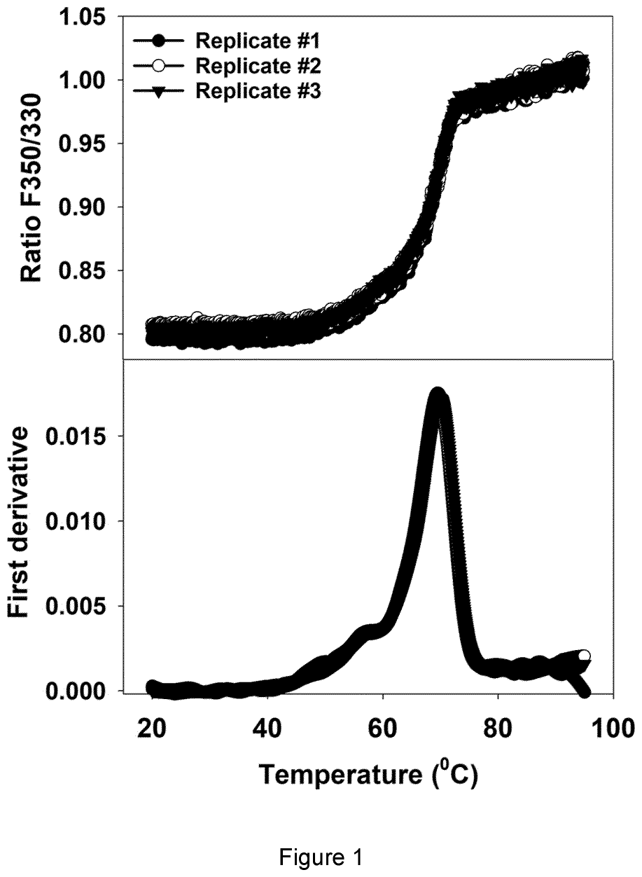

FIG. 1 shows an example of the thermal stability data generated using the nanoDSF instrument. Panel A is an example of the data obtained (the ratio of the fluorescence emission at 350 nm to 330 nm) in triplicate for SEQ ID NO:3 as a function of temperature. Panel B shows the first derivative of the raw data in Panel A. The peak maximum of the plot of the first derivative corresponds to the mid-point of the thermal unfolding transition, referred to as Tm. In this example, Tm corresponds to 69.6° C. at pH 6 and is highly reproducible within the three replicates. In all cases, the data showed a clear unfolding transition, and a clearly defined peak in the first derivative, and were highly reproducible.

### Example 5: Thermal Stability of Mutanases in the Presence of Oral Care Ingredients

### Preparation of Oral Care Formulations for Thermal Stability Measurements

The thermal stabilities or mid-point of the thermal unfolding transition (Tm) of the selected mutanases were measured in the presence oral care ingredients within the concentration range commonly used in oral care product formulations and selected oral care commercial products. The Tm parameter was used to evaluate the thermal stabilities as this is the temperature at which there are equal populations of folded and unfolded protein molecules and is a widely accepted parameter to use when evaluating thermal stability. Highly pure and biotechnology grade reagents were obtained from various suppliers and stock solutions were freshly prepared using MilliQ water. These formulation chemicals and their stock as well as the final concentrations used in the Tm measurement are listed in Table 5.

Selected purified mature mutanases were diluted to a stock concentration of 2 mg/ml prior to a further 10 times dilution in the oral care formulations consisting of individual oral care ingredients (concentrations specified in Tables 6 and 7), citrate phosphate buffer (McIlvaine buffer, see below) and MilliQ water corresponding to a final protein concentration of 0.2 mg/ml. Using robotics arm, all dilutions were made in a 384-well small volume deep well plate (Greiner Bio-One International, item number 784201) with a final volume of 70 μl and used for thermal stability measurements. The Tm measurements for each enzyme was performed close to physiological pH range of the oral cavity using McIlvaine buffer at pH 5 and pH 6. 100 ml of McIlvaine buffer pH 5 was prepared by mixing 51.50 ml 0.2 M Na₂HPO₄+48.50 ml 0.1 M citric acid, whereas 100 ml McIlvaine buffer pH 6 prepared by mixing 63. 15 ml 0.2 M Na₂HPO₄+36.85 ml 0.1 M citric acid.

TABLE 5
Formulation chemical and stock solutions

| Ingredient | Source | Product Number | Stock conc. (mM) |
|---|---|---|---|
| Sodium benzoate | Sigma | 18106 | 100 |
| EDTA | Sigma | E5134 | 10 |
| Ethanol | VWR Chemicals | 20.824.296 | 10000 |
| Glycerol | Sigma | G5516 | 7600 |
| Sodium phosphate | Sigma | 71643 | 50 |
| Sorbitol | Sigma | S6021 | 3000 |
| Potassium sorbate | Sigma | 85520 | 100 |
| Sodium fluoride | Sigma | 919 | 100 |
| Hydrogen peroxide | Sigma | H1009 | 1000 |
| Mannitol | Sigma | M9546 | 1000 |

### Thermal Stabilities in the Presence of Oral Care Ingredients

Tables 6 and 7 shows the average thermal stabilities of selected mutanases originating from triplicate measurements at pH 5 and 6, respectively, in the presence of oral care ingredients at the specified concentrations. From these data, it is evident that the tested formulation ingredients individually have no adverse effect on the thermal stability of the mutanases across the pH range tested compared to the control, i.e., when Tm is measured in the absence of formulation ingredients. Also, the mutanases of SEQ ID NO:3, SEQ ID NO:9, and SEQ ID NO:24 all exhibits improved stability compared to SEQ ID NO:36 under the majority of conditions tested.

TABLE 6
Thermal stability in the presence of oral care ingredients at pH 5

| | | Tm (° C.) at pH 5 | | | |
|---|---|---|---|---|---|
| | Conc. | SEQ ID | SEQ ID | SEQ ID | SEQ ID |
| Ingredient | (mM) | NO: 36 | NO: 3 | NO: 9 | NO: 24 |
| Control | 0 | 58.7 | 66.5 | 62.9 | 61.8 |
| EDTA | 1 | 54.3 | 66.4 | 63.0 | — |
| Ethanol | 1000 | 58.1 | 65.8 | 61.6 | 61.7 |
| Glycerol | 1000 | 55.3 | 67.8 | 63.8 | 62.9 |
| | 3250 | 63.6 | 71.1 | 66.1 | 64.3 |
| Hydrogen peroxide | 100 | 52.3 | 64.4 | 57.2 | 61.1 |
| | 500 | 44.7 | 58.7 | 54.3 | 59.2 |
| Potassium sorbate | 10 | 52.7 | 67.1 | 61.5 | 61.8 |
| | 35 | 54.7 | 65.6 | 59.6 | 61.6 |
| Mannitol | 250 | 59.9 | 67.2 | 63.6 | 62.6 |
| | 550 | 56.1 | 68.5 | 64.4 | 63.4 |
| Sodium benzoate | 10 | 53.3 | 66.0 | 62.3 | 61.6 |
| | 35 | 52.5 | 65.5 | 61.0 | 61.2 |
| Sodium fluoride | 20 | 53.6 | 66.4 | 62.9 | 61.9 |
| | 40 | 58.5 | 66.6 | 62.8 | 62.1 |
| Sodium phosphate | 5 | 50.4 | 69.0 | 58.3 | 61.6 |
| | 10 | 48.3 | 69.6 | 55.6 | 60.8 |
| Sorbitol | 550 | 56.0 | 67.8 | 64.1 | 63.3 |
| | 2200 | 64.5 | 74.6 | 70.5 | 68.7 |

TABLE 7
Thermal stability in the presence of oral care ingredients at pH 6

| | | Tm (° C.) at pH 6 | | | |
|---|---|---|---|---|---|
| | Conc. | SEQ ID | SEQ ID | SEQ ID | SEQ ID |
| Ingredient | (mM) | NO: 36 | NO: 3 | NO: 9 | NO: 24 |
| Control | 0 | 53.6 | 69.6 | 57.9 | 61.4 |
| EDTA | 1 | 59.0 | 69.5 | 57.9 | 59.9 |
| Ethanol | 1000 | 52.6 | 68.9 | 56.2 | 61.1 |
| Glycerol | 1000 | 60.3 | 71.1 | 58.6 | 62.1 |
| | 3250 | 58.7 | 75.1 | 61.2 | 63.2 |
| Hydrogen peroxide | 100 | 47.6 | 67.3 | 51.4 | 60.9 |
| | 500 | 49.1 | 59.9 | 49.6 | 59.3 |
| Potassium sorbate | 10 | 57.4 | 69.0 | 57.1 | 61.1 |
| | 35 | 50.5 | 66.8 | 55.9 | 60.6 |
| Mannitol | 250 | 54.8 | 70.2 | 58.5 | 62.0 |
| | 550 | 60.8 | 71.2 | 60.1 | 62.9 |
| Sodium benzoate | 10 | 58.2 | 69.2 | 57.2 | 61.1 |
| | 35 | 56.4 | 68.2 | 56.6 | 60.4 |
| Sodium fluoride | 20 | 59.0 | 69.5 | 57.4 | 61.4 |
| | 40 | 53.7 | 69.5 | 57.4 | 61.4 |
| Sodium phosphate | 5 | 54.6 | 69.7 | 54.8 | 60.4 |
| | 10 | 51.2 | 69.7 | 53.0 | 59.6 |
| Sorbitol | 550 | 60.9 | 71.2 | 59.2 | 62.9 |
| | 2200 | 68.2 | 77.7 | 65.9 | 68.0 |

### Example 6: Human Saliva Biofilm Prevention Assay

A biofilm prevention assay was carried out using human saliva according to the method described in WO 2020/099490 with some modifications. Briefly, biofilm was grown in 96-well plates either in the presence of McIlvaine buffer (pH 6; prepared by mixing 12.63 ml 0.2 M Na₂HPO₄ and 7.37 ml 0.1 M citric acid) as a negative control or in the presence of 60 ppm, 10 ppm, or 1 ppm mutanase dissolved in McIlvaine buffer, pH 6. The Trichoderma harzianum mutanase of SEQ ID NO:36 was included as comparator. Plates were incubated at 37° C. for 24 hours without shaking in a Thermo Scientific™ Rectangular AnaeroBox™ Container under microaerophilic conditions (ThermoScientific AnaeroGen 2.5L #AN0025A). Enzyme and control samples were measured as eight replicates.

After incubation, planktonic bacteria were removed by two gentle washes with 100 μl 0.9% NaCl and biofilms were stained with 0.095% crystal violet solution for 15 min at room temperature. Plates were rinsed twice with 100 μL 0.9% NaCl and adhered dye was dissolved with a solution of 96% ethanol and 0.1% acetic acid in water. Absorbance was measured at 600 nm with a microplate reader (SpectraMax M3, Molecular Devices).

For the data processing, the absorbance was taken to be proportional to the extent of remaining biofilm after enzyme or buffer treatment. The percentage of biofilm prevention was calculated as follows:

$$100 - ( ( A 600 nm enzyme treated sample ) / ( A 600 nm buffer control treated sample ) \times 100 ) ,$$

where A600 nm refers to the average of eight replicates of either enzyme-treated or control samples. Results are listed in Table 8.

TABLE 8
Human saliva biofilm prevention assay

| | % prevention at 60 | % prevention at 10 | % prevention at 1 ppm |
|---|---|---|---|
| Mutanase | ppm (mean ± SE) | ppm (mean ± SE) | (mean ± SE) |
| SEQ ID NO: 3 | 86.7 ± 1.2 | 80.0 ± 0.8 | 71.6 ± 1.4 |
| SEQ ID NO: 6 | 84.0 ± 0.4 | 81.9 ± 0.8 | 64.8 ± 2.2 |
| SEQ ID NO: 9 | 84.9 ± 0.4 | 81.0 ± 1.3 | 62.8 ± 1.7 |
| SEQ ID NO: 12 | 83.5 ± 0.6 | 79.9 ± 1.3 | 58.7 ± 2.0 |
| SEQ ID NO: 15 | 83.7 ± 0.9 | 84.2 ± 1.0 | 83.9 ± 0.6 |
| SEQ ID NO: 18 | 81.4 ± 1.0 | 83.7 ± 0.9 | 73.8 ± 1.8 |
| SEQ ID NO: 21 | 78.0 ± 0.7 | 84.0 ± 0.8 | 81.4 ± 1.0 |
| SEQ ID NO: 24 | 84.6 ± 0.8 | 84.4 ± 0.6 | 84.7 ± 0.7 |
| SEQ ID NO: 27 | 82.9 ± 0.9 | 83.8 ± 0.6 | 51.8 ± 2.4 |
| SEQ ID NO: 30 | 87.3 ± 0.5 | 81.5 ± 1.7 | 79.0 ± 1.8 |
| SEQ ID NO: 33 | 83.9 ± 0.7 | 84.7 ± 0.8 | 82.1 ± 0.8 |
| SEQ ID NO: 36 | 84.9 ± 0.5 | 84.2 ± 0.8 | 68.4 ± 1.8 |

### Example 7: PH Activity Profiles of Mutanases

The intraoral pH levels typically range between pH 6.5-7.8. However, saliva pH may vary due to dietary exposures, bacterial metabolism, salivary composition as well as time and location where saliva pH is measured. Subsequently, pH plays an important role in the solubility, stability, and activity of enzymes acting in the oral cavity such as the mutanases of the present invention. Thus, for oral care purposes, it is desirable to have mutanases with robust enzymatic activity in the dynamic pH environment of the oral cavity.

A substrate stock was prepared by dissolving 50 mg of mutan (internally produced by Novozymes A/S, vide supra) in 5 mL DMSO to a final stock concentration of 10 mg/mL. The substrate stock dissolved in DMSO was centrifuged at 12000 rpm for 2 minutes and the insoluble fraction was removed. Next, a working stock solution of mutan substrate (90 ug/mL) was prepared by further diluting the mutan stock solution (10 mg/mL) in a universal assay buffer (0.05 M glycine, HEPES, CAPS, succinate) with varying pH in the range of 4-10. Subsequently, 80 μL of 90 μg/mL working stock solution of mutan was equilibrated to 50° C. for 5 minutes in microtiter plate.

The enzymatic reaction for determining the relationship between enzymatic activity and pH was initiated by incubating 40 μL of prediluted mutanase enzyme (final assay concentration 20 g/mL) together with 80 μL equilibrated mutan substate at varying pH values ranging from 4 to 10 in a clear bottom 96-well microtiter plate (MTP; Thermo Scientific™) for 10 minutes at 50° C. and 300 rpm.

The mutan hydrolysis reaction was quenched by adding 80 μL alkaline reagent containing potassium sodium tartrate (50 g/liter), PAHBAH (20 g/liter), bismuth (III) acetate (5.52 g/liter) and sodium hydroxide (0.5 M). The quenched samples were incubated for an additional 20 minutes at 50° C. and 300 rpm. Mutanase activity was determined by measuring absorbance at 405 nm using a UV-Vis instrument (Synergy H1 Biotek Reader, Agilent Technologies). All samples, including blanks, were prepared in triplicates.

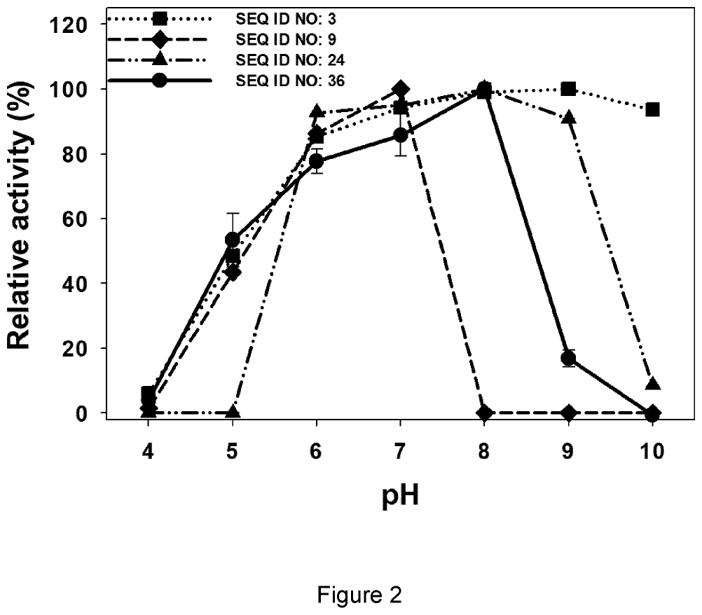

The relationship between endo-1,3-α-glucanase activity and pH is presented in FIG. 2. As can be seen, the mutanases of SEQ ID NO:3, SEQ ID NO:9, and SEQ ID NO:24 exhibit endo-1,3-α-glucanase activity at pH values that correspond to typical intraoral pH levels. Notably, SEQ ID NO: 3 exhibits endo-1,3-α-glucanase activity at pH 5-10, whereas SEQ ID NO:9 exhibits endo-1,3-α-glucanase activity at pH 5-7 and SEQ ID NO:24 exhibits endo-1,3-α-glucanase activity at pH 6-9.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention is further defined by the following numbered paragraphs:

1. An oral care composition comprising a mutanase selected from the group consisting of:
(a) a polypeptide having at least 60% sequence identity to SEQ ID NO:3;
(b) a polypeptide having at least 90% sequence identity to SEQ ID NO:6;
(c) a polypeptide having at least 65% sequence identity to SEQ ID NO:9;
(d) a polypeptide having at least 65% sequence identity to SEQ ID NO: 12;
(e) a polypeptide having at least 60% sequence identity to SEQ ID NO: 15;
(f) a polypeptide having at least 60% sequence identity to SEQ ID NO: 18;
(g) a polypeptide having at least 60% sequence identity to SEQ ID NO:21;
(h) a polypeptide having at least 60% sequence identity to SEQ ID NO:24;
(i) a polypeptide having at least 60% sequence identity to SEQ ID NO:27;
(j) a polypeptide having at least 60% sequence identity to SEQ ID NO:30; and
(k) a polypeptide having at least 60% sequence identity to SEQ ID NO:33;

wherein the polypeptide has endo-1,3-α-glucanase activity, and wherein the oral care composition further comprises at least one oral care ingredient.

2. The oral care composition according to paragraph 1, wherein the mutanase is selected from the group consisting of:
(a) a polypeptide comprising, consisting essentially of, or consisting of SEQ ID NO:3;
(b) a polypeptide comprising, consisting essentially of, or consisting of SEQ ID NO:6;
(c) a polypeptide comprising, consisting essentially of, or consisting of SEQ ID NO:9;
(d) a polypeptide comprising, consisting essentially of, or consisting of SEQ ID NO: 12;
(e) a polypeptide comprising, consisting essentially of, or consisting of SEQ ID NO: 15;
(f) a polypeptide comprising, consisting essentially of, or consisting of SEQ ID NO: 18;
(g) a polypeptide comprising, consisting essentially of, or consisting of SEQ ID NO:21;
(h) a polypeptide comprising, consisting essentially of, or consisting of SEQ ID NO:24;
(i) a polypeptide comprising, consisting essentially of, or consisting of SEQ ID NO:27;
(j) a polypeptide comprising, consisting essentially of, or consisting of SEQ ID NO:30; and
(k) a polypeptide comprising, consisting essentially of, or consisting of SEQ ID NO:33;
   3. The oral care composition according to any of paragraphs 1-2, wherein the mutanase has improved thermal stability compared to SEQ ID NO:36.
   4. The oral care composition according to any of paragraphs 1-3, wherein the mutanase has improved thermal stability compared to SEQ ID NO:36 when measured at pH 5 and/or pH 6 according to Example 4 herein; preferably at pH 5 or pH 6.
   5. The oral care composition according to any of paragraphs 1-4, wherein the mutanase has improved thermal stability compared to SEQ ID NO:36 in the presence of at least one oral care ingredient selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol.
   6. The oral care composition according to paragraph 5, wherein the mutanase has improved thermal stability compared to SEQ ID NO:36 when measured according to Example 5 herein at pH 5 and/or pH 6; preferably at pH 5 or pH 6.
   7. The oral care composition according to any of paragraphs 1-6, wherein the mutanase provides on par or improved, preferably improved, biofilm prevention compared to SEQ ID NO:36 when determined according to Example 6 herein; preferably the mutanase provides on par or improved biofilm prevention at about 1 ppm, about 10 ppm, and/or about 60 ppm.
   8. The oral care composition according to any of paragraphs 1-7 in the form of an internal oral care composition; preferably in the form of a toothpaste or toothpaste tablet, dental cream, mouthwash or mouthwash tablet, mouth rinse, lozenge, pastille, chewing gum, confectionary, or candy.
   9. The oral care composition according to any of paragraphs 1-7 in the form of an external oral care composition; preferably in the form of denture cleaning solution, denture cleaning tablet, or denture cleaning powder.
   10. The oral care composition according to any of paragraphs 1-9 for use as a medicament.
   11. The oral care composition according to any of paragraphs 1-9 for use in the treatment of oral disease; preferably for use in the treatment of periodontal disease (e.g., gingivitis) and/or dental caries.
   12. Use of an oral care composition according to any of paragraphs 1-9 for treatment or prophylactic treatment of a human subject.
   13. A method of treatment of a human subject, the method comprising administering an oral care composition according to any of paragraphs 1-9; preferably the oral care composition is administered to the oral cavity of the human subject.
   14. A method for preventing and/or removing oral biofilm, the method comprising contacting the oral biofilm with an oral care composition according to any of paragraphs 1-9.
   15. The method of paragraph 14, wherein the oral biofilm is located on an object, preferably a denture.
   16. The method according to paragraph 15, wherein the denture is located inside or outside the oral cavity.
   17. A kit of parts comprising:
(1) an oral care composition according to any of paragraphs 1-9; and
(2) instructions for use.
   18. A polypeptide having endo-1,3-α-glucanase activity selected from the group consisting of:
(a) a polypeptide having at least 60% sequence identity to SEQ ID NO:3;
(b) a polypeptide having at least 90% sequence identity to SEQ ID NO:6;
(c) a polypeptide having at least 65% sequence identity to SEQ ID NO:9;
(d) a polypeptide having at least 65% sequence identity to SEQ ID NO: 12;
(e) a polypeptide having at least 60% sequence identity to SEQ ID NO: 15;
(f) a polypeptide having at least 60% sequence identity to SEQ ID NO: 18;
(g) a polypeptide having at least 60% sequence identity to SEQ ID NO:21;
(h) a polypeptide having at least 60% sequence identity to SEQ ID NO:24;
(i) a polypeptide having at least 60% sequence identity to SEQ ID NO:27;
(j) a polypeptide having at least 60% sequence identity to SEQ ID NO:30; and
(k) a polypeptide having at least 60% sequence identity to SEQ ID NO:33.
   19. A polypeptide having endo-1,3-α-glucanase activity selected from the group consisting of:
(a) a polypeptide comprising, consisting essentially of, or consisting of SEQ ID NO:3;
(b) a polypeptide comprising, consisting essentially of, or consisting of SEQ ID NO:6;
(c) a polypeptide comprising, consisting essentially of, or consisting of SEQ ID NO:9;
(d) a polypeptide comprising, consisting essentially of, or consisting of SEQ ID NO: 12;
(e) a polypeptide comprising, consisting essentially of, or consisting of SEQ ID NO: 15;
(f) a polypeptide comprising, consisting essentially of, or consisting of SEQ ID NO: 18;
(g) a polypeptide comprising, consisting essentially of, or consisting of SEQ ID NO:21;
(h) a polypeptide comprising, consisting essentially of, or consisting of SEQ ID NO:24; (i) a polypeptide comprising, consisting essentially of, or consisting of SEQ ID NO:27;
(j) a polypeptide comprising, consisting essentially of, or consisting of SEQ ID NO:30; and
(k) a polypeptide comprising, consisting essentially of, or consisting of SEQ ID NO:33;
   20. The polypeptide according to any of paragraphs 18-19, which has improved thermal stability compared to SEQ ID NO:36.
   21. The polypeptide according to any of paragraphs 18-20, which has improved thermal stability compared to SEQ ID NO:36 when measured at pH 5 and/or pH 6 according to Example 4 herein; preferably at pH 5 or pH 6.
   22. The polypeptide according to any of paragraphs 18-21, which has improved thermal stability compared to SEQ ID NO:36 in the presence of at least one oral care ingredient selected from the group consisting of benzoate (e.g., sodium benzoate), EDTA, ethanol, glycerol, fluoride (e.g., sodium fluoride, sodium monofluorophosphate, calcium fluoride, or stannous fluoride), hydrogen peroxide, mannitol, phosphate (e.g., sodium phosphate or potassium phosphate), sorbate (e.g., potassium sorbate), and sorbitol.
   23. The polypeptide according to paragraph 22, which has improved thermal stability compared to SEQ ID NO:36 when measured according to Example 5 herein at pH 5 and/or pH 6; preferably at pH 5 or pH 6.
   24. The polypeptide according to any of paragraphs 18-23, which provides on par or improved, preferably improved, biofilm prevention compared to SEQ ID NO:36 when determined according to Example 6 herein; preferably the polypeptide provides on par or improved biofilm prevention at 1 ppm, 10 ppm, and/or 60 ppm.
   25. The polypeptide according to any of paragraphs 18-24, which is purified.
   26. The polypeptide according to any of paragraphs 18-24, which is isolated.
   27. A granule, which comprises:
(a) a core comprising the polypeptide of any one of paragraphs 18-26, and, optionally
(b) a coating consisting of one or more layer(s) surrounding the core.
   28. A granule, which comprises:
(a) a core, and
(b) a coating consisting of one or more layer(s) surrounding the core, wherein the coating comprises the polypeptide of any one of paragraphs 18-26.
   29. An oral composition comprising the polypeptide of any one of paragraphs 18-26 or the granule of paragraph 27 or 28 and at least on oral care ingredient.
   30. A liquid composition comprising the polypeptide of any one of paragraphs 18-26 and an enzyme stabilizer, e.g., a polyol such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).
   31. The liquid composition of paragraph 30, further comprising a filler or carrier material.
   32. The liquid composition of paragraph 30 or 31, further comprising a preservative.
   33. A composition comprising the polypeptide according to any of paragraphs 18-26, the granule of paragraph 27 or 28, or the liquid composition of any one of paragraphs 30-32.
   34. The composition of paragraph 33, which is a liquid composition, solid composition, solution, dispersion, paste, powder, granule, granulate, coated granulate, tablet, cake, crystal, crystal slurry, gel or pellet.
   35. A polynucleotide encoding the polypeptide of any one of paragraphs 18-24.
   36. The polynucleotide of paragraph 35, which comprises:
(a) SEQ ID NO:1 or nucleotides 1 to 87 and 142 to 1389 of SEQ ID NO: 1, in particular nucleotides 61 to 87 and 142 to 1389 of SEQ ID NO:1;
(b) SEQ ID NO:4 or nucleotides 1 to 150, 219 to 635, 705 to 1825, and 1883 to 2036 of SEQ ID NO:4, in particular nucleotides 73 to 150, 219 to 635, 705 to 1825, and 1883 to 2036 of SEQ ID NO:4;
(c) SEQ ID NO:7 or nucleotides 1 to 129, 196 to 727, 782 to 1007, 1063 to 1520, and 1582 to 2102 of SEQ ID NO:7, in particular nucleotides 61 to 129, 196 to 727, 782 to 1007, 1063 to 1520, and 1582 to 2102 of SEQ ID NO:7;
(d) SEQ ID NO: 10 or nucleotides 1 to 129, 190 to 721, 770 to 995, 1051 to 1511, and 1573 to 2111 of SEQ ID NO:10, in particular nucleotides 61 to 129, 196 to 727, 782 to 1007, 1063 to 1520, and 1582 to 2102 of SEQ ID NO:10;
(e) SEQ ID NO: 13 or nucleotides 124 to 3352 of SEQ ID NO:13;
(f) SEQ ID NO: 16 or nucleotides 76 to 2391 of SEQ ID NO: 16;
(g) SEQ ID NO: 19 or nucleotides 79 to 2226 of SEQ ID NO:19;
(h) SEQ ID NO:22 or nucleotides 79 to 2310 of SEQ ID NO:22.
(i) SEQ ID NO:25 or nucleotides 115 to 2184 of SEQ ID NO:25;
(j) SEQ ID NO:28 or nucleotides 133 to 2028 of SEQ ID NO:28; or
(k) SEQ ID NO:31 or nucleotides 115 to 2436 of SEQ ID NO:31.
   37. The polynucleotide of paragraph 35 or 36, which is purified.
   38. The polynucleotide of paragraph 35 or 36, which is isolated.
   39. A nucleic acid construct or expression vector comprising the polynucleotide of paragraph 35 or 36, operably linked to one or more control sequences that direct the production of the polypeptide in an expression host.
   40. A recombinant host cell comprising the nucleic acid construct or expression vector of paragraph 39.
   41. The recombinant host cell of paragraph 40, wherein the polypeptide is heterologous to the recombinant host cell.
   42. The recombinant host cell of paragraph 40 or 41, wherein at least one of the one or more control sequences is heterologous to the polynucleotide encoding the polypeptide.
   43. The recombinant host cell of any one of paragraphs 40-42, which comprises at least two copies, e.g., three, four, five, or more copies of the polynucleotide of any one of paragraphs 35-36.
   44. The recombinant host cell of any one of paragraphs 40-43, which is a yeast recombinant host cell, e.g., a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.
   45. The recombinant host cell of any one of paragraphs 40-43, which is a filamentous fungal recombinant host cell, e.g., an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell, in particular, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.
   46. The recombinant host cell of any one of paragraphs 40-43, which is a prokaryotic recombinant host cell, e.g., a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, or a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells.
   47. The recombinant host cell of any one of paragraphs 40-46, which is isolated.
   48. The recombinant host cell of any one of paragraphs 40-47, which is purified.
   49. A method of producing a polypeptide having endo-1,3-α-glucanase activity, comprising cultivating the recombinant host cell according to any of paragraphs 40-48 under conditions conducive for production of the polypeptide.
   50. The method of paragraph 49, further comprising recovering the polypeptide.
   51. A method of producing the polypeptide of any one of paragraphs 18-24, comprising cultivating a cell, which in its wild-type form produces the polypeptide, under conditions conducive for production of the polypeptide.
   52. The method of paragraph 51, further comprising recovering the polypeptide.
   53. A whole broth formulation or cell culture composition comprising the polypeptide of any one of paragraphs 18-24.

1-15. (canceled)

16. An oral care composition comprising a mutanase selected from the group consisting of:
(a) a polypeptide having at least 60% sequence identity to SEQ ID NO: 3;
(b) a polypeptide having at least 90% sequence identity to SEQ ID NO: 6;
(c) a polypeptide having at least 65% sequence identity to SEQ ID NO: 9;
(d) a polypeptide having at least 65% sequence identity to SEQ ID NO: 12;
(e) a polypeptide having at least 60% sequence identity to SEQ ID NO: 15;
(f) a polypeptide having at least 60% sequence identity to SEQ ID NO: 18;
(g) a polypeptide having at least 60% sequence identity to SEQ ID NO: 21;
(h) a polypeptide having at least 60% sequence identity to SEQ ID NO: 24;
(i) a polypeptide having at least 60% sequence identity to SEQ ID NO: 27;
(j) a polypeptide having at least 60% sequence identity to SEQ ID NO: 30; and
(k) a polypeptide having at least 60% sequence identity to SEQ ID NO: 33;
wherein the polypeptide has endo-1,3-α-glucanase activity, and wherein the oral care composition further comprises at least one oral care ingredient.

17. The oral care composition according to claim 16, wherein the mutanase is selected from the group consisting of:
(a) a polypeptide comprising, consisting essentially of, or consisting of SEQ ID NO: 3;
(b) a polypeptide comprising, consisting essentially of, or consisting of SEQ ID NO: 6;
(c) a polypeptide comprising, consisting essentially of, or consisting of SEQ ID NO: 9;
(d) a polypeptide comprising, consisting essentially of, or consisting of SEQ ID NO: 12;
(e) a polypeptide comprising, consisting essentially of, or consisting of SEQ ID NO: 15;
(f) a polypeptide comprising, consisting essentially of, or consisting of SEQ ID NO: 18;
(g) a polypeptide comprising, consisting essentially of, or consisting of SEQ ID NO: 21;
(h) a polypeptide comprising, consisting essentially of, or consisting of SEQ ID NO: 24;
(i) a polypeptide comprising, consisting essentially of, or consisting of SEQ ID NO: 27;
(j) a polypeptide comprising, consisting essentially of, or consisting of SEQ ID NO: 30; and
(k) a polypeptide comprising, consisting essentially of, or consisting of SEQ ID NO: 33.

18. The oral care composition according to claim 16, wherein the mutanase has improved thermal stability compared to SEQ ID NO: 36 when measured at pH 5 and/or pH 6 according to Example 4 herein.

19. The oral care composition according to claim 16, wherein the mutanase has improved thermal stability compared to SEQ ID NO:36 in the presence of at least one oral care ingredient selected from the group consisting of benzoate, EDTA, ethanol, glycerol, fluoride, hydrogen peroxide, mannitol, phosphate, sorbate, and sorbitol when measured according to Example 5 herein at pH 5 and/or pH 6.

20. The oral care composition according to claim 16, wherein the mutanase provides on improved biofilm prevention compared to SEQ ID NO: 36 when determined according to Example 6 herein.

21. A method for prevention and/or removing oral biofilm, the method comprising contacting the oral biofilm with an oral care composition according to claim 16.

22. A method for the treatment of a periodontal disease of a human subject, the method comprising administering an oral care composition according to claim 16 to the human subject.

23. The method of claim 22 wherein the periodontal disease is gingivitis.

24. A kit of parts comprising:
(1) an oral care composition according to claim 16; and
(2) instructions for use.

25. A polypeptide having endo-1,3-α-glucanase activity selected from the group consisting of:
a. a polypeptide having at least 60% sequence identity to SEQ ID NO: 3;
b. a polypeptide having at least 90% sequence identity to SEQ ID NO: 6;
c. a polypeptide having at least 65% sequence identity to SEQ ID NO: 9;
d. a polypeptide having at least 65% sequence identity to SEQ ID NO: 12;
e. a polypeptide having at least 60% sequence identity to SEQ ID NO: 15;
f. a polypeptide having at least 60% sequence identity to SEQ ID NO: 18;
g. a polypeptide having at least 60% sequence identity to SEQ ID NO: 21;
h. a polypeptide having at least 60% sequence identity to SEQ ID NO: 24;
i. a polypeptide having at least 60% sequence identity to SEQ ID NO: 27;
j. a polypeptide having at least 60% sequence identity to SEQ ID NO: 30; and
k. a polypeptide having at least 60% sequence identity to SEQ ID NO: 33.

26. The polypeptide according to claim 25, which has improved thermal stability compared to SEQ ID NO: 36 when measured at pH 5 and/or pH 6 according to Example 4 herein.

27. A polynucleotide encoding the polypeptide of claim 25.

28. A nucleic acid construct or expression vector comprising the polynucleotide of claim 25 operably linked to one or more control sequences that direct the production of the polypeptide in an expression host.

29. A recombinant host cell comprising the nucleic acid construct or expression vector of claim 28.

30. A method of producing a polypeptide having endo-1,3-α-glucanase activity, the method comprising cultivating the recombinant host cell of claim 29 under conditions conducive for production of the polypeptide.


SEQUENCE LISTING (36 sequences)

SEQ ID NO: 1 - DNA, 1389 bp, Humicola insolens
  source 1..1389: mol_type = genomic DNA
atgaagctcctatctacggccttgacggctggggccctggcagtcatgcccgtccaggcgctgaaagaggtgtttgcccatgttattgtgagttcctgactgttcttcgtcaattgaagcccctactgacaataaagtcagactggcaacatgcccgagatgcaggtctcggagtgggaagccgacatgcgtctcgccaaggaggcccacatcgatggcttcgtgctcaatattgcctcacaggacgcctcgaacaacaactccatcgcccgcgccttcgaggctgccgagagggtgggcaacttcaagctcttcttctcattcgactacgaggctcagggcccctggcccgccgacagagttgccaacctcctggccgagtacatctggcgtgcgccctactttaagcaccagggcaaccgccctctcgtgtcgacatttgagggacccaacaacgccaacgactggaacaccatcaagcagttggtgggcaacatcttcttcatccctgactggtcgtctcgggcgccaaacgaggccatcgggctcggcggcggcgtggcggacgggctcttcaactgggacgcatggccggagggtaagaatgacaagacgactgcgagcgaccaagctttccgcgcagccctcggcggcaagccgtacatgatgcccgtctccccatggttcttcaccaacctgcccgggtggaacaagaattggctgtggaagggcgacggtctgtgggatctgcgctggcggcaggtggcagaggtgaagcctgagtacgtccagatcttgacgtggaacgactttggtgagtctcactacattggcccagtgcacgagaaacaactggcgctgttcaactacggccaggcaccaatcaattatgcacggaacatgcctcatgatgcctggcgcaagttcctgcctttctgcatcgacgtgtacaagaacgacggcagggtacccgccaacgtcggccaagagggcgtcgtggcatactaccgcatcgccccggcgcgggcttgccccagcgggggaacgacgggcaacaacccgaaccatggccagaccgtgttgcctcccgaagaggtcctatcggacaacgtcttcttcgcggcgctgctgaacacgaacaggggcgtggaggtgacggtgtctattgggggtctggaacaaaaggcaagcgtgtttacgctggtcccggccgcgggtgcaggaacgccgggtgtgtacacgggtagcgtgcccttcggggatcgcacaggtgatgtggtggttacggtgaagagggacggcaacacgatcgctacggcgaggggaagcaggccgatctcgaggacgtgtgagaataatgtccagaactggaatgcggtggttgcttaa

SEQ ID NO: 2 - protein, 444 aa, Humicola insolens
MKLLSTALTAGALAVMPVQALKEVFAHVITGNMPEMQVSEWEADMRLAKEAHIDGFVLNIASQDASNNNSIARAFEAAERVGNFKLFFSFDYEAQGPWPADRVANLLAEYIWRAPYFKHQGNRPLVSTFEGPNNANDWNTIKQLVGNIFFIPDWSSRAPNEAIGLGGGVADGLFNWDAWPEGKNDKTTASDQAFRAALGGKPYMMPVSPWFFTNLPGWNKNWLWKGDGLWDLRWRQVAEVKPEYVQILTWNDFGESHYIGPVHEKQLALFNYGQAPINYARNMPHDAWRKFLPFCIDVYKNDGRVPANVGQEGVVAYYRIAPARACPSGGTTGNNPNHGQTVLPPEEVLSDNVFFAALLNTNRGVEVTVSIGGLEQKASVFTLVPAAGAGTPGVYTGSVPFGDRTGDVVVTVKRDGNTIATARGSRPISRTCENNVQNWNAVVA

SEQ ID NO: 3 - protein, 424 aa, Humicola insolens
LKEVFAHVITGNMPEMQVSEWEADMRLAKEAHIDGFVLNIASQDASNNNSIARAFEAAERVGNFKLFFSFDYEAQGPWPADRVANLLAEYIWRAPYFKHQGNRPLVSTFEGPNNANDWNTIKQLVGNIFFIPDWSSRAPNEAIGLGGGVADGLFNWDAWPEGKNDKTTASDQAFRAALGGKPYMMPVSPWFFTNLPGWNKNWLWKGDGLWDLRWRQVAEVKPEYVQILTWNDFGESHYIGPVHEKQLALFNYGQAPINYARNMPHDAWRKFLPFCIDVYKNDGRVPANVGQEGVVAYYRIAPARACPSGGTTGNNPNHGQTVLPPEEVLSDNVFFAALLNTNRGVEVTVSIGGLEQKASVFTLVPAAGAGTPGVYTGSVPFGDRTGDVVVTVKRDGNTIATARGSRPISRTCENNVQNWNAVVA

SEQ ID NO: 4 - DNA, 2063 bp, Trichoderma reesei
  source 1..2063: mol_type = genomic DNA
atgtttggtcttgtccgccgactcggggtcggcgcccttgtcgccgcagccctttcctccctcgctgccgccgcgccagccaacgtcgccatccgctccctcgaggaacgggctagcagcgcagatagacttgtgttttgccactttatggtgcgtttgtccgccccaagagcattgaaaattcagtaatcactgacacgccttactcgtgatgctagattgggatatgtggtgatcgcacctccagtaccgattatgatgatgatatgcagcgagccaaggccgcgggcattgacgcctttgcccttaacattggtgtcgacggatacacggaccagcagctcaactttgcctacgacgccgctgatcgcgccgggatgaaggtgttcatctcctttgacttcaactggtggagccccggcaacgcggcaggcgtcggccagaagattgcccaatatgcgtcgcggcccgcacagctctacgtcgacaaccgtccctttgcatcgtcgtttgccggtgacggccttgacgtgaatacgctgcggaatgcggccggcagcaacgtgtactttgtgcccaacttccaccccgggcagtcgtcgccgtccaccatcgacggggctctgaactggatggtacgtctgggcgttgtggctcgaggataaagcaaagaccaagtactcatgcgctgacacgctccacaggcctgggacaacgacggcaacaacaaggcccccaagcccggccaaaacgtcacagtcgccgacggcgacaactcctaccgcagctggctcgccggcaagccctacctcgcccccgtctcgccctggttcttcacccacttcggcccagaggtatcgtacagtaagaactgggtcttccctggcggctccctgtggtacgaccgctggcaggacgtgctgcgccagggcttcgagatggtcgaaatcgtcacctggaacgattacggtgagagccactacacggggcccctggaaagtcgacactatgacgacggaaactcgaaatggaccaacgacatgccgcacgacggcttcctggacctggcgaagccattcattgccgcgtacaagaaccgcgacacggacgtggcgccctacatccagaatgagcagctgatctactggtatcggcggaatctcaaggggctggactgcgacgcgaccgacacgacgtcgaaccgcccggcgaacaacggcagcggcaactacttcatgggtcggcccgacgggtggcagacgatggacgacacggtgtatgtggtggcgctgctcaagagcgcgggcacggtgacggtgacgtcgggcggcgccacgcagacgttccagggcaccgccggcgccaacctgttcgaggtcccagccaaccttgggcagcagaagtttgccctgtcccgcaacgggcagaccgtcttcagcagcacgtcgctgatggatatcaccaatgtgtgcccgtgcggcatctacaacttcaacccgtatgtcgggactgtgcccgctggctttgacgacccgctcgggcccgatggccttgcttctttgacaatcggactgcacgtcacgacttgtcaggccaagccgtcgctggggaccaacccgcccatcacttccggccccggctcctcggtgcccgtttccactccgcccggttccacgacccgcttctcgtcaacgccggtttcatctcgctccagctcgtccacgccgccggttagcacgccgccgcctggccaagtctgtgtcgccggtacggtggctgacggccagtccggcaactatattggcctctgcaacttcagctgcaagtaagttaccccatgtctcatgacgatgtatctccgacaccagctaacgtttgccagcttcgggtactgtccccccggaccctgcaagtgcactgcctacggcgctccgatcaacccaccagcaacgaatgggcgaaatgggtgccccttgcctggagaagacgatagttatctgggcctctgcagcttcagctgcaaccacaattactgtccgccaacagcatgccagtactgctga

SEQ ID NO: 5 - protein, 622 aa, Trichoderma reesei
MFGLVRRLGVGALVAAALSSLAAAAPANVAIRSLEERASSADRLVFCHFMIGICGDRTSSTDYDDDMQRAKAAGIDAFALNIGVDGYTDQQLNFAYDAADRAGMKVFISFDFNWWSPGNAAGVGQKIAQYASRPAQLYVDNRPFASSFAGDGLDVNTLRNAAGSNVYFVPNFHPGQSSPSTIDGALNWMAWDNDGNNKAPKPGQNVTVADGDNSYRSWLAGKPYLAPVSPWFFTHFGPEVSYSKNWVFPGGSLWYDRWQDVLRQGFEMVEIVTWNDYGESHYTGPLESRHYDDGNSKWTNDMPHDGFLDLAKPFIAAYKNRDTDVAPYIQNEQLIYWYRRNLKGLDCDATDTTSNRPANNGSGNYFMGRPDGWQTMDDTVYVVALLKSAGTVTVTSGGATQTFQGTAGANLFEVPANLGQQKFALSRNGQTVFSSTSLMDITNVCPCGIYNFNPYVGTVPAGFDDPLGPDGLASLTIGLHVTTCQAKPSLGTNPPITSGPGSSVPVSTPPGSTTRFSSTPVSSRSSSSTPPVSTPPPGQVCVAGTVADGQSGNYIGLCNFSCNFGYCPPGPCKCTAYGAPINPPATNGRNGCPLPGEDDSYLGLCSFSCNHNYCPPTACQYC

SEQ ID NO: 6 - protein, 598 aa, Trichoderma reesei
APANVAIRSLEERASSADRLVFCHFMIGICGDRTSSTDYDDDMQRAKAAGIDAFALNIGVDGYTDQQLNFAYDAADRAGMKVFISFDFNWWSPGNAAGVGQKIAQYASRPAQLYVDNRPFASSFAGDGLDVNTLRNAAGSNVYFVPNFHPGQSSPSTIDGALNWMAWDNDGNNKAPKPGQNVTVADGDNSYRSWLAGKPYLAPVSPWFFTHFGPEVSYSKNWVFPGGSLWYDRWQDVLRQGFEMVEIVTWNDYGESHYTGPLESRHYDDGNSKWTNDMPHDGFLDLAKPFIAAYKNRDTDVAPYIQNEQLIYWYRRNLKGLDCDATDTTSNRPANNGSGNYFMGRPDGWQTMDDTVYVVALLKSAGTVTVTSGGATQTFQGTAGANLFEVPANLGQQKFALSRNGQTVFSSTSLMDITNVCPCGIYNFNPYVGTVPAGFDDPLGPDGLASLTIGLHVTTCQAKPSLGTNPPITSGPGSSVPVSTPPGSTTRFSSTPVSSRSSSSTPPVSTPPPGQVCVAGTVADGQSGNYIGLCNFSCNFGYCPPGPCKCTAYGAPINPPATNGRNGCPLPGEDDSYLGLCSFSCNHNYCPPTACQYC

SEQ ID NO: 7 - DNA, 2102 bp, Talaromyces bacillisporus
  source 1..2102: mol_type = genomic DNA
atgatgatgaaaaatgtcgtttttgcgatgctatccgcctttgccgggctggtccaagggctcccaggatcagacagatcattaactgcgaggcagtcaactgacaggctcgtctttgctcattttatggtatgtcctgtagcacctagcctttaatggttgccgaaggagggaactttttctaagagctcatagataggaattacaagtgatcgtcacagtgcgtcagactttgatgcagacatgcaacgcgctaaagcatatggaattgatgcctttgctctgaatattggtgtcgacccatacaccgatgaagaactgggatacgcctatgactctgcagcaaacaatggaatgaaagtttttatctcatttgactttaattggtggaataccggccaggctaccgcaataggacaaaagattgctcagtacgcgagcaagccagcccagctcatggttgataacaaggttttcgtttcatcctttgctggcgatggggttgatgtagcgggtatccgatccgctgcgggtatggatttattcttcgcaccgaactttcatccttcatatggtacagatttgtcatctgttgacggattgctgaactggatggcgtggcccaacaatggcaacaacaaggcaccgactgcaggggccaatgtcaccgttcaagacggcgatgagcaatacatcaatgctcttggtggcaaggcctatattgctcgtaagacattgatgatttgaattatatggacacctaagctaacatattgtcaagctgcctcaccttggttttcaactcatttcggacccgaggtttcatacagcaagaactgggttttcccatctgacctgctctggtacaaccgttggcaagaccttttgaccctaggtccccgttttattgagatcgttacatggaatgattacggagagtctcactatatcgggccattggattctcctcacaccgacgatggggcttctaaatgggtcaatgacatgtatggttttagttaatctgtgtcttactttatgttaatactaacacaactccaggcctcatgacggttggctcgatatggcgaagccctttattgccgcattcaaagatggcgctaccagtatcgatagctacatcacagatgatcagctcatttactggtataggcctaccccacgcggcgttgactgcgatgctacagatacctgcatggtttcagcccctaatgcctcgggaaactattttattggacgtcccaatggatgggagacaatggaagactctgtctttgtagtttcactcttcaaatcggccgccactatcaccgtgaattcaggaggaaatgtacagacctttaatgcaccctctggagccagctcctttgaagtcccaatgggtgtcggtgcacagacctttagcgttaccaggaacggacagactgtttttgaggggactagtttaaagcagatcataaacggatgcgtgtgtgggttgtataattttaatgcttatggtcggttgtttctctcctgacatttacttcgaatatcataatgctgattgtgcacatgcagttggaagtattccagccggcttcgctgaccctctccaaccggatggactcgcatctttaactcaaggcctgcacgtaaccacatgctctgccactccatctttaggaacagctcctccaacaagcgctactgtaacgtcaacagtttctacagtcacgaccacgacttccacaccggctactactgcgtcctcgacttcaactacagtcacttcgaccgctacgaccaagacgaccacaacgacgtcgattgcaacgacaacccagccaagtagcagcagcgtatgtatcggtggaacgggccctgggaactatgttggtctttgcaatttctgttgccaatatggatattgccctcctggaccatgtacatgcacgcaatacggcgcccctgttccaactcctcctacgaatggaagggatggccgccctttgcctaatgaggatgactcttatttgggtttatgcagctttgcttgcaatcatggttactgccctgatacagcgtgtatgtacgtttag

SEQ ID NO: 8 - protein, 621 aa, Talaromyces bacillisporus
MMMKNVVFAMLSAFAGLVQGLPGSDRSLTARQSTDRLVFAHFMIGITSDRHSASDFDADMQRAKAYGIDAFALNIGVDPYTDEELGYAYDSAANNGMKVFISFDFNWWNTGQATAIGQKIAQYASKPAQLMVDNKVFVSSFAGDGVDVAGIRSAAGMDLFFAPNFHPSYGTDLSSVDGLLNWMAWPNNGNNKAPTAGANVTVQDGDEQYINALGGKAYIAPASPWFSTHFGPEVSYSKNWVFPSDLLWYNRWQDLLTLGPRFIEIVTWNDYGESHYIGPLDSPHTDDGASKWVNDMPHDGWLDMAKPFIAAFKDGATSIDSYITDDQLIYWYRPTPRGVDCDATDTCMVSAPNASGNYFIGRPNGWETMEDSVFVVSLFKSAATITVNSGGNVQTFNAPSGASSFEVPMGVGAQTFSVTRNGQTVFEGTSLKQIINGCVCGLYNFNAYVGSIPAGFADPLQPDGLASLTQGLHVTTCSATPSLGTAPPTSATVTSTVSTVTTTTSTPATTASSTSTTVTSTATTKTTTTTSIATTTQPSSSSVCIGGTGPGNYVGLCNFCCQYGYCPPGPCTCTQYGAPVPTPPTNGRDGRPLPNEDDSYLGLCSFACNHGYCPDTACMYV

SEQ ID NO: 9 - protein, 601 aa, Talaromyces bacillisporus
LPGSDRSLTARQSTDRLVFAHFMIGITSDRHSASDFDADMQRAKAYGIDAFALNIGVDPYTDEELGYAYDSAANNGMKVFISFDFNWWNTGQATAIGQKIAQYASKPAQLMVDNKVFVSSFAGDGVDVAGIRSAAGMDLFFAPNFHPSYGTDLSSVDGLLNWMAWPNNGNNKAPTAGANVTVQDGDEQYINALGGKAYIAPASPWFSTHFGPEVSYSKNWVFPSDLLWYNRWQDLLTLGPRFIEIVTWNDYGESHYIGPLDSPHTDDGASKWVNDMPHDGWLDMAKPFIAAFKDGATSIDSYITDDQLIYWYRPTPRGVDCDATDTCMVSAPNASGNYFIGRPNGWETMEDSVFVVSLFKSAATITVNSGGNVQTFNAPSGASSFEVPMGVGAQTFSVTRNGQTVFEGTSLKQIINGCVCGLYNFNAYVGSIPAGFADPLQPDGLASLTQGLHVTTCSATPSLGTAPPTSATVTSTVSTVTTTTSTPATTASSTSTTVTSTATTKTTTTTSIATTTQPSSSSVCIGGTGPGNYVGLCNFCCQYGYCPPGPCTCTQYGAPVPTPPTNGRDGRPLPNEDDSYLGLCSFACNHGYCPDTACMYV

SEQ ID NO: 10 - DNA, 2111 bp, Talaromyces apiculatus
  source 1..2111: mol_type = genomic DNA
atgatggtaaagagctttcttctcgcggcgttgtccgctgtgactaccctggttcagggacatccgggccacgaacgctccttgactgagagagcgtcaactgatagacttgtctttgctcatttcatggtatgtcaacctatattagaaagagtctgtcttaagaacaataatggctgacaagtgtagatcggcatcaccagtgaccgcaccagctcagccgactatgatgccgacatgcaacgagctaaatcgtacggcattgatgcctttgccctcaacattggtgtagacccctacaccgaccaacaactcggcttcgcctatgcgtcagccgcaaacaacgacatgaaagtcttcatctccttcgacttcaactggtggcacaccagccaagccaccgccatcgggcaaaagattgcccaatacgcaagtctacccgcccagctgaaggtcgatgacaaagtctttgtttcttcttttgccggagacggtgttgatatagctgctatgcgttcggcggctggcactgaactattttttgcgcctaatttccatccttcgtatggtacggatttgtcgaatgttgatggattgttgaattggatggcgtggccgaataatggaaataataaggcgccagatgctactggaaatgtgactgtggaacagggtgatcagcaatatgtttcggctcttgctggaaaagcttatattgctcgtaagtataactcgatgctatgacaaaggccagtgctgatatgaatagctgcctcgccctggttctcaactcattttggccccgaggtgtcctacagcaagaactgggtcttcccatctgatcttctatggtacaaccgctggcaagaactattgacgctaggtcctcgattcattgagattgtcacctggaacgactatggcgagtctcattatatcggacctcttgactctcctcacactgacgatggagcttccaaatgggtcaacgatatgtgagttccttccctcattagggagaaacatgattgctaatactttgtgaattaggccccacgacggctggctagacctcgcaaaaccattcatcgcagccttcaaagccggctccaccacagtagacagctacatcactagcgacgagctcatctactggtacaggcctacccccaaggacgtcgactgcgacgcaacagatacctgcatggatcccaatgccagcaactccagcggaaactacttcattggccgccctaatgggtaccagaccatggaagactccgtctttgtggtttcgttgttcactgccgccgctgagattactgtgacttctggcgggaattcacagaccttccaggcttcggctggtgcgaatgcgttccaggttcctatgggagtgggtacgcagacgttctccgtgactcggaacggggctactgtcttccagggaacgagtttaaagcagattattgatggatgtgtttgtgggttgtataactttaatgcttacggttcgtctccctgtttcccttgacatgcactaagctaaacccttttctttgggatggatagttggaacccttccagctggattcagtgatcctctcggacctgccggtctcgcatccctcgtccagggtcttcacgtctcaacctgcgctgcaactccaagtctaggaactgctcccccagtaaccacaacctcggtcccagtcactactaccaggagcactactaccacgactagcacaagcagcaccaaaaccaccagcacaaccacaacaacacccacaataacaactacatccaccactaagacgaccacatccacatccaccacatcatcaacaagcacctcaacatcaggcacagtctgtatcgccggcaccggaccaggaaactacgtcggtctctgcagtttctgctgcaattacggttattgtccaccggggccatgtacttgtaccgcctacggtgcgcctgtacctacgccaccggtgacgggtacggttggcttgccgttgtcgaacgaggatgattcgtatttggggttgtgtagttttgcttgtaatcatggttattgtccaagtacggcttgtagggttgcttag

SEQ ID NO: 11 - protein, 628 aa, Talaromyces apiculatus
MMVKSFLLAALSAVTTLVQGHPGHERSLTERASTDRLVFAHFMIGITSDRTSSADYDADMQRAKSYGIDAFALNIGVDPYTDQQLGFAYASAANNDMKVFISFDFNWWHTSQATAIGQKIAQYASLPAQLKVDDKVFVSSFAGDGVDIAAMRSAAGTELFFAPNFHPSYGTDLSNVDGLLNWMAWPNNGNNKAPDATGNVTVEQGDQQYVSALAGKAYIAPASPWFSTHFGPEVSYSKNWVFPSDLLWYNRWQELLTLGPRFIEIVTWNDYGESHYIGPLDSPHTDDGASKWVNDMPHDGWLDLAKPFIAAFKAGSTTVDSYITSDELIYWYRPTPKDVDCDATDTCMDPNASNSSGNYFIGRPNGYQTMEDSVFVVSLFTAAAEITVTSGGNSQTFQASAGANAFQVPMGVGTQTFSVTRNGATVFQGTSLKQIIDGCVCGLYNFNAYVGTLPAGFSDPLGPAGLASLVQGLHVSTCAATPSLGTAPPVTTTSVPVTTTRSTTTTTSTSSTKTTSTTTTTPTITTTSTTKTTTSTSTTSSTSTSTSGTVCIAGTGPGNYVGLCSFCCNYGYCPPGPCTCTAYGAPVPTPPVTGTVGLPLSNEDDSYLGLCSFACNHGYCPSTACRVA

SEQ ID NO: 12 - protein, 608 aa, Talaromyces apiculatus
HPGHERSLTERASTDRLVFAHFMIGITSDRTSSADYDADMQRAKSYGIDAFALNIGVDPYTDQQLGFAYASAANNDMKVFISFDFNWWHTSQATAIGQKIAQYASLPAQLKVDDKVFVSSFAGDGVDIAAMRSAAGTELFFAPNFHPSYGTDLSNVDGLLNWMAWPNNGNNKAPDATGNVTVEQGDQQYVSALAGKAYIAPASPWFSTHFGPEVSYSKNWVFPSDLLWYNRWQELLTLGPRFIEIVTWNDYGESHYIGPLDSPHTDDGASKWVNDMPHDGWLDLAKPFIAAFKAGSTTVDSYITSDELIYWYRPTPKDVDCDATDTCMDPNASNSSGNYFIGRPNGYQTMEDSVFVVSLFTAAAEITVTSGGNSQTFQASAGANAFQVPMGVGTQTFSVTRNGATVFQGTSLKQIIDGCVCGLYNFNAYVGTLPAGFSDPLGPAGLASLVQGLHVSTCAATPSLGTAPPVTTTSVPVTTTRSTTTTTSTSSTKTTSTTTTTPTITTTSTTKTTTSTSTTSSTSTSTSGTVCIAGTGPGNYVGLCSFCCNYGYCPPGPCTCTAYGAPVPTPPVTGTVGLPLSNEDDSYLGLCSFACNHGYCPSTACRVA

SEQ ID NO: 13 - DNA, 3352 bp, Streptomyces yanii
  source 1..3352: mol_type = genomic DNA
tcccggagcgcagatatacgccgccggcgtacccgtcggcggcctcgaccgcgctcacggcggcctgcgcgagcaccagggacagttcgcccggcactcggccgccgttcaccaccccgggcaccgccccgccttcatcctgcggactgtgccgggtagcgccgtaccaggtcacacagtcaggatgcgccccggcggccccgtagcgcacctggtggccccggtcacgtaatgaacgacatcttccaatatcatgcgaatcttttgcgtcgaaagaccgccacatcgtgcgcctgaagtcaagacaggccacgggacagtttgagacccatcgtttcgcacacctactttctggacgttcttcgcaaaagccctttgtaagagtttcaattttgagtgatcaaggcaatattttgcgaacggcatagacgtctttgcggtccgctcctagcctgtggggcgtccgaagagcccccacgagccgcaaggacgacgatcacgtgaccccaccaccgaaacgcctcctgctcagacgctctctgtccgcttccctctccctggccctcgccgcgttcggcaccgccgccgcggtcgtactgtccagcgccccgcccgcccgggccgcgggtgtccccgcgccttcccccctcgcggtccccggccgcggtgcgaccgttccgttcaaggaacaggaagccgagtacgcgaccacgaacggcacgctgatcggacccaaccggctgtacggcacgctgccctccgaggcctccggccggcaggccgtgacgctggacgccgtcggcgagtacgtggaattcaccctcaccgccccggcgaacgcgatgtccttccgttactcgctgccggacagccccgacggaaccggccgtgacgcctcgatcgacgtgcgtgtgggcgacggggccccgaagagtgtcccggtcacatcgaagtacggctggtactacggcggttacccgttcaacaacaacccgggcgacaccaacccgcaccacttctacgacgaggcccggaccatgttcgggtcgacgctccccatcggcacgaaggtgcggctccaggtctcctccaccgccgcgtcgccgacgttcaccatcgacctggccgacttcgagcaggtcggcgccccggtcggcaggccgtcgggggcactggacgtggtcagtgacttcggcgccgacccgacaggcgccaccgattccaccggcaggatccaggcggccgtcgacgccggccgggcccagggcaaggaggtgtacatcccgcagggaaccttccaggtccgcgatcacatcgtcgtcgacaaggtgacactgcgcggggccggtccctggtacagcgtgctgaccgggcgcgatccgtcgaaccgcagcaaggcggtcggggtctacggcaaatacgccgcggacggcggcagcagcaatgtcaccctcaaggacttcgccatcatcggcgacatccgtgaacgggtggacaacgaccaggtcaacgccatcggcggggccatgtccaattcggtcgtcgacaacatctggatgcagcacaccaagtgtggcgcctggatggacggaccgatggacaatttcaccgtcaagaacagccgcattctggaccagaccgccgacggcgtgaatttccattacggcgtcacgaactccaccgtcaccaacacattcgtccgcaacaccggtgacgacggcctggctatgtgggcggagaacgtaccgaacgtgaagaacaagttcacgttcaacaccgtgatcctgccgatcctggcgaacaacatcgtgacgtacggcggcaaggacatcaccatctccgacaacgtcatgtcggacacgatcaccaatggcggcggtcttcatgtcgccaatcgctatcccggtgtgaattcgggtcagggcacggcggtctccggcatcacgacagccgcccggaacactctgatccgtaccgggaacaacgacttcaactggcggttcggggtcggcgcggtctggttcagcggactcaacgaacccatcaacgccacgatcaacatcaccgacaccgagatactcgacagctcgtacgccgcgatccatctgatcgagggcgcgaccaacgggctgcatttcgacaacgtgaggatcgacggcgcgggtacctacgccttgcagatccaggcgccggggacggccacgttcaccgatgtcgtcgccacgcacatcgcccagtcgaacccgatccacaactgtgtcggcagcggcttccagatcacccggggcagcggaaactccggctggtactccgacccgccggcctgcaccggcacctggccggacccggtgtggaccaacggcggggtgccgggaggcggcaccgacccgaccgacccgcccaccgacccgcccaccgaccctccggccgatcccggcaacatcgcgctgggccgaccggtcaccgaatcggggcacgcggacgtctacggtgcggccaacgccgtggacggcaatgccaacagctactgggagagcaccaaccacgccttcccgcagaccatcaccgtggacctgggcgctcccaaggccgtcaaacgcgtggtcctgaaactgcccccggcgactgcatgggcgacccgcacgcagacgctgagcgtgtcgggcagcgccgacaactcctcgtacagctcgctcaaggggtcggccggctatgtcttcgacccggccgacggcaatacggcgacgatcactctgcccggcacgtcggcccgctatctgcggctgaccttcaccgcgaacacggggtggcccgcgggtcagctctcggaactggaggcgtacaccagctgaggaatgcgaccggtcgccggacgggttcgggcccgtccggcgaccggccggacgttccgggcgggccaaggcccgtcccggctcagcccgcctcgatccgactcgtcccggctcagcccagctcagctcacgtcaggaagtcgcgggcgatcccttccgccacccgctccagcagcgggcccgcctgtgccatgcacacggccgggtccgattccagctccgtgagggcgtacgcgcgccggatgccggccctgcccagggcctccggaggcagtgccaggcggccacagaccgccaccacctccagtcccgccgcgcgggcggccgccgcgacgcccgccggggccttgccgtgcagggtctgctcgtcgagggaaccctcgcccgtgatcaccagcgtggcgcgggccagtgccggcgcgaagccgagtacgtcgagcatgacctcgatgccggggcggaaccgggcgccgagcgctaccagcgccccgtagccgata

SEQ ID NO: 14 - protein, 783 aa, Streptomyces yanii
VTPPPKRLLLRRSLSASLSLALAAFGTAAAVVLSSAPPARAAGVPAPSPLAVPGRGATVPFKEQEAEYATTNGTLIGPNRLYGTLPSEASGRQAVTLDAVGEYVEFTLTAPANAMSFRYSLPDSPDGTGRDASIDVRVGDGAPKSVPVTSKYGWYYGGYPFNNNPGDTNPHHFYDEARTMFGSTLPIGTKVRLQVSSTAASPTFTIDLADFEQVGAPVGRPSGALDVVSDFGADPTGATDSTGRIQAAVDAGRAQGKEVYIPQGTFQVRDHIVVDKVTLRGAGPWYSVLTGRDPSNRSKAVGVYGKYAADGGSSNVTLKDFAIIGDIRERVDNDQVNAIGGAMSNSVVDNIWMQHTKCGAWMDGPMDNFTVKNSRILDQTADGVNFHYGVTNSTVTNTFVRNTGDDGLAMWAENVPNVKNKFTFNTVILPILANNIVTYGGKDITISDNVMSDTITNGGGLHVANRYPGVNSGQGTAVSGITTAARNTLIRTGNNDFNWRFGVGAVWFSGLNEPINATINITDTEILDSSYAAIHLIEGATNGLHFDNVRIDGAGTYALQIQAPGTATFTDVVATHIAQSNPIHNCVGSGFQITRGSGNSGWYSDPPACTGTWPDPVWTNGGVPGGGTDPTDPPTDPPTDPPADPGNIALGRPVTESGHADVYGAANAVDGNANSYWESTNHAFPQTITVDLGAPKAVKRVVLKLPPATAWATRTQTLSVSGSADNSSYSSLKGSAGYVFDPADGNTATITLPGTSARYLRLTFTANTGWPAGQLSELEAYTS

SEQ ID NO: 15 - protein, 742 aa, Streptomyces yanii
AGVPAPSPLAVPGRGATVPFKEQEAEYATTNGTLIGPNRLYGTLPSEASGRQAVTLDAVGEYVEFTLTAPANAMSFRYSLPDSPDGTGRDASIDVRVGDGAPKSVPVTSKYGWYYGGYPFNNNPGDTNPHHFYDEARTMFGSTLPIGTKVRLQVSSTAASPTFTIDLADFEQVGAPVGRPSGALDVVSDFGADPTGATDSTGRIQAAVDAGRAQGKEVYIPQGTFQVRDHIVVDKVTLRGAGPWYSVLTGRDPSNRSKAVGVYGKYAADGGSSNVTLKDFAIIGDIRERVDNDQVNAIGGAMSNSVVDNIWMQHTKCGAWMDGPMDNFTVKNSRILDQTADGVNFHYGVTNSTVTNTFVRNTGDDGLAMWAENVPNVKNKFTFNTVILPILANNIVTYGGKDITISDNVMSDTITNGGGLHVANRYPGVNSGQGTAVSGITTAARNTLIRTGNNDFNWRFGVGAVWFSGLNEPINATINITDTEILDSSYAAIHLIEGATNGLHFDNVRIDGAGTYALQIQAPGTATFTDVVATHIAQSNPIHNCVGSGFQITRGSGNSGWYSDPPACTGTWPDPVWTNGGVPGGGTDPTDPPTDPPTDPPADPGNIALGRPVTESGHADVYGAANAVDGNANSYWESTNHAFPQTITVDLGAPKAVKRVVLKLPPATAWATRTQTLSVSGSADNSSYSSLKGSAGYVFDPADGNTATITLPGTSARYLRLTFTANTGWPAGQLSELEAYTS

SEQ ID NO: 16 - DNA, 2391 bp, Streptomyces alni
  source 1..2391: mol_type = genomic DNA
atgttaagctttgccgtagccgccgccgctgcccttacagccgtcactctcgcaggggcgcctgcggcacatgcagcaggtgtcccggccccatctcctgtgggaatcgctggtcggggagccaatgtcccatttaaagaacttgaagcggaatatgcaaatactaatggatctttaattggaccagatcgttattatggacatttgccaagtgaagcttctgggagacaagcggtgaccctgaatagcgttggacaatatgtcgaatttactttaacagcaccggcaaatgccatgtctttaaggtattcacttcctgatacttctgatgggaaaggaagggatgcatcattggatgtgcaagtgaatggatctagcgtcaaaactcttcccgttacctctaaatattcatggtattatggcggctatccctttaataataatccaggagattcaaatcctcaccatttctatgatgaagcacgaacaatgtttcctgctacatatccaattggaacaaagattagagttcaagtaacgtcaacagctcaatctccaagctttacaatagatcttgctgattttgaacaagtcgccggagccattggacaaccgagcggaagcattaatgctgtaacagattatggtgttgatcctactggagccacagattcaacagcgaaaatgcaagctgcggttaatgcagggcaagcccaacataaagttgtgtatgtcccacaaggaacttataccctgtatgatcacgtaattgttgatggagttactgtacaaggtgccggaccttggtatacggtgtttggtggacgggatccagtacaaagaaatagggccgctggcttttatggaaaatatgtcgctggggggggatatggcggtgccattcgcccacaagaagcgggtggtccctcgcaaaacgtgactcttaaagattttgccattattggagatatacaggaacgtgttgatgatgatcaagtaaatggtattggtggggcgatgtcaaattctacaatcgatgatttatggatacaacatgttaaagttggagcatggatggatggaccaatggatcattttacgattaagaattctaggattctcgatactacggccgatggagttaattttcacagtggagtaacaaattctacagtaacaaatacgttcgtcagaaacgtgggcgatgatgggttagctagttggcctgagaaagcacccaatgtttcagattcttttgatcataataccgttgtactgcctattttagccaataatattgttacctatggcggaaaggatttcaacctttcagataatgtgatggcagatacaattactaatggaggagggttgcatatagcaaatcgatatcccggagttaatggtggcggaccgaatggagtcagcggaaccataacagctgcaagaaatacacttatacgcactggaaattcggattataattggcaatttggtgtcggggccatttggtttgatggacttcaaggaaaagttgatgcaaatattcaaatttccgatactgatatccttgattcaagctatgaagccattcaaagcattgaatcgggcgtgacaggagcaacgtttaagaatattaatattgatggtgcaggaacttatgcgattcaagcacaagccgccgcagacttaacgtttattaacgtcaaagccaagaatatagcacaatcagccacacccattcataattgtgtgggaactgcattaaatataattgatggtggtggaaattctggactgacggggcaaacatgtacgggaacatggccaacccctgtctggacaaatggcggcgttccgacgggtggcggtggatctacgacaacgccaccaacaactccgcctacaactccacctacaacaccgcctacaacaccacctaccacaccccctacaactccgccgacaacaccacctgcgaatcctaatttggctcaaggccatgctgcaagcgatacagggcatgcggacgtatatgtggcaggaaatactgtagatggtaacgtgaatacatattgggaatcgactaattctgcttttccacaaagcttaactgtagatcttggggctgctaaaactgtagggcgccttgtccttaaattgccaacaaattggggaacacggacagaaacactttcggttcttggttcaacggataattcgtcatattcaacgcttaaagcatcagctggctatacatttactttaggtacagatacagccacagtgacgtttacgcccacaacggctagatacttgcgtcttacatttacaggaaatactgggtggcctgcagggcaattgagtgaattggaagcgtatgctagctaa

SEQ ID NO: 17 - protein, 796 aa, Streptomyces alni
MLSFAVAAAAALTAVTLAGAPAAHAAGVPAPSPVGIAGRGANVPFKELEAEYANTNGSLIGPDRYYGHLPSEASGRQAVTLNSVGQYVEFTLTAPANAMSLRYSLPDTSDGKGRDASLDVQVNGSSVKTLPVTSKYSWYYGGYPFNNNPGDSNPHHFYDEARTMFPATYPIGTKIRVQVTSTAQSPSFTIDLADFEQVAGAIGQPSGSINAVTDYGVDPTGATDSTAKMQAAVNAGQAQHKVVYVPQGTYTLYDHVIVDGVTVQGAGPWYTVFGGRDPVQRNRAAGFYGKYVAGGGYGGAIRPQEAGGPSQNVTLKDFAIIGDIQERVDDDQVNGIGGAMSNSTIDDLWIQHVKVGAWMDGPMDHFTIKNSRILDTTADGVNFHSGVTNSTVTNTFVRNVGDDGLASWPEKAPNVSDSFDHNTVVLPILANNIVTYGGKDFNLSDNVMADTITNGGGLHIANRYPGVNGGGPNGVSGTITAARNTLIRTGNSDYNWQFGVGAIWFDGLQGKVDANIQISDTDILDSSYEAIQSIESGVTGATFKNINIDGAGTYAIQAQAAADLTFINVKAKNIAQSATPIHNCVGTALNIIDGGGNSGLTGQTCTGTWPTPVWTNGGVPTGGGGSTTTPPTTPPTTPPTTPPTTPPTTPPTTPPTTPPANPNLAQGHAASDTGHADVYVAGNTVDGNVNTYWESTNSAFPQSLTVDLGAAKTVGRLVLKLPTNWGTRTETLSVLGSTDNSSYSTLKASAGYTFTLGTDTATVTFTPTTARYLRLTFTGNTGWPAGQLSELEAYAS

SEQ ID NO: 18 - protein, 771 aa, Streptomyces alni
AGVPAPSPVGIAGRGANVPFKELEAEYANTNGSLIGPDRYYGHLPSEASGRQAVTLNSVGQYVEFTLTAPANAMSLRYSLPDTSDGKGRDASLDVQVNGSSVKTLPVTSKYSWYYGGYPFNNNPGDSNPHHFYDEARTMFPATYPIGTKIRVQVTSTAQSPSFTIDLADFEQVAGAIGQPSGSINAVTDYGVDPTGATDSTAKMQAAVNAGQAQHKVVYVPQGTYTLYDHVIVDGVTVQGAGPWYTVFGGRDPVQRNRAAGFYGKYVAGGGYGGAIRPQEAGGPSQNVTLKDFAIIGDIQERVDDDQVNGIGGAMSNSTIDDLWIQHVKVGAWMDGPMDHFTIKNSRILDTTADGVNFHSGVTNSTVTNTFVRNVGDDGLASWPEKAPNVSDSFDHNTVVLPILANNIVTYGGKDFNLSDNVMADTITNGGGLHIANRYPGVNGGGPNGVSGTITAARNTLIRTGNSDYNWQFGVGAIWFDGLQGKVDANIQISDTDILDSSYEAIQSIESGVTGATFKNINIDGAGTYAIQAQAAADLTFINVKAKNIAQSATPIHNCVGTALNIIDGGGNSGLTGQTCTGTWPTPVWTNGGVPTGGGGSTTTPPTTPPTTPPTTPPTTPPTTPPTTPPTTPPANPNLAQGHAASDTGHADVYVAGNTVDGNVNTYWESTNSAFPQSLTVDLGAAKTVGRLVLKLPTNWGTRTETLSVLGSTDNSSYSTLKASAGYTFTLGTDTATVTFTPTTARYLRLTFTGNTGWPAGQLSELEAYAS

SEQ ID NO: 19 - DNA, 2226 bp, Oribacterium sinus
  source 1..2226: mol_type = genomic DNA
atgaagtttcggaagcagctcacccttttgctgactatgacaggtttaggactcctgagccacgggacggcgttcgcagagacaagaacggaattgtgggccaaagagtcccagtcgtatggtgctaaattaccatatttgcgttatgaagccgaggaaggcgtaggccgtgacgccgtacttaaacattctacggcatatgacgaggttgaaatggaagcgtctaatcagtcatacgtccaacttacaaaagaagacagctctctccgtttcaccgtcaagaaggctgctaacgctatgaccttgaggttcacaatgccagaagacgccagcggtgaacttgaaatatcagtagaacgcaacggtgaattattaggtaagaagacagtcgaactcgataattcctcagcatggcaatacgtcaaagagaacgacgtattcgacgagaacatcgctgatagtcacagccggttcagattcgacgaacgtcacttcttactcgagaacgataataaagagcttatggagctcgaggccggcgatgtgctgacgatccgacgtacggacaagaaggccgacgagttaggcattgatttcattgaacttgaacaagtccctgaggccaaaggtgctccgtccaatagtatcagtattacagatgcaccatattcggcagttccgaatgatggccaagatgactcacaggctttcttagacgctttgaaggatgcagatgcagaaaataaaacgttgtacatccctgaagggacttttgacttcgatcagaagctcgtcctgtcagccaccgacatgcgcataacgggtgcgggcatttggtatactcgcttacacttcacgagcgaagaacaagctggtggcgggattgaattcttggattctagttctaacgtcgaaatggacaatctctatatggacagtgagcttaagtcgcgcttccaccaagaagcgaactataaaggcattgctggggtcctgggcgaaaacagcaaactccacgatctttggttagaacacttcgaatgtggtatttgggttggggattacgttgaggcagacaaaatgaagtacacgaagaatctcacggtaagtaattcacgcataagagataatttcgcagatggagtcaatttcgcccaaggtacgaggaacagcaaagtacaaaatagcgacattagagggaacggagacgatggcttggccactttcgcgtctaaagctatcgtcaagattaaagagaacgtaaacggcgttgaacaagtccggtacattcacaccgagagcaaaccggctgaaaataacgctttcctgaataatacagtggaattgacatggcgggcatcaggaatcgcacttcatggtggagccaatcaccacatagaggggaacttagttaaagatataactagtggtccagggcttcgggttagtacggtatttcctgggtataatttcgacgataatcagaatatctcaatcaaacggaacctgcttattcaaacaggcacagataacgacttttatgggaacgcgctcgcgtcgatccacttcgaaaagttatatggcgacatgaagaagatcacagtcgccgacaattgtttaataaattcaccgcatgggaagtacagcggtaatttctacataccagaggaaggaacgacggagataacattgcgcaacaatgaggaaaagtctatagacgttgaaccgatgcttgccgaagaggagaagaattttgcacctctttcagaagaggagaaactcgttgaggaacaaaagaaagccgaggaacaaaagaaagctgaagagttgaagaaagcagcagaacaagctgaacaagcgaagcacgaaggagagcaaccggggtacgatggagcgctcaacattgaactccatgaccaagaggagatccccgagacggccaactttcggttatattggttttcgggtgaaacggaagagaatggacgaacggtgcgctattgggtcaagaagcttgagggtatccaccttgatgaatcaatggaatactcattggaagatggaacaaaagtatttaatttcacacctgacgatattcccgaatacattccgatctcaggcacagcattcgttgaagactattattatgagggcgaagattggattcgatgggagtcccctgagggagtcgaatacgtcaagatccgctactggtccctgaagtag

SEQ ID NO: 20 - protein, 741 aa, Oribacterium sinus
MKFRKQLTLLLTMTGLGLLSHGTAFAETRTELWAKESQSYGAKLPYLRYEAEEGVGRDAVLKHSTAYDEVEMEASNQSYVQLTKEDSSLRFTVKKAANAMTLRFTMPEDASGELEISVERNGELLGKKTVELDNSSAWQYVKENDVFDENIADSHSRFRFDERHFLLENDNKELMELEAGDVLTIRRTDKKADELGIDFIELEQVPEAKGAPSNSISITDAPYSAVPNDGQDDSQAFLDALKDADAENKTLYIPEGTFDFDQKLVLSATDMRITGAGIWYTRLHFTSEEQAGGGIEFLDSSSNVEMDNLYMDSELKSRFHQEANYKGIAGVLGENSKLHDLWLEHFECGIWVGDYVEADKMKYTKNLTVSNSRIRDNFADGVNFAQGTRNSKVQNSDIRGNGDDGLATFASKAIVKIKENVNGVEQVRYIHTESKPAENNAFLNNTVELTWRASGIALHGGANHHIEGNLVKDITSGPGLRVSTVFPGYNFDDNQNISIKRNLLIQTGTDNDFYGNALASIHFEKLYGDMKKITVADNCLINSPHGKYSGNFYIPEEGTTEITLRNNEEKSIDVEPMLAEEEKNFAPLSEEEKLVEEQKKAEEQKKAEELKKAAEQAEQAKHEGEQPGYDGALNIELHDQEEIPETANFRLYWFSGETEENGRTVRYWVKKLEGIHLDESMEYSLEDGTKVFNFTPDDIPEYIPISGTAFVEDYYYEGEDWIRWESPEGVEYVKIRYWSLK

SEQ ID NO: 21 - protein, 715 aa, Oribacterium sinus
ETRTELWAKESQSYGAKLPYLRYEAEEGVGRDAVLKHSTAYDEVEMEASNQSYVQLTKEDSSLRFTVKKAANAMTLRFTMPEDASGELEISVERNGELLGKKTVELDNSSAWQYVKENDVFDENIADSHSRFRFDERHFLLENDNKELMELEAGDVLTIRRTDKKADELGIDFIELEQVPEAKGAPSNSISITDAPYSAVPNDGQDDSQAFLDALKDADAENKTLYIPEGTFDFDQKLVLSATDMRITGAGIWYTRLHFTSEEQAGGGIEFLDSSSNVEMDNLYMDSELKSRFHQEANYKGIAGVLGENSKLHDLWLEHFECGIWVGDYVEADKMKYTKNLTVSNSRIRDNFADGVNFAQGTRNSKVQNSDIRGNGDDGLATFASKAIVKIKENVNGVEQVRYIHTESKPAENNAFLNNTVELTWRASGIALHGGANHHIEGNLVKDITSGPGLRVSTVFPGYNFDDNQNISIKRNLLIQTGTDNDFYGNALASIHFEKLYGDMKKITVADNCLINSPHGKYSGNFYIPEEGTTEITLRNNEEKSIDVEPMLAEEEKNFAPLSEEEKLVEEQKKAEEQKKAEELKKAAEQAEQAKHEGEQPGYDGALNIELHDQEEIPETANFRLYWFSGETEENGRTVRYWVKKLEGIHLDESMEYSLEDGTKVFNFTPDDIPEYIPISGTAFVEDYYYEGEDWIRWESPEGVEYVKIRYWSLK

SEQ ID NO: 22 - DNA, 2310 bp, Streptomyces galbus
  source 1..2310: mol_type = genomic DNA
gtcattacgtccttagctatggccgcagctgcagcccttacagcgattggtcttgcatcagctccggctgcgcacgctgccacgggagtgccggctccatctccagtgggtattcctggccgcggagctgatgtgccctttaaagaaaccgaagcggaatatgcatccactaatgggacacttattggaccagataggctgtatgggcatcttccgtcagaagcttctgggcgacaagccgtaacattggatgctgttggagaatatgttgaatttacactgaccgcaccggctaatgctatgtcattgcgatattcattacccgattctgctgatggacgcggacgcaatgcaacgttggatgtccaagttgatggcaaaagcgtaaagacactgcctgttacatcaaaatatggatggttttatggtggatatccttttaataataacccaggggatagcaatccacatcacttttatgatgaagcgcgcacaatgtttgctacaacatatcccgcaggtacaaaacttcgcgttcaagtcacttccacagtacaaagtccttcatttacaattgatcttgctgattttgagcaagttgcagcacctattgcaaaaccagcaggcgcaatagatgcggtggcggattatggtgcagatccgactggagcaacagatacaactgcgagaattcaagctgcagtagatgcagggcatgctcaagggcgggctgtgtatgtcccgaaaggagattatacactgtatggtcatgtcattgttgatggggtaacggtacaaggagcgggaccgtggtatactgtatttggtggacgggaccctgtggatcgttctagggctgcaggattttatggaaaatacgttacaaatggtggaccatcacgcaatgtaacattaaaagactttgcaataattggagatattcaggaacgtaacgatgatgctcaggtaaatgcacttggtggagcaatgtcaaattccacagttgatcatctttggatccaacacgtaaaagttggggcttggatggatggccccatggatcattttacgattaagaattctcgtattttggatacgacggcagatggcgtcaattttcatacgggcgttacagattctactgttacaaatacatttgtacggaatacaggagatgatgggttagcatcgtggcctcaagataaaccaaatgttaatgatagttttgatcataatacggttgtccttccgatccttgcaaataatattgtgacatatggcggaaaagattttaacctctctgataatgtgatggctgatacaattacgaatggtggtgggcttcatattgcaaatagatatccaggagtgtccgcctcagctggaacagcagtggcaggaacaattactgctgcgcgtaatacacttattcggacgggaaattccgattataattggaattttggagttggggcgatttggtttgatgggctccaaggaaaagtagatgcagatattagaattagcgatacggatatacttgattcgtcttatgaagcaattcaaagcattgaatctggggttacaggggttacaatgaagaatgttaatattgatggagcaggaacatacgcaattcaagctcaagcggccgcaacaatgacttttataaatgtaacggctaagcatataggacaagcggcaactccaatacattcatgtgtaggatcagatctcagaattacggatggcggtggtaatagcggatggaatacagggcaaacatgtactggaacatggccggcacctatttggacaaatagtggggtccctagtggcggcggaacgacacccccgacgacgccgcctacaactccgccggcaccgagcgatgtaaatctggcgaaaggaagacctgtagtggatacagggcatgctgatgtgtatacagctgggaataccgtagatggaaatgccaatacttattgggaatcatctaataatgcttttccgcaatctttaaccgttgatcttgggtcagctcgtacagtcggaaggttggttcttaagctcccaccagccacagcgtggtcaacaagaacacaaacattgtctgtccttggtagcactgataattcaggttggacaacattgaaaagcagtgcgggatatacatttaatccatcttcaggtaatagtgttacattgaccctgaattcagctactacaaggtacttaagacttacatttacgggtaatacgggttggcctgccggacaacttagcgagcttgaagcatattcgagctaa

SEQ ID NO: 23 - protein, 769 aa, Streptomyces galbus
VITSLAMAAAAALTAIGLASAPAAHAATGVPAPSPVGIPGRGADVPFKETEAEYASTNGTLIGPDRLYGHLPSEASGRQAVTLDAVGEYVEFTLTAPANAMSLRYSLPDSADGRGRNATLDVQVDGKSVKTLPVTSKYGWFYGGYPFNNNPGDSNPHHFYDEARTMFATTYPAGTKLRVQVTSTVQSPSFTIDLADFEQVAAPIAKPAGAIDAVADYGADPTGATDTTARIQAAVDAGHAQGRAVYVPKGDYTLYGHVIVDGVTVQGAGPWYTVFGGRDPVDRSRAAGFYGKYVTNGGPSRNVTLKDFAIIGDIQERNDDAQVNALGGAMSNSTVDHLWIQHVKVGAWMDGPMDHFTIKNSRILDTTADGVNFHTGVTDSTVTNTFVRNTGDDGLASWPQDKPNVNDSFDHNTVVLPILANNIVTYGGKDFNLSDNVMADTITNGGGLHIANRYPGVSASAGTAVAGTITAARNTLIRTGNSDYNWNFGVGAIWFDGLQGKVDADIRISDTDILDSSYEAIQSIESGVTGVTMKNVNIDGAGTYAIQAQAAATMTFINVTAKHIGQAATPIHSCVGSDLRITDGGGNSGWNTGQTCTGTWPAPIWTNSGVPSGGGTTPPTTPPTTPPAPSDVNLAKGRPVVDTGHADVYTAGNTVDGNANTYWESSNNAFPQSLTVDLGSARTVGRLVLKLPPATAWSTRTQTLSVLGSTDNSGWTTLKSSAGYTFNPSSGNSVTLTLNSATTRYLRLTFTGNTGWPAGQLSELEAYSS

SEQ ID NO: 24 - protein, 743 aa, Streptomyces galbus
ATGVPAPSPVGIPGRGADVPFKETEAEYASTNGTLIGPDRLYGHLPSEASGRQAVTLDAVGEYVEFTLTAPANAMSLRYSLPDSADGRGRNATLDVQVDGKSVKTLPVTSKYGWFYGGYPFNNNPGDSNPHHFYDEARTMFATTYPAGTKLRVQVTSTVQSPSFTIDLADFEQVAAPIAKPAGAIDAVADYGADPTGATDTTARIQAAVDAGHAQGRAVYVPKGDYTLYGHVIVDGVTVQGAGPWYTVFGGRDPVDRSRAAGFYGKYVTNGGPSRNVTLKDFAIIGDIQERNDDAQVNALGGAMSNSTVDHLWIQHVKVGAWMDGPMDHFTIKNSRILDTTADGVNFHTGVTDSTVTNTFVRNTGDDGLASWPQDKPNVNDSFDHNTVVLPILANNIVTYGGKDFNLSDNVMADTITNGGGLHIANRYPGVSASAGTAVAGTITAARNTLIRTGNSDYNWNFGVGAIWFDGLQGKVDADIRISDTDILDSSYEAIQSIESGVTGVTMKNVNIDGAGTYAIQAQAAATMTFINVTAKHIGQAATPIHSCVGSDLRITDGGGNSGWNTGQTCTGTWPAPIWTNSGVPSGGGTTPPTTPPTTPPAPSDVNLAKGRPVVDTGHADVYTAGNTVDGNANTYWESSNNAFPQSLTVDLGSARTVGRLVLKLPPATAWSTRTQTLSVLGSTDNSGWTTLKSSAGYTFNPSSGNSVTLTLNSATTRYLRLTFTGNTGWPAGQLSELEAYSS

SEQ ID NO: 25 - DNA, 2184 bp, Streptomyces glomeratus
  source 1..2184: mol_type = genomic DNA
atgcatggaacaacgcggacagttaggagcgtacggagaatgccggccataggagccgctgtagctcttgctgcgggaatgctggtgactctgggagccccagcggcacatgcagatgctggagcgtctcttccatttacttcagtggaagcagaatcagcaacgacaaatggtaccaaaattggaccggattatacacaaggatctttagcgagcgaagcgtcagggagacaagcagtacgtttgacaagcggccaacgagtggaatttactgttccgagggcggcaaatgctgtaaatttggcatattctgtcccagatgggcaatcaggaacactgaatgtctatgttaatggtactcgcttgacaaagacgcttgctgttacgagtaaatattcatatgttgatacaggttggattccgggagcgcgcacgcatcatttctttgataatgcgcgtctcttattgggacaaaatgtgcaaccaggggatcgtattgcgtttgaaagcacaggcacacaagtaacagttgatgtggctgattttgaacaagtcgcggcggcggcatcacaaccggcagggtcagtgtcagttacgtctaaaggtgcagatccgagtgggcaaggggatagcacacaagcgtttcgagatgctattgcagcagctcaaggtggcgttgtttggattccacccggggattatcgtcttacatcaagtttatccggagttcaaaatgttacattgcaaggagctggatcttggtggtcggtagtacacacatctcgatttattgatcaatcgggatcatcggggaatgttcatattaaagattttgcagttttgggagaagttactgaaagagtcgattcatcaccggataattttgtaaatgggtccttaggaccaaatagcagcgttagcggtatgtggatacaacacttaaaagttgggttatggctcacagggaataatgataatcttgtagtggaaaataatcgtattcttgatacaacggcggatggactgaatctgaatggtactgctaaaggggttcgtgtacgtaataattttctgagaaatcaaggagacgatgcattagctatgtggtctttatatagcccagatacggattcttcattcgagaataataccatatcccagccgaatctggcaaatggtattgccatttatggtggtacagatttagcagtcagaaataatctcatttctgatacgaatgctctgggatccggcattgctattagcaatcagaaatttctggatcccttttctcctttggctggaacaattacggtagatgggaatacactggttcgtacgggagccatgaatccgaattggaatcatccgatgggagcattgcgggttgatagctatgactcggcaattaatgcaactgtaaatattacaaatacgactataacagattctccctattctgcgtttgaatttgtatcaggggggggacaaggataccctgtgagaaatgtgactgtcgatggggcaacggtccgaaatacaggaacagtagtggttcaagcagaagcaccaggtgcggcgacacttcggaacgtgacagctacgtcagttgggtcagcaggggtatataattgtccatatcctgcaaattcgggtgggtttgccttgacggatggtggtggaaatagcgggtggaattcaacatggtcagattgttctacatggccacaaccgggacaaggtaatccaccaccagatccaggacgaaatctggcaaaaggacgtcctgctacggcttcgggaagccaagatgtttatacacctggaaaagcagtagatggtgatgccaattcctattgggaatcaacaaataatgcgtttccgcaatcatttacggtggatttggggtccagccaagctgtcagacgtttagtcttgaagcttccacctcttagcgcgtggcaagcccgtgctcaaacgctctctgtcttagggtccacagatggttcgggttatacaacagtcgttggaagtgctgattatagatttgatcctgcaacaggaaatactgttacagtaacgcttccaggcggtactaatttaagataccttcgcttaaatgtaacagctaatactggatggcctgcagcccaattttcggaagttgaagcatatctttcttaa

SEQ ID NO: 26 - protein, 727 aa, Streptomyces glomeratus
MHGTTRTVRSVRRMPAIGAAVALAAGMLVTLGAPAAHADAGASLPFTSVEAESATTNGTKIGPDYTQGSLASEASGRQAVRLTSGQRVEFTVPRAANAVNLAYSVPDGQSGTLNVYVNGTRLTKTLAVTSKYSYVDTGWIPGARTHHFFDNARLLLGQNVQPGDRIAFESTGTQVTVDVADFEQVAAAASQPAGSVSVTSKGADPSGQGDSTQAFRDAIAAAQGGVVWIPPGDYRLTSSLSGVQNVTLQGAGSWWSVVHTSRFIDQSGSSGNVHIKDFAVLGEVTERVDSSPDNFVNGSLGPNSSVSGMWIQHLKVGLWLTGNNDNLVVENNRILDTTADGLNLNGTAKGVRVRNNFLRNQGDDALAMWSLYSPDTDSSFENNTISQPNLANGIAIYGGTDLAVRNNLISDTNALGSGIAISNQKFLDPFSPLAGTITVDGNTLVRTGAMNPNWNHPMGALRVDSYDSAINATVNITNTTITDSPYSAFEFVSGGGQGYPVRNVTVDGATVRNTGTVVVQAEAPGAATLRNVTATSVGSAGVYNCPYPANSGGFALTDGGGNSGWNSTWSDCSTWPQPGQGNPPPDPGRNLAKGRPATASGSQDVYTPGKAVDGDANSYWESTNNAFPQSFTVDLGSSQAVRRLVLKLPPLSAWQARAQTLSVLGSTDGSGYTTVVGSADYRFDPATGNTVTVTLPGGTNLRYLRLNVTANTGWPAAQFSEVEAYLS

SEQ ID NO: 27 - protein, 689 aa, Streptomyces glomeratus
DAGASLPFTSVEAESATTNGTKIGPDYTQGSLASEASGRQAVRLTSGQRVEFTVPRAANAVNLAYSVPDGQSGTLNVYVNGTRLTKTLAVTSKYSYVDTGWIPGARTHHFFDNARLLLGQNVQPGDRIAFESTGTQVTVDVADFEQVAAAASQPAGSVSVTSKGADPSGQGDSTQAFRDAIAAAQGGVVWIPPGDYRLTSSLSGVQNVTLQGAGSWWSVVHTSRFIDQSGSSGNVHIKDFAVLGEVTERVDSSPDNFVNGSLGPNSSVSGMWIQHLKVGLWLTGNNDNLVVENNRILDTTADGLNLNGTAKGVRVRNNFLRNQGDDALAMWSLYSPDTDSSFENNTISQPNLANGIAIYGGTDLAVRNNLISDTNALGSGIAISNQKFLDPFSPLAGTITVDGNTLVRTGAMNPNWNHPMGALRVDSYDSAINATVNITNTTITDSPYSAFEFVSGGGQGYPVRNVTVDGATVRNTGTVVVQAEAPGAATLRNVTATSVGSAGVYNCPYPANSGGFALTDGGGNSGWNSTWSDCSTWPQPGQGNPPPDPGRNLAKGRPATASGSQDVYTPGKAVDGDANSYWESTNNAFPQSFTVDLGSSQAVRRLVLKLPPLSAWQARAQTLSVLGSTDGSGYTTVVGSADYRFDPATGNTVTVTLPGGTNLRYLRLNVTANTGWPAAQFSEVEAYLS

SEQ ID NO: 28 - DNA, 2028 bp, Tetrasphaera sp-0185
  source 1..2028: mol_type = genomic DNA
atgaacgatcatccccactcacgtttggcacaactgaccagccggcgcccttcgcgcccggtgacggggatccttgcaatggcacttgcagcgactggagccatggttggtgttcttccgagtgctcaagcaggcgaacctaccgctcctgctacaacggctcccaaaataactcgaacagcactcgatccatcactggtgcaaggtagaggagcgacagttccttttgttgaacaagaagcagaaaatgcagcaacagatggacaagtattggccccaggacgtgaagcatatacgcttaatgctgaagctagtggccgtagcgccgtccgtctcaatcaacctggtcaatatgtggaatttactttaacgggcgatacgaatgcatttacattgagatatagtattccagatgctccagcaggcggcggaattacgtcaccgttaaccgttacagtagatggacatcatgattcaaccatgactttaacgagccaatattcctggttatatggcatgtatccattttcaaatgatcctaatgtggatccgaatccgggatggtggaaaccagaaccggatccagttactaaaccatttcgtccaaatcacttttatgatgaacaacgagtacttcttggtaaaacatatcatgccggcgataggatacgttttacggttccgcaaggcgcggcggccgcttggactgttcttgatgtagctgattttgaattggtcccgaaaccccttgtaaaaccacatggggctttgagcgttttagattttggtgcggatcctacgggcgcacgtgaaagtagcgacgcaattgatgctactattgcagcagccaaaacgcggggattaactgttttcataccgaaaggaacatatcaagtcaatagacatattgtagtagatgatgtcacagtgcaaggcgccggatcttggtggaccattattaaaggtcatctgattcctttagcgaccccggctcctgataaaagcacccatagcgcacctggattttatggaaaatacgctgcagatggcggaagtcataatgtgcatctgagcgattttgcaattgaaagtgatgtccgtgaacgaattgatacagatcaagtcaatggtattggcggcgctctgggcggcgggtctactgtggaacggttatacattcatcatacaaaagctggagtgtggcttgatggacctctttctggccttacacttaaagataatgtaattgcggatcaaatcgcggatggtattaatttacatcaaggagtaagtgacgttcgggctacaaataattttctccgaaatacaggtgatgatggaatggctatgtggtctgaaaatgtgaatgggcctcaagatgcagatcacgataatgtgtttgatcataatacagtgcaaacgcctgtcctcgcaaatgatatagcaatttatggtggtcatgataatacggtgtcagataatcttgtagccgatcccatacgggaaggatcgactcttcatgctggtgcacgttttaatgaagttccgttttccggaacaacaacatttacgcgcaatacaaccgtccgcggtgggcctagagatcttaattgggatcttggcctgggatcagtttggttttaccttttagataattcaatgcatggtcaaatctatgtaaccgactcttcaattctggactccacgtataatgcattcatgtttgtcgccgattggcccgtcaaagatacctacgatataacaaatgtacatatttctaatataaaagttgatggtacagggacaaacgtagtatcagccagggctgctggatgggcaacgtttgataacgtagtcgcccggaatgttggcgatccttttgtaaatgattgtggcacctttcattttactgggacccctgaatttgatgttcgcctgggagctggaaattcaggctggacacaatggggatcatggtgtcaagatcgccctacggtagtcccaccacctcctccgtcagcttggtaa

SEQ ID NO: 29 - protein, 675 aa, Tetrasphaera sp-0185
MNDHPHSRLAQLTSRRPSRPVTGILAMALAATGAMVGVLPSAQAGEPTAPATTAPKITRTALDPSLVQGRGATVPFVEQEAENAATDGQVLAPGREAYTLNAEASGRSAVRLNQPGQYVEFTLTGDTNAFTLRYSIPDAPAGGGITSPLTVTVDGHHDSTMTLTSQYSWLYGMYPFSNDPNVDPNPGWWKPEPDPVTKPFRPNHFYDEQRVLLGKTYHAGDRIRFTVPQGAAAAWTVLDVADFELVPKPLVKPHGALSVLDFGADPTGARESSDAIDATIAAAKTRGLTVFIPKGTYQVNRHIVVDDVTVQGAGSWWTIIKGHLIPLATPAPDKSTHSAPGFYGKYAADGGSHNVHLSDFAIESDVRERIDTDQVNGIGGALGGGSTVERLYIHHTKAGVWLDGPLSGLTLKDNVIADQIADGINLHQGVSDVRATNNFLRNTGDDGMAMWSENVNGPQDADHDNVFDHNTVQTPVLANDIAIYGGHDNTVSDNLVADPIREGSTLHAGARFNEVPFSGTTTFTRNTTVRGGPRDLNWDLGLGSVWFYLLDNSMHGQIYVTDSSILDSTYNAFMFVADWPVKDTYDITNVHISNIKVDGTGTNVVSARAAGWATFDNVVARNVGDPFVNDCGTFHFTGTPEFDVRLGAGNSGWTQWGSWCQDRPTVVPPPPPSAW

SEQ ID NO: 30 - protein, 631 aa, Tetrasphaera sp-0185
GEPTAPATTAPKITRTALDPSLVQGRGATVPFVEQEAENAATDGQVLAPGREAYTLNAEASGRSAVRLNQPGQYVEFTLTGDTNAFTLRYSIPDAPAGGGITSPLTVTVDGHHDSTMTLTSQYSWLYGMYPFSNDPNVDPNPGWWKPEPDPVTKPFRPNHFYDEQRVLLGKTYHAGDRIRFTVPQGAAAAWTVLDVADFELVPKPLVKPHGALSVLDFGADPTGARESSDAIDATIAAAKTRGLTVFIPKGTYQVNRHIVVDDVTVQGAGSWWTIIKGHLIPLATPAPDKSTHSAPGFYGKYAADGGSHNVHLSDFAIESDVRERIDTDQVNGIGGALGGGSTVERLYIHHTKAGVWLDGPLSGLTLKDNVIADQIADGINLHQGVSDVRATNNFLRNTGDDGMAMWSENVNGPQDADHDNVFDHNTVQTPVLANDIAIYGGHDNTVSDNLVADPIREGSTLHAGARFNEVPFSGTTTFTRNTTVRGGPRDLNWDLGLGSVWFYLLDNSMHGQIYVTDSSILDSTYNAFMFVADWPVKDTYDITNVHISNIKVDGTGTNVVSARAAGWATFDNVVARNVGDPFVNDCGTFHFTGTPEFDVRLGAGNSGWTQWGSWCQDRPTVVPPPPPSAW

SEQ ID NO: 31 - DNA, 2436 bp, Kitasatospora phosalacinea
  source 1..2436: mol_type = genomic DNA
atgcaaaacccgccaactagagcgcgcagaactgtagcgtccctcacaactctggccttaggagcgacgggacttctcgcagttgcttttgtcaatgctggggcagcacatgcggcggccgtacctacattatcaccactcgcagttcccaatcggggagcaagtgttccctttgtagaacaagaagcggaatatgcagcaacgaatgggacggtcgttggaccggatcgacaatatggacatctcgctagcgaagctagtggtagacaagcggtcactttaaatgccgttgggcaatatgtagaatttaccttaacagcacctacaaatagtattgattttcgttattctctccctgattcggccgatgggaaaggaagggatgcaagtatggatcttttggttaatggtcagaagttaaagtcggtggatgttacgtctaaatattcttggtattatggtggctatccttttaataataatcctggagattcgaatcctcatcacttctttgacgaaactcgtactttgctcggttcaacatatcctgcaggcacaaaagtgcgtttgcaagtgtcatcgactgcccaatcacctacatttacagttgatttagcagattttgagaaagtggcagatcctattgcaaaaccgaccggtgcgattgatgtcgtggctgattatggggcggatccaacaggagcgactgattcaacagctaaatttcaagcagcagtcaatgcgggcgcagcacaaggaaggaccgtatatattcctcaaggtgcgtttactctttatgatcatgttgtagtagataaagtcactctcgcaggcgcaggaccgtggtattcagtattaggcggtagggatccaataaatagaaatcgtgccgcaggaatttatggaaaatatgtagcgggtggtggatatggtggtggaattcgacctcaagaagcaggcgggccaagtcaaaatgtaaccttaaaagattttgcaattattggtgatgttagagaacgggttgatgatgatcaagtaaatggattaggtggagctatgagcaattcaacagtggataatttgtggattcaacatacaaaagtaggagcgtggatggatgggccaatggatcattttacaattaagaatagtcggattctggatcaaacggctgatggagtgaattttcatacaggtgttacgaattctacagttacaaatacgtttcttcgaaatacaggggatgatggactcgcaagttggcccgaacgattagcgaatgtaaatgattcatttacacataataccgttgtcttacctattcttgcaaataatattgtaacctatggcggaaaggatttcacaatttcagacaatgttatggctgatactattactaatggtggcgggcttcatattgcaaatcgctatcctggggttacatcggggaatggaacagctgtgtctgggacaattactgctgcgcggaatacattgattcgggctggaaattcagattataattggcaatttggggtaggagcaatttggtttgatggtttaaatgagcctattacgggtgcaacaattaatatttccgatacagatattcttgattctagctatgaagcaattcagacaattgaaggcggggtgactggagttaatttgacaaatgtcaacattgatggtgctggaacgtatgctattcaagctcaagcaaatgcccaaatgaaatttacgaatgtgaaagcaaagaatattgctcaagcggcaacacctattcataattgtgttggaacaggatttcaaattacagatggtggtggaaattctgggctttcgggaagcacgtgtactggtcaatggccggcccctgtttggacatataatggtgtacctgtgggaggagggaatggtggaactagcagccctagcccatcacccagcccctctacgtccgtgccagcttccccaagtcccagcgcgccacctagcagtacatgtccatcagggactggtaatctggcgcaaggaagaccagcaacggctagcgggactagtcaaacgtatggcccggggaatgcagtagatggcgatgcaaatacttattgggaatcggcgaataatgcatttccacaatggattcaagtggatcttggatgtgcccggaatttgacaaaagcggtgttaaaattaccgccagcgacagcgtgggcaacaagaactcaaacagtgacggttcaaggaagcacagatggaacaacatttacaacgcttgttggagcgacaggatatacatttgatcctgcgacgggtaatacagttacggttaatttgccagctaattccacaaggtatttacgtttaacggtgacaggaaatacagggtggccggcgggccaactgtctgagctgcaagcctttactagctaa

SEQ ID NO: 32 - protein, 811 aa, Kitasatospora phosalacinea
MQNPPTRARRTVASLTTLALGATGLLAVAFVNAGAAHAAAVPTLSPLAVPNRGASVPFVEQEAEYAATNGTVVGPDRQYGHLASEASGRQAVTLNAVGQYVEFTLTAPTNSIDFRYSLPDSADGKGRDASMDLLVNGQKLKSVDVTSKYSWYYGGYPFNNNPGDSNPHHFFDETRTLLGSTYPAGTKVRLQVSSTAQSPTFTVDLADFEKVADPIAKPTGAIDVVADYGADPTGATDSTAKFQAAVNAGAAQGRTVYIPQGAFTLYDHVVVDKVTLAGAGPWYSVLGGRDPINRNRAAGIYGKYVAGGGYGGGIRPQEAGGPSQNVTLKDFAIIGDVRERVDDDQVNGLGGAMSNSTVDNLWIQHTKVGAWMDGPMDHFTIKNSRILDQTADGVNFHTGVTNSTVTNTFLRNTGDDGLASWPERLANVNDSFTHNTVVLPILANNIVTYGGKDFTISDNVMADTITNGGGLHIANRYPGVTSGNGTAVSGTITAARNTLIRAGNSDYNWQFGVGAIWFDGLNEPITGATINISDTDILDSSYEAIQTIEGGVTGVNLTNVNIDGAGTYAIQAQANAQMKFTNVKAKNIAQAATPIHNCVGTGFQITDGGGNSGLSGSTCTGQWPAPVWTYNGVPVGGGNGGTSSPSPSPSPSTSVPASPSPSAPPSSTCPSGTGNLAQGRPATASGTSQTYGPGNAVDGDANTYWESANNAFPQWIQVDLGCARNLTKAVLKLPPATAWATRTQTVTVQGSTDGTTFTTLVGATGYTFDPATGNTVTVNLPANSTRYLRLTVTGNTGWPAGQLSELQAFTS

SEQ ID NO: 33 - protein, 773 aa, Kitasatospora phosalacinea
AAVPTLSPLAVPNRGASVPFVEQEAEYAATNGTVVGPDRQYGHLASEASGRQAVTLNAVGQYVEFTLTAPTNSIDFRYSLPDSADGKGRDASMDLLVNGQKLKSVDVTSKYSWYYGGYPFNNNPGDSNPHHFFDETRTLLGSTYPAGTKVRLQVSSTAQSPTFTVDLADFEKVADPIAKPTGAIDVVADYGADPTGATDSTAKFQAAVNAGAAQGRTVYIPQGAFTLYDHVVVDKVTLAGAGPWYSVLGGRDPINRNRAAGIYGKYVAGGGYGGGIRPQEAGGPSQNVTLKDFAIIGDVRERVDDDQVNGLGGAMSNSTVDNLWIQHTKVGAWMDGPMDHFTIKNSRILDQTADGVNFHTGVTNSTVTNTFLRNTGDDGLASWPERLANVNDSFTHNTVVLPILANNIVTYGGKDFTISDNVMADTITNGGGLHIANRYPGVTSGNGTAVSGTITAARNTLIRAGNSDYNWQFGVGAIWFDGLNEPITGATINISDTDILDSSYEAIQTIEGGVTGVNLTNVNIDGAGTYAIQAQANAQMKFTNVKAKNIAQAATPIHNCVGTGFQITDGGGNSGLSGSTCTGQWPAPVWTYNGVPVGGGNGGTSSPSPSPSPSTSVPASPSPSAPPSSTCPSGTGNLAQGRPATASGTSQTYGPGNAVDGDANTYWESANNAFPQWIQVDLGCARNLTKAVLKLPPATAWATRTQTVTVQGSTDGTTFTTLVGATGYTFDPATGNTVTVNLPANSTRYLRLTVTGNTGWPAGQLSELQAFTS

SEQ ID NO: 34 - protein, 27 aa, Bacillus clausii
MKKPLGKIVASTALLISVAFSSSIASA

SEQ ID NO: 35 - protein, 8 aa, synthetic construct
  REGION 1..8: note = His-tag
HHHHHHPR

SEQ ID NO: 36 - protein, 610 aa, Trichoderma harzianum
APANVAIRSLEERASSADRLVFCHFMIGIVGDRGSSADYDDDMQRAKAAGIDAFALNIGVDGYTDQQLGYAYDSADRNGMKVFISFDFNWWSPGNAVGVGQKIAQYASRPAQLYVDNRPFASSFAGDGLDVNALRSAAGSNVYFVPNFHPGQSSPSNIDGALNWMAWDNDGNNKAPKPGQTVTVADGDNAYKNWLGGKPYLAPVSPWFFTHFGPEVSYSKNWVFPGGPLIYNRWQQVLQQGFPMVEIVTWNDYGESHYVGPLKSKHFDDGNSKWVNDMPHDGFLDLSKPFIAAYKNRDTDISKYVQNEQLVYWYRRNLKALDCDATDTTSNRPANNGSGNYFMGRPDGWQTMDDTVYVAALLKTAGSVTVTSGGTTQTFQANAGANLFQIPASIGQQKFALTRNGQTVFSGTSLMDITNVCSCGIYNFNPYVGTIPAGFDDPLQADGLFSLTIGLHVTTCQAKPSLGTNPPVTSGPVSSLPASSTTRASSPPVSSTRVSSPPVSSPPVSRTSSPPPPPASSTPPSGQVCVAGTVADGESGNYIGLCQFSCNYGYCPPGPCKCTAFGAPISPPASNGRNGCPLPGEGDGYLGLCSFSCNHNYCPPTACQYC